SEQUENCE LISTING (28 sequences)

SEQ ID NO: 1 - DNA, 1569 bp, Erwinia amylovora
  source 1..1569: mol_type = unassigned DNA
atgagccaacaagtcattatttttgataccactctgcgcgatggcgagcaggcattacaggccagtctgagtgtgaaagaaaagctgcagattgccctggcgctggagcggatgggggttgatgtcatggaggtcggtttcccggtctcttcccccggggattttgaatcggtacaaaccattgcccgccagatcaagagcagccgggtgtgtggcctggcacgctgtgtggaaaaggatatcgacgcggcttatgaagcgctgcgcgtggcggatgcctatcgtattcacaccttcctggcgacctcaccgatgcatatcgccaccaaactgcgcagcaccctgccggaagtgattgaacgcgccgtgaagatgattaagcgcgcacgcaattacaccgacgacgtggagttctcctgcgaagacggcggtcgtaccccgattgacgacctgtgccgcgtggtggaagccgcgattaacgccggtgccaccaccatcaatatccccgataccgtgggttataccttaccttacgaatacgccagtattttcagcgcgctgcgcgcacgcgtgccgaatattgataaagcgatcctgtcggtgcatacccacgacgatctgggcatggccgtcggtaatgcgctggcggcggtgaatgccggtgcgcgtcagatcgaaggggcaatgaacggactgggtgaacgcgccggtaactgcgcgctggaagagaccattatggcgatcaaaactcgtggtcagctgatgaacgtgcatacgcgcatccatcaccaggaaatctaccgcacctgtcagaccgtgagcaaaatctgcaatatgccgatcccggcgcataaggcaattattggatccaacgcatttgcgcactcttccggtattcaccaggacggcgtactgaaaaaccgcgaaaactacgagatcctgacgccggaatcgatcggcctgaaacaggtacagttgaacctgacctcgcgttccggccgcgccgccgtgaaacaccgcatggaagagatgggctacggcgaagacgaatacaacatggacaggctgtacgacgcgttcaaggcgctggccgataaaaaaggccaggtgtttgattacgatctggaggcgctggcatttatcagtcaacagtcggaagaaccggaatacttcaggctggatacgttcaacgtgcagtccggctccagcgtcattgccaccgccaccgtgcagctgtgctgcggcgaccaacagaaaaccgaagccgctaccggaaacggtccggtcgacgcggtttatcaggcaataaaccgtatgactgaattcaacgccgagctgatcagttatcagttgaccgccaaaggccacggtaaagacgcgctgggccaggtcgatattgtggtgcagtacaacggccgcaaattccacggcgtcggcctggcgaccgatatcgtcgaatcctcggcaaaagcgatggtaaacgcgctgaacaccatctggcgcgcgcgtcaggttgagcaggaattacagcgtaagtcccaggtcaaagatcacaaggaaaccgtataa

SEQ ID NO: 2 - DNA, 1089 bp, Erwinia amylovora
  source 1..1089: mol_type = unassigned DNA
atgtcccgtacttaccatattgcagtgttacccggtgacggcatcggcccggaagtgatggctcaggccagcaaggtgctcgacgctatccgccagcgctttgatctgcgtatcactaccagcgaatacgacgtcggcggtattgctatcgaccgtcacggcgtgccgctgccggacagcacggtaaaaggctgtgagcaggccgatgccatcctgtttggctcggtcggcgggccaaagtgggaacatctgcccccggcagaacagcccgaacgcggcgcgctgctgccgctgcgtaaacactttaagctgttcagcaacctgcgtccggccagcctgtaccaggggctggaggatttttgcccgctgcgtcgggatattgccgatcgcggcttcgatattctggtggtgcgcgagctgaccggcgggatctactttggtcagccgaagggccgcgaaggcagcgggatgcatgaacgcgcctttgataccgaagtctattaccgctttgaaatcgagcgcattgcacgtattgcctttgaatcagcgcgtaaacgccgtcacaaagtgacctcgattgacaaagccaacgtgctgcaatcctccattctctggcgtgaaatcgtgagtgaggtggcacaggattacccggatgttgcgttaagccatatgtatatcgacaacgccaccatgcagctaatcaaagcgccatcgcagttcgacgtgatgctgtgctcgaatctgttcggcgatatcctgtcggatgaatgcgcgatgatcaccggttcaatgggcatgctgccttccgccagccttaacgagcagggctttggcctgttcgaaccggcaggcggctccgcaccggatatcgccggtaaaaacatcgccaacccggtggcgcaaattctgtcgctggcgctgctgctgcgctacagtctggatgcagccgcagcggccgacgccattgagcgggcgatcaaccgggcgctggaagagggtcaccgtaccggagacctggccggaaacggcaaggccatcggcaccgatgaaatgggcagcgtgattgcccgatttattcgcgaagcataa

SEQ ID NO: 3 - DNA, 1398 bp, Erwinia amylovora
  source 1..1398: mol_type = unassigned DNA
atgaagacactgtacgagaagctgtttgatgcgcatgtcgtgcatgaaacccacggcgaaacgccgctgatttatatcgacaggcacctggtgcatgaagtgacctctgcccaggcctttgacggtctgcgcgcccacggtcgtgcggtgcgccagccgtcgaaaacctttgccaccatggatcacaacgtgtctacccagtcgcgcgatattaacgccagcggcgaaatggcgcgcatccagatgtcaacgcttgtgaaaaactgtgaagcctttggcgtacagctgtttgacctcagtcacccgttccaggggatcgtccatgtgatggggccggaacaggggctgtcgctgcccggcatgaccctcgtctgcggtgattcccacacatccacccacggcgctctgggggcgctggcgttcggcatcggcacctcggaagtggaacacgttctggctacccagacactgaagcagtcacgcgccaaaaccatgaagatcgaagtgcgtggtgacacggcggcgggcatcaccgccaaagatattgcgctggcgattatcggcaaaaccggcagcggcggtggtaacggtcacgttgtcgaattctgcggtgcggcggtggaagccctgagcatggaaggccgcatgacgctgtgtaatatggcgattgagctgggcgctaaggccggattaatcgccccggacgacaccacgttcagctacgtaaaaggccgtcgttatgcccccaaagaggccgaatggcagcaggcggtcgactactggcgcaccctgcacaccgatgagggcgcgcgctttgacaaggtggtgattatcgatgctgcggccattgccccccaggtaacctggggaacccatcccggacaggtgatcgccgtcagcgatccgatccccgatccggcctctttcagcgatccgctggagcgcgcctcggcggaaaaagcgctggcctatatggggctcaagcccggtatccgtctgaccgacgtccccatcgataaggtgtttatcggttcctgtaccaactcgcgcattgaagatctgcgtgcggcggcagcaatcgtcaaagggcaaaaaattgccaagggcgtacagggttatgtggtgccaggttccggcccggtgaaggcgcaggcggaagcggaagggctggatcgcatctttattgctgccggatttgaatggcgtttgccgggctgttcaatgtgcctggcgatgaataatgaccgcctgcagccgggtgaacgctgtgcgtcaaccagcaaccgtaactttgaaggccgccaggggcgcggcgggcttactcatctggtcagtccggcgatggcggcagcggcagcggtagccggtcactttgccgatatccgcgagctggcgtaa

SEQ ID NO: 4 - DNA, 930 bp, Erwinia amylovora
  source 1..930: mol_type = unassigned DNA
atgagtcagaaatatcgcgtgtttatcgacggccaggcaggcacgaccggattgcagatccagaaacgattagcccgtcaccctcagattgaaatgctacagattgatgaagcacagcgcaaagatgccgccgcccgtcaggcgctgatgaaacaggctgatgtcagtatcctttgcctgccggatgccgcggcgcgggaagcggtagcgctggccgacgaggtgggtgcacgcgtgctggatgccagttccgctcaccgggtggatgccggctgggtatacggcctgccagagcttgctcctgaacaacgtgataaaatcagcggcgccagccatgtatctaacccgggctgttatgctacgggggctattgccctgctggcgccactgattcgaaatggtctgttgaagccagactgccagctggcaatcaacgcgatttccggttacagcggcggcggcaagcagatgatcgagcggtattgccaggcgggcaccggttatgctgcttacggtctggactttgatcataaacatattaaagagattaaggcgtggagcggtctgagtagccggcccattttacaacctgcggtcggtgactacgcacagggcatgctggtgttcatccctttaaatggcattgacagcgagccgttacagcaggcgctggcagactattacgccggccaactgtttattaaggtcaatcccctgaacgccatggatgagcacagcgcgccctatctgctgccggaagcgctgaataataccaatcaattagagctgttcgtcttcagccacccacagcagcgtcagtgcattttagcatcacggctggataacctgggtaaaggggcttcgggtgccgccgtgcagaacctgaacctgatgctcggtctggcagaagagacctgcgttaacctctga

SEQ ID NO: 5 - DNA, 1218 bp, Erwinia amylovora
  source 1..1218: mol_type = unassigned DNA
atggcggcggaaaaaatagcggtaacgcgggcgacatttgacgaggtaattttgcctgtttatgcaccagcgcagtttgtgccggtcaaaggcaaaggcagccgtatctgggaccagcagggtaaagagtatatcgatttctccggtgggattgcggttaccgcgctgggccactgccatccggcgctggtggaagcgttaaaaacccagggcgaaaccctgtggcacaccagtaacgttttcaccaatgagccagccttgcgcctggcgcgtaaactgatcgatgccactttcgccgagcgcgtattctttgccaactccggcgcggaagccaacgaagcggctttcaagctggcgcgttactacgcttcacagcgccacagcccgtataaaagcaaaattatcgctttccataatgcgtttcatggccgcacgctgtttaccgtttccgtcggcgggcagccaaaatactccgatggctttgggccaaaaccggccgatattgtccatgtgccctttaacgatctggcggcggtcaaagcggtcatcgacgatcatacctgtgcgattgtcgtcgagccgatccagggggagggcggcgtggtaccggccactgaggaattcatgcaggggctgcgtcagctgtgtgatgaacatcatgcgctgctggtgatggacgaagtacagagcggcatgggccgtagcggcaagctgtttgcctacgagcactacggcgtgcagccagatattgtcaccagcgcgaaagcactcggcggcggcttcccggtcagcgcgatgctgaccaccaacgctattgcctcagtgatggccccgggcgtgcacggcaccacctacggcggcaatccgttggcctgtgcggtggcggaagcggcgctggacattatcaatacgccggaagtgcttgacggcgtggccgcgcggcggcagctgtttgttgatgcgctacagtcgctgaataccaggcgggatctgttcagcgatatccgtggtaaagggctgctgattggtgcagcgctgaagccacagtacgctgataaagcccgcgatatccttaacgcggcggcggccgaaggggtgatggtgctggtcgccggcagcggcgtgatgcgcttcgcgccatcgctgttgattgagcctgtcgatattgccgaaggtatggagcgctttgctgcggccgtcgagaaggtgctggcgtag

SEQ ID NO: 6 - DNA, 1152 bp, Erwinia amylovora
  source 1..1152: mol_type = unassigned DNA
gtgaagacaaaattacccaattttattgagatttaccgccagttgattgcaacgccatcaatcagtgctaccgacagcgcgctcgatcaaagcaatgaaactttaatcaatttgctcggcggctggttccgcgatcttggcttcagcgttgaggtacagccggttcccggcacgcgcaataaattcaatatgctggccaggagcggacagggtgccggcggcctgctgctggcgggccataccgacaccgttccctttgacgatggccgctggacgcgcgatcccttcacgcttaacgagcatgacaataagctgtacggactcggcaccgccgatatgaaaggtttcttcgcctttattttagacacgctgcgtgatgttgacctgtctgcgctgaaaaaaccgctgtacgttctggccaccgccgacgaagaaacgacgatggccggtgcgaaattcttctctgaatccaccgctctgcgcccggactgcgccatcattggcgaaccaacctcgctaaaaccggtgcgcgcgcataaaggccatatctcaaacgtcatccgtattcaggggcagtccggccactccagcgatccgggccgcggcgtcaacgccattgaactgatgcacgaatccatcacccggttaacggggctgcgcaacacgctgaaagagcgctaccatcacgcgggttttgccattccttacccaacgatgaattttggccacattcacggcggggatgcggccaaccgtatctgcgcctgctgcgaactgcatatggatattcggccgctgccgggcctgacgctaagcgatctcgacgggctgctgaatgaggcgctggctccggtcagcgcgcgctggccgggccgtctgaccgttggcgagctgcacccgcccattccgggctacgagtgtccgcgtgaccaccagctggtgcaggtggttgaaaaactgctgggttgcgaaaccgaggtggtgaactattgcaccgaggcgccgtttattcagcaaatctgcccaacgctggtgttaggcccgggatcgatcgaccaggcgcatcagccggatgaatttatcgataccgcctttatcaagccaacgcgggaattaattgctcaggtggtgcaccacttttgccatcattag

SEQ ID NO: 7 - DNA, 1215 bp, Erwinia amylovora
  source 1..1215: mol_type = unassigned DNA
atgcaagacaaaagcatcaagaaaatcgtgctggcttactccggcggtctggatacttcagccatcattccgtggctgaaagagaactatggcggctgcgaagtggtggcgttcgtggcggatatcggccaggagcgtggcgacctggaaggcgtggagcagaaagcgctgcagtccggtgcgtcagagtgccatgtggtggatctgcgtgaagagttcatcagcgagtacgtttatccggtgctgcaaaccggtgccctgtatgaaggcacctacctgctgggtacgtcgatggcgcgcccgattatcgccaaagcccaggtagagctggcgctgaaagtgggagccgatgccctgtgccacggtgcaaccggcaaagggaatgaccaggtgcgtttcgaaaccacttacaccgcgctggcgccacagctgaaagtggtggcgccatggcgtgaatgggatctgcgctcgcgtgaggcgctgctgggctatctgaaagagcgtcatatcccgactaccgcatcgctggaaaaaatctacagccgtgacgaaaacgcctggcatatctccaccgaaggcggcgtgctggaaagcccgtggaacgccccgaacaaagactgctgggtgtggaccgtcgatccgctggaagcgccggaccagccagaacaggtcaccgttgccgttgagaaaggtcgcgtcgtggcggtaaacggcgaagcgttaagcccgttcggctgcctggacaagctgaatgcaattggtgcgcgtcacggcgtgggccgtatcgacatcgtggaaaaccgcctggtgggcattaagtcgcgcggctgctacgaaacccccggtggcaccattatggtcaacgcgctgcgtgcggttgaacagctggttctggatcgtgacagctttaaatggcgcgagcagctggggctggaaatgtcctatgtggtttacgatggccgctggttcgcaccgctgcgtaaatcaatccaggcttccgccgaagcgctggctgaagaagtgaacggtgaagtggtgctgcaactctacaaaggtcaggttaccgccattcagaagaaatcagccaacagcctgtattccgaagagtttgccactttcggcgaagacgaagtgtacgatcaccgccatgcaggcggctttatccgtctgttctcactctcttcacgcatccgcgcgttgaacgaaaagaagtaa

SEQ ID NO: 8 - DNA, 1374 bp, Erwinia amylovora
  source 1..1374: mol_type = unassigned DNA
atggcactttggggtggacggtttacgcagccagcagatcaacgtttcaagcagtttaatgactcactgcgcttcgactatcgtctggcagagcaggacattatcggctccgtcgcctggtccaaagcgctggtaacggtcaatgtattaacccacgcggagcagcagcagctggaaagcgcgctgaatgcgctgctggatgaagtgcgcgccgatccgcaacagattctggccagtgatgccgaagatattcatagctgggtggaaggcaaactgattgataaagtcggcgcgctgggtaagaaacttcacaccgggcgtagccgcaatgaccaggtcgcgaccgacctgaaactgtggtgcaaagcgcaggtcagcgagctgctggccgccacgcgtcagttccagcaggcgctgatcgccaccgcggaagccaatcaggatgcggtgatgccgggttatactcacctgcaacgcgctcagccggtcacctttgcacactggtgcctggcctatgttgagatgctggcgcgtgatgaaagccggctgcaggatacgctgaaacgcctggacgtcagtccgctgggctgcggcgctctggcgggaaccgcttatgaaatcgatcgtgagcagctggccggctggttaggttttgcttctgctacccgcaacagccttgataccgtatctgaccgcgaccatgtgctggaactgctgtccgatgcttccatcggcatggtgcacctgtcgcgcttcgcggaagatctgattttcttcaataccggtgaagccgggtttgtggagctgtccgacaaggtgacttctggctcctcgctgatgccgcagaagaaaaacccggacgcgctggagctgatccgtggcaaggttggccgcgtacagggtgctctgacggcgatgagcatgacgctgaaaggcctgccgctggcctataacaaagatatgcaggaagacaaagaagggctgttcgacgcgctcgacacctgggctgactgcctgcatatggcggcgctggtgctggatggcattcaggtgaaacgtccgcgctgtaaggaagcggccgagcagggctatgccaattccaccgaactggcggattatctggtggctaaaggcgtgcccttccgtgaagcgcaccatatagttggcgaagcggtagtggaagcgattaaacggggcgtggcgctcgaagcgctgaagctgtcagaattgcagcagttcagtcgcgtcatcggcgacgatgtttatccgattctgtcactgcaatcctgtctggataagcgtaacgcgaaaggcggcgtgtctttgcatcaggtatcgcaagcgattaccgaagccaaacggcttctggcttaa

SEQ ID NO: 9 - DNA, 1011 bp, Erwinia amylovora
  source 1..1011: mol_type = unassigned DNA
atgaatccgttatatcaacgtcactttctcaggctgctcgatttcagcccctctgaaatcaatggcttactggcgctggcatcccgcctgaagttcgctaagaaaaagggcgaagagattcaatatctgacaggcaaaaacattgcgctcatcttcgaaaaagactcgactcgtacccgatgctctttcgaagttgccgcattcgatcagggcgctaacgtcacctatcttggcccaagcggcagtcagatcggccataaagagtcgattaaagacaccgcacgcgtgcttggccgcatatatcacgccattcagtatcgcggacacggccagcagctggtggaatcgctggccgaacacgctggcgtgccggtatggaacgggctgaccaatgaattccaccctacccagctgctggccgacctgttgacgatgcaggagcatttgccggggaaagcctttagtcagatgacgctggcctatgtgggtgatacgcaaaacaatatgggtaacaccctgctggaagcagcagcgctggtcggcctcgatctgcgcctggtggcaccaaaaagctgctggccggatgcagcgctggtggccgaatgccgggatgtggcacagcacaacggcgggaaaatcaccctgacggaagatattgccacaggggtacaaggcgctgattttatttataccgacgtctgggtttcgatgggcgaacccaaagaagtatggcatgaacgcattgccctgctgcgtccgtaccaggtgaatatggcgctgctgcaggcgacgggaaacccccgggtgaagttcctgcactgcctgcccgccttccacgacgatcagaccctgctcggtcagcagatggcgcagcagtacgatctgcacggcggcatggaagtgaccgatgaagtgtttgaatccgcgcacagcgtggtgttcgatcaggcggaaaaccgtctgcataccatcaaggcggtgatggtcgccacgctgtcaaagcaggagtaa

SEQ ID NO: 10 - DNA, 1254 bp, Erwinia amylovora
  source 1..1254: mol_type = unassigned DNA
atgcttgaacaaatgggccaggccgcgaaggccgcctcttatcagctggcggtgctctccaccgcagagaaaaatcagttactgatgacgattgccgaccgtctggaagcgcagagcgcagagatcctcgcggctaaccaactggatcttgccgacgcgcggcaaaacggcatgagcgaggcgctacaggatcgtctgatgctggatcggcaacgtctgaaaggcatcgccaatgacgttcgtcaggtgtgccgcctgcccgatccggtcggagtggtgattgacggcggtaagctcgatagcggactgcgcattgaacgccgtcgcgtgccgctcggcgtggtcggggtgatttatgaggcgcgcccgaacgttaccgtggatgtcgccagcctgtgcctgaaaaccggtaatgcggtgatccttcgcggcggcaaagagacctaccgcactaatgctgccaccgtgcgcgttattcaggatgcgttgaaacagtacggcgtgccggctggtgcggtgcaggcgattgaaagcccggatcgtgcgttggtgactcagctgctgaagctcgatcgttatgtcgatatgctgatcccgcgtggtggtgccggactgcacaagctgtgccgagagcagtcaaccataccggtgatcaccggtggtattggcgtttgccatatttacgtcgatcgcagtattgagccggaagcggcgctaaaggtgatcgttaatgccaagaagcagcggccgagcgcctgtaactcgttggaaaccctgctgattgatgcggcgatcgccgacagtttcctgccagcgctcagcgcgcgcatggcgcaggagggggttagcctgcacgccgatgtgcgctcgctgccgctgttgcagcagggtcctgccagcgtcagcgcggttaccgatgcccagtaccgcgacgaatggctggcgctggatctgaacgttaaactggtggatgacctcactgaagcgattgcgcacatccgcgaatacggcacccagcactccgatgccatcctgacacgcaccattaagcatgcggagcgcttcgtcaatgaagtggactcatctgcggtttacgttaacgccagcacccgtttcaccgacggcggacagtttggtttaggcgctgaagtggcggtcagtacgcagaaactgcacgctcgtggcccgatggggctggaagcgctgactacctataaatgggtggcgttcggtgacgataccgtgcggggttaa

SEQ ID NO: 11 - DNA, 813 bp, Erwinia amylovora
  source 1..813: mol_type = unassigned DNA
atgatggaaaagaaaattggttttattggctgcggcaacatgtccaaagccattattgctggcctggtttcggccggacagtttaaagcagaaaatatctgggtctttgaccataaacccgccactaatcaggccatgcagcagcagtacggcattactccggcagaaagcgcccgccagctggcggaacaggcggatattctgtttggcgcggtcaagccaaacaggatcctgtccgtgctgaaggatgtggccggcagcctcaacaaagagacgctggtggtctccatcgcagcgggcatcaccctggattcactggcatccgtgcttggccatgaccgtaaaatcgtgcgcgttatgccaaataccccggccctggtcaacgaaggcatgagttcggttaccccgaacgtgctggtcacccaagaagaagcggatgaagtggtaaacatttttagcggttttggcaaagcagcgctggtaccggaatacctgatccacgcggtcgttggcgtaagcggctccgcgccggcctatgtgtttatgtttatcgaagcgatggcagatgcggcagtgctgggcggaatgcctcgtgagcaggcgtatcagtttgcagcgcaatcggtaaaaggtgccgcgcagatggtactggaaaccggtaaacatccggctgaactcaaggatatggtgtgctcgccgggcggtactaccattgaagcggtaaaagttctggagcagaaaggcttacgttcagcggtgatcgaagccatacagcagtgtatgaataaatcagaacagatgagcaagcagtaa

SEQ ID NO: 12 - DNA, 2463 bp, Erwinia amylovora
  source 1..2463: mol_type = unassigned DNA
atgcgagtgctgaaattcggtggaacctcggtagccaatgcggaacgttttctgcgtgtggctgacattttggaaagcaatgcacaacagggacaggttgctaccgtgttgtccgcgccggcaaaaattaccaaccatctggtggcaatgattgagaaaaccattagcggccaggatgcttcagcgaatatcagcgatgcagagcgtattttcgccgcgctgttgcaggggctgaccgacgcccagccggggtttgcttatgaccgtctgaaagcggtggtggaccaggaattcctccagctcaaacaggtgctgcatggtattgcgctgctggggcagtgcccggacagcgtcaatgcggccattatctgccggggtgaaaagctctcaatcgccataatggaaggtgtgttgcaggcgcgcggccacggggttagcgtgattaatccggtcgagcagctgttggccatcggccattatctggaatccaccgttgatattgccgaatccacccgccgtatcgccgcccgccagatcccggccagtgatatggtgctgatggccggctttaccgccggtaatgaacatggagaactggtggtgctggggcgtaatggctcagattactctgcggcggtgctggcagcctgtctgcgcgctgactgttgcgaaatctggaccgatgttgacggcgtgtacacctgtgacccgcgtcaggtaccggatgcgcggctgctgaagtcgatgtcctatcaggaagcgatggagctttcctatttcggcgctaaagttcttcacccgcgcactatcgctcctatcgcccagttccagatcccctgtctgatcaaaaataccggcaatccgcaggcccccggaaccctgataggcggtaacggcagcaatgatgacaacccggtaaagggcatcactaacctcaacaatatggcgatgttcaacgtctccggcccggggatgaagggcatggtcggcatggcggcacgcgtgtttgccgccatgtcacgcagcggaatttcggtagtgctgatcacccaatcctcctcggaatacagcattagcttctgcgtatcgcagagcgatctgccgcgtgcgcgtcgggtgctggaagaggagttctatctggagttaaaaaacggcctgctggaacctttggatgttatggagcagctggcggtgatttcggtagtgggcgatggtatgcgcactctgcgcggcatctccgccaagttcttttcagcgctggcgcgggccaacattaatatcgtcgctatcgcccagggatcgtccgagcgttccatctcggtggtggtcaacaatgatcaggcgatcaccggcgtgcgcgtggttcatcagatgctgtttgccaccgaccaggtgatcgaagtctttgtgatcggcgttggcggcgtgggtgctgcgctgatcgaacagctgcatcgccaacaaatctggttgaaaaataaacacatcgatctgcgtgtctgtggcatagccaactcgcgggcgttgctgactaacgtgcacggcatcccgctggatagctggcaggatcggctaaaagcggcgcaacaaccgtttgatcttgggcggcttaatcgcctggtgaaagagtaccacctgctcaacccggtgattgtcgactgtacttccagccagcaggtggcggatcggtacgctgatttcctcgctgacggcttccatgtggtgacgccgaacaaaaaggccaatacgtcatcgtacaattactatcagcaaatgcgaacggcagcggcaaaagcgcgccgcaaattcctctacgataccaatgtgggtgccggtctgccggttatcgagaacctgcaaaatctgctgaatgcgggggatgaattgatcagcttctccggcatcctttccggttccctgtcgtttattttcggcaagctggatgaaggcgtttcgctgtctgatgccacccaaatggcgcgtgaaatgggctttaccgagccggatccgcgtgacgatttgtccggtatggacgtggcgcgtaagctgctgatcctcgcgcgcgaagcaggttatcagctggagctaagcgatatagaaattgaagcggtgctgccgccagagcttaccgccattgccgatgttgacgagtttatggcgcgtttgccggagctgaacgaaggctttgccgcgcgggtgtccagcgcccgtgatgccggaaaagtgttacgttttgttggtgccattgaggaaagcggggcgtgcagggttaaaatcgctgctgttgacggtaacgatccactgtataaagtgaaaaatggtgaaaatgcgctggcgttctatagccgttattaccagccgatcccactggtgctgcgcggttacggcgccggtaatgatgtgaccgcagcgggcgtatttgccgatctgttacgtacactgtcgtggaagttgggagtttaa

SEQ ID NO: 13 - DNA, 930 bp, Erwinia amylovora
  source 1..930: mol_type = unassigned DNA
atggttaaaatttatgcccctgcctcaattggcaatgtcagcgtcggtttcgacgtgctgggggcggcggtatcgccggttgatggcaccctgctgggcgactgcgtcagcgtggaagccgccactgagttcagcctgcataatgaaggtcgttttgtcagcaagctgcccgctgacccaaaagaaaatattgtttaccagtgttggcagcgtttctgcaccgccattggccagcacgtcccggttgccatcacgctggaaaaaaacatgccgattggctccggccttggctccagcgcctgctcggtggttgccgggctaatggcgatgaacgaatactgcaacaagccattaaatgataacgagctgctggtattaatgggcgagctggaagggcgcgtctccggcagcgttcactttgacaacgtggcaccctgttttcttggcggtatgcagctcatgctggaggaaaacggcattatcagccagccggtgccaggctttgatgactggctgtgggtgatggcctatccgggcatcaaggtgtcgacggcgcaagcgcgtgccatcttgcccgcgcagtaccgcaaagaagatattattcgccacggtcgctatctgggcggctttattcacgcctgccatacacagcagccgctgctggccgcgaagctgatgcaggatgtagttgctgaaccttatcgcaccaaactgttaccgggctttgcccaggctcgccaggcagctactgatattggcgcgctggcatgcggtatttccggctccgggccaacgctgtttgccgtttgtaatcagccggataccgcaaaacgtatggcagactggctgggtcagcactatctgcaaaatgaggaaggcttcgttcatatctgccgtttagacacggcaggcgcacgtaaactgggataa

SEQ ID NO: 14 - DNA, 1191 bp, Erwinia amylovora
  source 1..1191: mol_type = unassigned DNA
atgacacgtttaaatccttattttggtgagtttggcggcatgtatgtgccgcagatcctgatgcctgccctgctgcagctggaagatgcttttatcagcgcgcaaagcgatccggcatttcaggctgaatttaccgacctgctgaaaaattatgctggccgtccaaccgcgctgacactgtgtaaaaacctgaccaaaggcagcaaaacgcgcctttatctgaagcgtgaagatctgctgcacggcggcgcgcacaaaaccaatcaggtgctgggccaggcactgttggccaagcgcatgggcaaaaacgaaattattgctgagacgggtgccggtcagcatggcgttgcctcggcgctggcctgcgcgctgcttggcctgaaatgccgtatctatatgggtgcgaaagatatagagcgccagtcgcctaacgtatttcgtatgcgcctgatgggtgcggaagtgatcccggtgcacagcggtgctgccacgttgaaagatgcctgtaacgaggcgctgcgcgactggtccggcagctacgaaaccactcactatatgctgggtacggcggctggccctcatccgttcccgaccatcgtacgcgaattccagcgcatgattggtgaagagactaaaaagcagatcctgcaacaggaaggccgcctgccggatgcggtcatcgcctgtgtcggcgggggttctaacgccatcggcatgtttgctgactttatcgatgaatccggcgtggagctgattggcgtggaaccgggcggtcatggtattgaaagcggcgaacatggcgctccactcaagcacgggcgagtcggtatttattttggcatgaaatcaccgatgatgcagacatctgacgggcagattgaagaatcttactcgatttcagccggtctcgacttcccttccgtcggcccgcagcatgctcacctcaacagtatcggccgggcgaactacgtatcaattaccgatgacgaagcgctggatgcctttaaagcgctgtgccgcagcgaggggattatcccggcgctggaatcctcacatgcgctggcccatgcgctgaaaatggcgcgcgaaaatcctgaaaaagagcagctgttagtggtcaatctttccggccgtggtgacaaagacatcttcaccgtgcacgacatccttaccgctaagggagaaatctga

SEQ ID NO: 15 - DNA, 1362 bp, Erwinia amylovora
  source 1..1362: mol_type = unassigned DNA
atgcagcaaaccgtacttgatcgcatcgtgcgcgataaaaccgactggctggcggctcgccagcagcagcagcctctggcctcgtttcagcatgagattgtcccggctacgcgcaacttctacgaggggctgaagggcgcccgtaccgcgttcattctggagtgcaaaaaagcctcgccgtccaaaggattgatccgtgaagactttgacccggcggtgattgccggcgtctaccggcattacgcctccgccatctcggtgctgaccgatgagaagtactttcagggaagctttgactttctccccatcgtcagtgcagcagtcagtcaaccggtactgtgtaaagactttatcatcgacccgtatcagatccatctggcgcgctattatcaggccgacgccattctgctgatgctgtcggtactgaatgacgagcagtaccgccagctggccgccgtggcacacagcctgaatatgggcgtgctgacggaagcgatcagcgatgaagagcttgagcgcgccaccgcgctgggcgcaaaagtggtcggcatcaataaccgtgatttacgcgatctgtcaatcgacctcgaccgtacccgccgtctggcgccgaaagtggcacagggcgtgacggtcatcagtgaatcaggcatcaacagctatgcccaggtgcgcgaactgagtcacttcgccgacggcttcctgataggttcggcgctgatgtcggaagacaaccttgccgctgcggtcaggcgggtgattatcggtgagaacaaagtctgcggactgacccgcccgcaagatgcgcgcagcgcgctggagtgcggggctatctatggcgggctgatctttgccgccggatcgccgcgccagattgacgccgcacgggcgcaagaggtcatggccgccgccgcactcaggtacgttggcgtgttccgtaatgacggcctgagtgacgttgtcactatcgcgcggcggctcaacctgcacgcggtacagctgcacggtgatgaagatcgcgagttcattagccagctacgagccgcattgcctgaaaacgtgcagatctggaaagccttcagcatcagcaccagcctgcccgctcgcgactggccgcacgtcgatcgctacgtatttgaccagggaaatggcggcagcggtaaagcgtttgactggtcgctgttacaaggtgaaaagtgggataacgtcctgctggccggcggcctgagtgcggacaactgccttctggccgcacagcagggctgtgccggtctcgactttaattccggcgtggagagcgctcccggaatcaaagatgcagcaaaaatggcttccgtgttccatacgttgaaagcctactga

SEQ ID NO: 16 - DNA, 1563 bp, Erwinia amylovora
  source 1..1563: mol_type = unassigned DNA
atgccaaatgccaaacctggagtgaaattgatcaccgccagtgcgccgtaccgggaagatcccgctgcggttttccatcagctctgcgcggctcgttcagaaacgctgctactggaatctgccgacgtcgacagtaaatgcaatctgaaaagcctgctgattgttgacagcgctctgcgcatcagcgcactggacaatcgtgtgcagatccaggcactgtcggccaatggccgtgcactgttgccattgctggatgccgcattgcccttatccgtggtcaaccatgtgggtactgatagccgtgagctggtgtttccgcacaatgaggagattcaggacgaagacgctaaactgcgcgccctgtcagtatttgatgcgctgcgcctgatccctcaactggtaaattctccgcaccaggagcgtgaagcaatgctgcttggtgggctgttcgcctatgacctggtcgccggcttcgaaacgctgccggagattgccaaccagcagcgctgcccggactactgcttctatctggctgaaacgctgctggttatcgaccaccaggcacggagcgctcgcctgcaggccagcctgtttaccccgaacgcgtctgagttccagcgtttgcagagccgcgtagaacagctgtgcggcatgatgtctcaacccgtcagcgccttaccggtgcaaacggtggacagtatgcagcttagcgtcagtcagagcgatgaagaatactgcgccgttgtgcgccagatgcaggaagcgatccgcgtaggtgaaattttccaggttgtgccttcgcgccgtttctctttgccttgcccttccccgctggccgcttacgacacgctgaaaaacagtaaccccagcccgtatatgttctttatgcaggatcgcgacttctgcctgttcggcgcctcgccggaaagttcgctgaagtacgaagccgccagcaatcaaatcgaaatctatccgattgccggaacgcgcccgcgtggccgtcacgccgatggctcgctcgatcgcgatctcgacagccgtattgagcttgaaatgcgtaccgatcataaagagatggccgaacatctgatgctggtcgatctggcgcgcaacgatttagcacgtgtctgccagcccggtactcgctatgttgccgacctgaccaaagtcgatcgctacaccttcgttatgcacctggtctcacgcgttgtcggtcagctgcgtaatgaaatagatgtgctgcacgcgtatcgcgcctgtatgaacatgggtacgctgagcggtgcgccgaaagtgcgcgccatgcagctgattgccaaagccgaaggcacacggcgcggcagctatggcggcgcggtcggttactttaccgccagcggcgatctcgacacctgtattgttatccgttcggcttacgttgaagacggtatggcaacagtacaggccggcgccggggtggtactggactccaacccgcaagcggaagcggatgaaagccgcaataaagcccgcgcggttctgcgcgccatcgcctctgctcatcactgccaggagattttctga

SEQ ID NO: 17 - DNA, 594 bp, Erwinia amylovora
  source 1..594: mol_type = unassigned DNA
atggccgatattctgttgcttgataactttgactcattcacttacaacctcgtcgaccagctgcgtacctttggccaccaggtgacgatttaccgcaataacgtcccggcagagacgctgattgcttgtctgcaacagatggaaagtccggtgttgatgctgtcgcccggtcccggcgcccctgcagacgcgggctgtatgccagaactgctccggcagctacgaggccgcctgccgatcattggtatctgtctggggcaccaggcgatagtggaggcatacggcggccacgtcggtcaggcgggggaaatcctgcatggcaaggcctccgccatcagtcatgacgatcacggcatgtttgccgggctgagcaacccgctgccggtggcgcgctaccactcactggttggcagcgcgatccccgccggactgaccgttaatgccacttttaacgatatggtgatggcggtgcgcgatgacgctaatcgcgtctgcggcttccagttccatccggaatccattctgaccagccagggcgcgcgtctgctggagcaaaccctcgcctgggcgctggcgtcatcaaggagccattga

SEQ ID NO: 18 - DNA, 714 bp, Erwinia amylovora
  source 1..714: mol_type = unassigned DNA
atgcaaaaaaaagctgagttgtatcgcggcaaagcgaaaaccgtttacagcaccgaaaacccggatctgttggtactcgaattccgtaacgatacgtcagcgctggacggtgagcgtatcgagcagtttgatcgtaaaggaatgattaacaacaagttcaatcatttcattatgactaaactgcaggaagccgggatcccaacacagatggaggccctgctttctgataacgaagcgctggtaaaaaatctggatatggtgcccgtagaatgcgtaatacgtaatcgtgctgcgggatccctggtgaagcgtctgggcgtggaagagggcatgatcctcaatcctccgctgtttgatctgttcctgaaagatgatgctaaacacgatccgatggtcaacgaatcctattgcgagacgttcggctgggtcagcaaagcaaatctggcacgtatgcaggagctgactttccaggcgaatgacgtcctgagcaagctgtttgccgatgcaggcctgatcctggtggacttcaaacttgagttcggcctgtttaaaggcgaagtggtgctgggcgatgagttctcaccggatggtgcgcgcctgtgggataaagacaccctgaataagatggacaaagatcgttatcgtcagagccttggtgggctgattgaagcctatgaagaggtggccaaccggttgggcgtgaagctggactaa

SEQ ID NO: 19 - DNA, 936 bp, Erwinia amylovora
  source 1..936: mol_type = unassigned DNA
atggccaatccgctatatcgcaaacatattatttccattaacgatctcaaccgtactgagctggagctggtgctgaacaccgccgccagcctgaaagcggccccgcagccggaactgttgaagcataaagtgattgccagctgcttctttgaagcctcaacccgcacccgcctgtcgtttgaaaccgccatgcaccgcctcggggcgtcggtggtgggctttgccgacggcggtaatacctctctggggaagaaaggcgaaaccctggccgatactatttcggtgattagcacctatgtggatgccatcgtgatgcgccatccacaagaaggggccgcccgactggcaaccgagttttccggcggcgtgccggtactgaacgcgggggacggagccaaccagcatccaacccagacgctgctcgacctgtttaccattcaggaaacccaggggcgtttgagccatttgcaggtcgcgatggttggcgacctgaaatatggtcgtaccgtgcactcgctggctcaggcgctggcgaaatttgacggcaaccgcttctggtttatcgccccggatgcgttggcgatgccggcttatatcaccgatatgctggacgaaaaaggcattgcctggacgcgccacgacagcattgaggaggtagtgccacaggtggatattctgtatatgacgcgcgtgcagaaagagcggctggatccctcggaatatgccaatgtcaaagcgcagtttgtgctgcgcgctgcggatttgaacggtgcgcgtgacaatatgaaggtgctgcatccgctgccgcgtattgatgaaatcgacaacgacgtcgacagcacgccgcacgcctggtatttccagcaggctggcaacggcatttacgcacgccaggcgctgctggcgctggtactcaacagtgaaccggctctgtaa

SEQ ID NO: 20 - DNA, 1467 bp, Erwinia amylovora
  source 1..1467: mol_type = unassigned DNA
atgctaagaatcactaaagaagccctgacattcgacgacgttctgctcgttcctgctcactccacggtgctgccaaacaccgccgacctcagtacccagctcactaaaaacatccgtctgaacattcctatgctgtctgctgctatggacaccgtgaccgaagccggtctggccattgcgctggcacaggaagggggcctgggctttatccacaaaaacatgtctattgagcgccaggcggaagaagttcgccgggtgaagaagcatgaaagcggcgtggttaccgacccgcagaccgtgttgcccactaccgcgcttagcgaagtgaaagcattgaccgaacgtaacggctttgccggttacccggtggtcaacggcgaaaacgagttggtcggtattattaccggacgtgatgtccgctttgtcaccgatctgagcctgccggtttctgcggtaatgacgccaaaagagcgtctggtcacggtgaaagagggggaagcgcgcgaagtggtgctgcacaaaatgcacgaaaaacgcgtggaaaaagcgctggtggtggatgacagtttccatctgctgggcatgatcaccgtgaaagacttccagaaagcggaacgtaaaccccttgcctgtaaagacgagcatggccgtctgcgcgtgggtgctgccgtcggtgcaggtgccggtaacgaagagcgcgtcgatgcgctggtggctgccggtgttgacgtactgctgatcgattcctcccacggtcattctgaaggcgttttgcaacgcatccgcgaaacacgtgctaaataccccgatctgcaaattgtcggcggcaacgtggcgaccggcgctggcgcgctggcgctggccgaagccggcgtcagcgcggtgaaagtcggtattggtcctggctcaatctgtactacccgcatcgtgaccggcgtgggcgtaccgcagatcaccgcagtttctgacgccgttgccgcgctggaaggcaccggcattccggttatcgctgacggcggcatccgcttctccggcgatatcgcaaaagctatcgccgccggtgctgctgcggtgatggttggctccatgctggcaggtactgacgagtcgccgggcgaaatcgaactgtaccaggggcgttcatacaagtcttaccgtggcatgggctcgctgggcgcgatgtcgaaaggttcttccgaccgttacttccagaccgataatgcggcggacaaactggtgccggaaggcatcgaaggtcgcgtggcatacaaaggtcgcctgaaagagatcgtgcatcagcaaatgggcgggctgcgctcctgtatgggcctgaccggctgtgctactatcgacgagctgcgcaccaaagcgcaattcgtgcgcatcagcggtgccggtatttcggaaagtcacgttcacgatgtgaccattactaaagagtcgccgaactaccgtatgggttcctga

SEQ ID NO: 21 - DNA, 996 bp, Erwinia amylovora
  source 1..996: mol_type = unassigned DNA
atgactatcaaagtaggtatcaacggttttggccgtatcggtcgcatcgttttccgtgctgctcaggaacgttctgacgtagaaatcgttgctatcaacgatctgctcgacgcagattacatggcttacatgctgaagtatgactcaactcatggtcgctttaacggcactgtagaagtcaaagacggccatttggttgttaacggcaaaaccatccgcgttactgctgagcgcgatccggcgaacctgaagtgggatgcggttggcgttgatgtggttgctgaagcaaccggtatcttcctgaccgatgaaactgctcgtaaacacatcgaagctggcgctaagaaagtggtactgactggtccgtccaaggatgataccccgatgttcgtaatgggggttaaccacaaggcttacgcgggtcaggctatcgtttctaacgcatcctgcaccaccaactgcctggcaccgctggcgaaagtgatcaacgataaattcggtatcgttgaagcattgatgaccacggtacacgccactacggcgactcagaaaaccgttgacggcccgtctcacaaagactggcgcggcggccgtggcgcatctcagaacattatcccttcttctaccggtgcggctaaagcggttggaaaagtgatccctgagttgaacggcaagctgaccggtatggcgttccgcgttcctacacctaacgtttccgttgttgacctgactgcacgtctggaaaagcctgcttcttataaagaaatttgtgacacgattaaagcggctgccgaaggtgaattgaaaggcattctgggctacaccgaagatgaagtggtttctaccgattttaacggcgaaaagttgacctctatctttgatgcaaaagccggtatcgccctgaacgataactttgtgaaactggtatcctggtacgacaacgaaacaggttactcgaacaaggtgctggacctgattgctcacatcgctaaataa

SEQ ID NO: 22 - DNA, 1728 bp, Erwinia amylovora
  source 1..1728: mol_type = unassigned DNA
atgatttcaggaattttagcatcaccgggtatcgctttcggcaaggcgcttctgctgaaagaagatgagattgttattgaccggaaaaagatttctgccgaccaggtcgatcaggaagttcagcgttttcttgagggtcgtaaaaaagctgccgcacagctggaaacgattaaaaccaaagcaggtgaaacctttggcgaagaaaaagaagcgatcttcgaagggcacatcatgctactcgaagatgaagaacttgagcaggaaatcatcgctctgattaaagatgagcacgccaccgctgattctgctgcctattctgttatcgatggccaggccaaagcgctggaagagctggatgacgagtatttgaaagagcgtgctgccgacgtccgtgacatcggtaagcgcctgctgcaaaatattcttggcctgcatattgtcgatcttagcgccatcaatgaagaagttctgctgattgccaaagatttgactccgtctgaaacggctcagctcaacctgaaaaaagtattaggtttcattaccgatattggcggccgtacttctcacacctcgatcatggcacgctcgctggagctgccagccattgttggtaccggtgacgtgaccagccgggtgaaaaacgacgatttcctgatcctcgacggcgtaaataaccagatatacatcaacccgacgccggaaaaaatcgaagaattaaaagccgtcgcgcatcagtacgtcagcgagaagaacgagctggcaaaactgaaggacctgccggcaatcacccttgatggtcatcaggttgaagtgtgcgcaaacatcggcaccgtacgcgacgtcgcgggggctgaacgtaacggcgctgaaggcgttggcctgtaccgcaccgagttcctctttatggaccgcgaatcgctacccagtgaagatgagcagttccaggcctataaagccgtggcggaagcggttggcgctcaggccgtcatcgtgcgaactatggacattggcggtgataaagatctgccgtatatgaacctgccaaaagaagacaacccgttccttggctggcgcgctattcgcatcgcgatggatcgtcctgaaatcctgcatgcacagctgcgtgctatcctgcgcgcctctgcctttggcaagctgcgcatcatgttcccgatggtgatttccgttgaggaagttcgcatcctgaaagcagagctggaaaccctgaaagcccaactgcgtgcggaaggtaaagcattcgacgagaaaatcgaagtgggtatcatggtggaaacgcctgcttccgcagcgattgctcatcatctggcgaaagaagtcgactttttcagtattgggactaacgatctgactcagtacactttggcagtagatcgtggtaatgatctgatttcgcatctctacaatcctatgtccccttctgtattaacgttgattaagcaagttattgatgcttctcacgctgcaggtaaatggaccggaatgtgtggtgagctggcaggtgacgaacgtgctacactactgttactgggaatggggctggacgaatttagcatgagtgccatttctatcccacgcatcaagaaaattattcgtaatacccattttgaagatgcgaaggcgttagcagatcaagctctggcccagccgacagtggatgaagtgatgaacctggtgaataagttcattgaagaaaaaacactctgctga

SEQ ID NO: 23 - DNA, 1410 bp, Erwinia amylovora
  source 1..1410: mol_type = unassigned DNA
atgtcagctgaacacgttctgtcgatgatgaacgagcacgaagtcaaattcgttgacctgcgtttcaccgataccaaaggcaaagagcagcacgtcactatccctgctcaccaggttaatgctgacttcttcgaggaaggcaaaatgttcgacggctcctcgattggtggctggaaagggattaacgaatccgacatggttctgatgccggatgccaccaccgctgtcctcgatccgttcttcgaagatgccaccctgattattcgttgcgacatccttgagccaggcaccatgcagggttacgaccgtgacccacgctccatcgccaaacgcgcagaagatttcctgcgctcttccggcattgccgacaccgtgctgttcgggccagaacccgagttcttcctgtttgacgatatccgtttcggcagcagcacttccggctcccacgttgctatcgatgatatcgaagccagctggaacaccggtaaacaatacgaaggcggcaacaaaggccatcgtccggggctgaaaggcggctacttcccggttcctccggtcgactcatcacaggatatccgttctgcgatgtgtctgacgatggaacagatgggcctggtggttgaagctcatcaccacgaagtcgccactgccggtcagaatgaagtggctacccgcttcaacaccatgaccaagaaagctgacgaaattcagatctacaaatatgtcgtccacaacgtggctcacgcttatggcaaaaccgccacctttatgcctaagccaatattcggtgataacggctccggtatgcactgccacatgtcactgtcaaaaggcggaactaacctgtttgcgggcgacaaatacggcggcctgtctgaaactgccctgttctacatcggcggagtgattaaacacgctaaagcgatcaatgcgctggctaacccgaccactaactcctataagcgtctggttcctggctacgaagcaccggttatgctggcttactctgcgcgtaaccgttccgcgtctatccgtattccggttgtcgccagcccgaaagcacgccgtattgaagcgcgcttccccgatccggctgctaacccgtacctgtgcttcgcggcactgttgatggctggccttgacggcatcatcaataagatccacccgggcgatgcgatggacaaaaacctgtacgacctgccgccggaagaagaagcggagatccctaaagttgctgcctccctggacgaagctctggccgctctcaacgaagaccgtgagttcctgacgcgtggcggtgtgttcaccgacgacgcgatcgaagcttatatcgagctgcgtaaagcggagatggagcgcgtgcgcatgaccccgcacccggttgagttcgaactgtactacagcgtttaa

SEQ ID NO: 24 - DNA, 5535 bp, Erwinia amylovora
  source 1..5535: mol_type = unassigned DNA
atgtccaactcaacaccttttccttacggtttgtacgatccctcaaaagacagcgacagctgcggcgtgggcttcattacgcgtaaggatggcgcgcaaactcatgaggtgctggaaatggcacacggcgctctgtgcaccgttccgcatcgtgggggaatgtcagccgaaggcgtgggtgatggcgccggggttaacgtagacctttcactgcacttcttccgtaaaatcaccggccagccgctggaagctggccgtttcggcgtcggtaacttctttgtgccgaaagacagcacgctgcgcgccaacgccgagcggctggttgaagacacgctgctttcctgtgacctgaaaattattgccaggcgtgatatcccgctggacaacagcgtgttgcgcccggcagcgatccctttccagctgcctgttgcgcaatggatatttgctgccccggacgatgtcagcaaccagattgatttcgagaagcgcatttatcgcgccctgctgcaaatcgaagcccgtgcttttagcgacggcgaattcggcgggctttacccgctgtcgctctcctcgcgcacccaggtgttaaaagcacgcctgaactcgaatgaagtgatcccctatttcaaagatctgaccgatcccgatcatcaggtacgcggtctgttcttccatacgcgcttctcaaccaataccgatccgcataccaccatggcgcagccgttccggctgatggcgcataacggcgagctaaacaccgaccgtaaaaaccgcattgcggaagccgcgctggcgctggcgcgcggtaagaaaatcgtgcgcccgaaagggcaatcggacagttcacgtcttgaccagagcatccacagccgcctgatggaagacaatctcgacctggtcaccgccgtggtttccatgatgccgcccgcctgggaaaacgatccgtcactgtcgcccgaagtacgtgccatgctggagtatttctcgctgtatgaagagaagaacgacggcccggccgcgctgatttttggcaatggcgaagtgatcggcgcacgtctcgaccgtcttggtctgcgtccactgcgctcggtggaaaccgccgaatatatcggtgccatgtcggaagctggccagatcgccttcccgccggaaagcgtgctgcgccgtggccgtatcgaagcaggcggcatgctgtattacgatcaccgcgaaaagcgcagttacaccaccattgacgcgctggaaaaactggcggcggcggctgactatccggccttgctggcgaagtcccgcgtgctgttgcaggatctgccggtggttccggctgaacagcagggatctccgttgcgttatggcggcgacctcaaaacctggcagcgttttgtcgcctattactataaccaggaaagcttcaaatttatgatggacccgatgctgaccaccggcgcagagaaaatctccgctatgggttacggcaacgccatcaacgccctgtccgaccatgaaggcggcatggcgcactatttctcccagcgcttcgcacaggttaccaacccgccgctggactccatccgtgaagctgacggcatgactctgcgggttgcacttggtgccaaaccgcatttgggtcgcagcaagggccagcagatcgtgatccctacgccgatcctcacgcatcttgatatgctgcaactgcgcgagcagactgtggccccttatgcccgctttgaaatgctctatacgccggtcaccggcagcgatgccgcaagccgggcggccaatgccaacgcgctggagcacgccattgatgaactggcgcagcaggtggttgacttcgcccgtgaacagggtggcatcgcggtggtaaccgaccgccatctctccaccacgcgcgccgccatcccgatgctgctggtggtttctgccattaaccagcggctggtgcaggaaggtttgcgtctggacgtatcgctggtggttgagagcggccagagcatctcttcccaccatatcgccgcagcgctgggctttggcgcttctgccatctatccactgggcgtgcagatgcgtgccgaagagaaatatggcgagggtgaaggcggcaataaagccttcaaacgctatgccaaagccgctgagaaagcgctgatgaaaaccatgggcaaagtcggcctgtgtaccgtggaaagctacagctgcggcgagttctttgagcccaacttccttgataccgaagaccgcgtgctgaagaagtacttccccaacatcaaaaccccggttggcggcgctggctttgcctccatcgcgcagatggcggttgactggcaccagagtgcgctgcgcgtgcagggtgaaaatgacgttccgctactcggcctgttcaaagagcgggcagaaggcgcgggtcattcctacggcaccattgcggtgcgtacctttattgatatgacggagcagcctgtccgctttgccgataagccgcgcgaggaggataacttcatccgcctgatgacgctggcaaaactggataatgcgtttaatatcaaggcaaaatcctttgctgacagcagttttgaacgtatcccgaacgaggttatcgataacttttccattaccgctgactatcgccagttctccagcctgatgctggaagagcgcaaacgccgcccggcggcgctgcgcgatattcttgctttcccggcagatttaacccacgtcgacagccaggcggaatttacccgcaagctgggccgttattcgctggttaacaacggctttgcggtgcgcggcctggaatgcgaggccgtggcagaatgtgcaggccagttcaggctgcgcctgaccgatgcggtcgccggactgaaaagcgagcaggaacgcctgaccacgctgtgcggcgtgctgaccaaacgcttcggtgacgctatcgaacgcgccgaggtgacggcggatgcgctgctgcttacggccagtggtaaggcggctgagtatctggcgcgcatcttcactattgctccggcattgccgctaagcgaagtgcagccggcccatgaaattacccgcaccttcgcctccggggcgatgagccacggcgcgctggtcgctccggcgcacgaagcggtggcgcacggcaccaatatggtcggcggcatgagcaactgtggcgaagggggcgagcactactcgcgtcacggcaccctccgcgcatcacgcattaagcagctggcatccggtcgtttcggcgtctgggccgcctatctggccgacccgatgctggaagagctggaaattaagattggccagggggcgaagcccggcgaaggcggccagctgccgtcggccaaagtaacggtagaaatcgccgccgcgcgtggcggtacgccaggcgtggagctggtatcccctcctccgcaccacgatacctactcaatcgaggatctggcgcagctgatccacgactgtaaagccgcccgcgtgcgggtgatcgtcaaactggtttcctctgagggtattggcaccatcgccgttggcgtggccaaagccggcgctgacgtgatcaacgtggccggcaataccggcggcaccggggcagcttcggtaaccagcctgaaatacaccgggcgcgtggcggaaatcggcatcgctgaagttcatcaggcgctgtgtgctaacggcctgcgtgagaaagtcctgctgcgctgctccggtgcgcagcaaaccggcagcgatgtggtgaaatccgcgctgctgggcggcgacagcttcgagttcggtaccacggcactgatgatgctgaagtgcgtgatggcgaaaaactgcaatgtgaaatgtccggcggggctgaccactaacgccgaagcatttgacggcgacccgcgccagctggcgcagtatttcatcaacgtggcacacgaagtgcgcgagatgctggcgcggcttggcctgcgttcactgcgggaagcccgtggccgctccgacctgctgcacctgatggatcatccgctggaagtgggcaagctggatctgcgcgccatgctgacggtggttcccgaaatgaaaatcagccatccggtgtatctggaaaaagattttgcgctggataacggctggatcgatcggttacaggagcagctggtaatccagggatcgggcgcgatcgtactcggtgacggtatcaccctgaacaaccgcaataaaagcgtgggtggacagctggctatcgatatcgaacgcctgctcaatcaccagctgaacgccgaacatctgagccgcattccggctgcgctacaggacgatcgcggccgccgctatcttgccccggccacggtgaccgtcaccacctgcggttccgccgggcagtcttacggcgtgttctgtaatgacggcatgcgcctgcagcaccagggcacctgtaatgacggcgttggtaaaggccagtgcggcggcgagatcgtggtgagatcgcctggcggtggttcacaggacgcggaaggcaacgtgctgattggcaacttcgcgctgtttggcgctaccggcgggcgtctgtttgcccagggccaggcgggcgaccgctttgccgtgcgtaactcgggagcgacggcggtggtcgaaggcgttggtgatttctgctgtgaatatatgaccaacggcgcggtgctgaatcttggcacctgtggaacgggtttcggtaacggtatgagcggcggcttcgcctatcagtacgatccgtatggcacgctggcggattgcgccgccgccgactcggtaatgctcggtgccatcagcgatgacaatgaaatggcccgcgtgcatcagcaggcggtactgaccatgctgaactggcatctggaagcgaccggctcgccacgcgcccggtggttgctcgaccactgggaaacggaatgccagcattttgtttatgtgatgccgcgttctctgctgctgtatcaggacgcgactgaaatccttaaggcaaaagcgcgtaaggacctgctggaagaactttctaccgctctggccagtcaccaggtggcgaaattcaaagccgcatggcgcgccggcaaaccgatcgccgacggctcggttccggtctacggcgccatggacaccccggaaatgctggtgctgctgaataattataccgtcctgagctgtgcccagcagctggcgctgtcccgtctgccgaaaggcacggcggtgcaggacccggcggtggagaaagcggtgcgtaacctgctgatgacagaagattttgctctgattagtaaactgcagcgccacggtcgtgcggcgattgaaagctacagcgatgagcagctggcctgcctgatcggtgccaaacgcatggcggactataaagcggcgctgacgcagcgcaatatccgttcgatggacagtctggccacctatggctggatccagtatcaggatgcgtgtaacagggaagtgctgggacgactgcctgactttgaagagctgtttgcccgcgccgcactgcctgagttagcggcggcggtcggcaaactggccggataa

SEQ ID NO: 25 - DNA, 2778 bp, Erwinia amylovora
  source 1..2778: mol_type = unassigned DNA
atcgatcctttagattgttccgtcgagtatacggcagcattgtcaggcacagtggcatgcgcggaaaagcaaaaaccggccatcgcaaccgatgcagccgtaataataagtttcatattaactcccttatttgggtttgaattttgcttaatctacctggtcaaggcaaagtggtgaaacagtcctgtcccggcaagctaaaccagcgataagtatagccagtgtttaccgtttcatcgggcgctacagcagccccgccggattggtgtctgtgctgtccggctggaacacaatgcgaatcctggccttgatggcaaaacctgctgtgccagccggcgtatatcagcagcagatggggcaggtgctgcgggcctgattaatcgaggccgttaaagacgcttgtctctatctcgctgtattagaaataaatatggtcacgtcttgaaatacgacataaaaaaagataataattctcatttacatagtaaaatcggttacattcaggttatgcgcgcctgtctgctctggatccatccgcgaaataagagtaataagtctaatctttgtaagggaataaacatgagaatgccttttagcctcaagcgttctgtgctgctctgtggcatagcacttttcacgccgggtatgacctgggccgcagatacgctggtggttaccgctaaaccggaagaaaccgccagcacgccgacggaaggctacaccgcgaagatcagcagcggggcgaccaagaccgatcgcccattgatcaccaccggccagtccgtttccgtggtgactcgtcagcagatggaagatcagggggcgatggacgttaatcaggcgctgaattacaccgccggggcctttactaactttgctggcgcggcatcacgttatgatacggtatcgttgcgtggattccacggcggcgatgtcgacaacatttttgttgatggcctgcgcctgatgagcgatccgggtagcttcaatgcgttgcagattgatccgtggtttctcgacaggatcgacgtgatcaaaggcccttcttccgctctgtatggtcagaccgtgccgggtgggctggtgatggaaagctctaaacgcccgcagtttgcccctgaaggccatttccgcctgtcgaccggctccaacagtaccaacagcgcggcgtttgattacaccgatgcaatcaacgagcaatgggcgtttcgtctgaccggcattacccgtagcagtcatacccagtacgatcatacccgcgaagaaaaatacgccatttccccgtcgctgctgtggcagccggatgaggatacgtccctgctgcttcgtgcctatctgcaaaaagatccttccggcggttaccacagtgcggttccgggtgagggcagcattaccgcacacaacggcagaaaactgagcaccggtttttatgacggcgacagttcattggatcagtttaaacgccgggagcagatctacagcgctcaattctcgcaccgctttaacgacacctgggcgtttcgttccaacgccagctattcacattcaaatgtcgatctcgaccaggtttatcagattggctgggacactaataacgccgatctgttgaatcgttactattcaggatcgcgatcatcgctaaatgcctttgccatcgataatcagctggaagccgacttcgctaccgcggaagttgctcacagagtagtactgggagctgaatatcaccagtacaagaatgacctgagtgatgccagaggttcggccagccagctcaatgcccttaccggccaggtggtgggcagcaatgacaattttaccttctctaaatcgcagcgccgttactatcaggccggggtttatctgcaggatgaaatgacgtgggataaatggcatctggatctctccggccgctacgatcgcctcgttgcccggaccgacaacattgaccaggagactaaattccgtcgccaggacgaccacgtcggcgggcgcgcatccctgctgtatgcgtttgataacggtatttccccttatgccagctacagccaggcgattacgccgcagtctcttcctggcaaggacggcaatgtgctgaaaccgacgacttctgagcagtatgaagccggggtgaaataccagccggtgggaacgtcggatctctattccatcgccgtctacgatctgacgcaaaaagatgtcggcaaccgcgttattgtaggctcttattatgtcccagccgggaaagttcattcacagggcctggaactggaagcccacaaccagctgacgccgcgactcagtacccttgctacctacacgttgaatcacgtacgttataaggatgccattgatggcaatgacggccacaccccttacgttacgccaaattcgatggcttcagcctgggccaaataccagtttgactacgggattagcgtgggggccggggtgcgctacatcggcaagcagtgggcggataacgcaaacactacccgtctgccatcggccacgctatttgacgcgtccgttcgtgccgatctcggcgcctggaacagcagcctgaaaggggcctttgtgcaggtaaatgccaataacctgaccgatcgcgactacgtttctgcctgttacggtaccgggtattgttattggggcgctgaacgtacggtgatggctaccgtcggttacgatttctaacccgcgattcggtttacctgaataaaaagcacggccgtttaagcggccgtgcttttttttacccagaagaaggaggacgtt

SEQ ID NO: 26 - DNA, 1830 bp, Erwinia amylovora
  source 1..1830: mol_type = unassigned DNA
atgtgtggaattgttggtgctgtagcgcagcgtgatattgcagaaatcctgctcgaagggctgcgccgtctggaataccgtggctatgactctgccggtctggccgtggttgatgctaacgggcaggctacccgcctgcgtcgcgttggcaaggtgagcaacctgactgccgcggcggaaaacaccgcgctggcgggtggaaccggtatcgcgcacacgcgctgggccacgcacggcgaaccgtcggaaagtaatgcgcacccgcacatttccggcgaaatcattattgttcacaacggcattattgaaaaccatgagccactgcgcgagctgatgacaggccgcggttacacctttacctcagaaactgataccgaggtggtggcgcacctggtgcactgggaacagcaaaacggcggctcactgcatgaagtggtgcagcgtgtcatcccgcagctgcgcggtgcttacggtatggttatccttgacagtcgcgatcccgccacgctgatagctgcgcgctccggcagcccgctggtaataggccgtggcgtgggtgaaaactttattgcctcagatcaggtggcgctgctgccggttacgcgccgctttatctatctggaagagggtgatatcgcgcaaattacccgccgtaacgtcattatcgttgaccgcagcggcgaggttgtacagcgccgcgaaatcgaatccaatacgcagtatgatgccggcgataaaggtgcctaccgccactacatgcagaaagagatttacgagcagccggtggcgattaaaaacacccttagcggtcgtttcagccacggagaagtcgacttttccgagctgggcgccaacgctgactcgctgctggccaacgttgaacatatccagattgtcgcctgtggcacctcatacaactctggtatggtttcgcgctactggtttgaatctctggccaacgtaccttgtgacgtggagatcgcttctgaattccgctaccgcaaatctgcggtgcgcaaaaacagcctgctgatcaccctttctcaatccggtgaaaccgccgatacgctggcagcgctgcgcctgtcgaaagagctgggttatttgggatcgctggcgatctgcaacgtggcgggctcctccctggtgcgcgaatctgacctggcgttgatgaccaaagccggtacggaaatcggcgtggcatcaaccaaagcctttaccacccagctaaccgtcctgctgatgctggtggcaaaaattggtcgtcttaaaggcgttaatccacaggtggaacacgatattgttcactccctgcaggcgttgccgagccgtattgagcagatgctgtcacaggatgagctgattgcttccctggcagaggatttctcagacaaacatcacgcgctgttccttggccgtggcgatcagtacccgatcgccatggaaggggcgctgaagctaaaagagatctcctatattcacgctgaagcctacgccgccggagagctgaaacacggcccgctggcgctgatcgatgctgatatgccggtgattgttgtcgcgccgaacaatgaactgctggaaaagctgaagtcgaacattgaagaagtgcgcgcccgtggcggcctgctgtatgtcttcgccgatgcggatgccggttttgccaacagcgaagggatgcgtatcattccgctgccgcatgtcgaagaggtgatcgcgccaatcttctacaccgtgccgttgcagctgctcagctaccatgtggcgctgattaaaggcaccgatgtagaccagccgcgcaacctggcgaaatcggtgacggtggaataa

SEQ ID NO: 27 - DNA, 1935 bp, Erwinia amylovora
  source 1..1935: mol_type = unassigned DNA
atgtctatcgttgaaaaaaaacccacccgccgcgaacagcgcgcccaggcgcagcagtttattgagtcgttgcagggcagtgcctttcccaactcaaagcgcatctggttgaccggatcgcagcccgatattcgcgtgccgatgcgtgagatccagcttagccccaccgtaacaggcggcagcaaagataacccaacgcttgtcggcaatgaaccggtgccggtctatgatactgccggtccctatggcgacccggaggcgagcattgatgtttatgccgggctggccaaactgcgtgctgactggatcgttaaacgcgatgacacagaacagatcgccgaactgagttccgcctatacccagcaacgcctggccgatgaaggactggatcatctgcgtttcgacaatttgccctgtcctcgccgcgcccgtgccggccgctgtgtgacccagatgcactacgcgcgtcttggggtgatcaccccggaaatggaatttattgccatccgtgaaaatatggggcgcgagcgtattcgcgctgaggtgctgttacaacagcatccgggtaacagctttggtgccagcctgccggaaaatattacccctgaatttgtccgtcaggaagttgccgccgggcgcgccattattcccgccaatattaaccacccggaatccgaaccgatgattattggccgtcatttcctggtgaaaatcaacgccaatatcggcaactcggcggtcagctcctctatcgaagaagaggtggaaaagctggtgtggtcagcgcgctggggcgcagatacggtgatggacctgtccaccgggcgttatatccatgaaacccgcgagtggatcctgcgcaacagcccggttcccattggtactgtgccgatctatcaggcgctggaaaaagtgaacggcgtggcggaagacctcaactgggaaatcttccgcgataccctgctggaacaggctgaacagggcgttgactacttcaccatccatgccggcgtactgctgcgctatgtgccgatgaccgccaaacgtctgaccggcattgtttcgcgcggcggctcgattatggcgaaatggtgcctgtcacatcatcaggagagttttctctatctgcactttcgcgaaatctgcgaaatctgcgccgcttatgacgtttcgctgtcgctcggcgatggcctgcgccccggctcgattcaggacgccaatgatgaggcccagttcgccgagctgcgcacgctgggtgaactgactaaaatcgcctgggaatacgacgtgcaggtgatgattgaagggcctggccatgtgccaatgcagatgatccgcactaatatgacgcaacagctggaacactgccacgaagcgccattctacacgcttggcccgctgaccaccgatatcgctccgggatacgaccatttcacctccgggattggcgccgcaatgattggctggtttggctgcgccatgctgtgctacgtcacacccaaagagcatcttggcttgccgaacaaggaggatgtcaaacagggactgattacctataaaattgcggcccatgccgccgacctggccaaaggccatcccggcgcacagatccgcgacaacgccatgtcaaaagcacgcttcgaattccgttgggaagatcagtttaatctcgccctcgatccgcataccgcccgcgcctaccacgatgaaactctgccgcaggaatcgggcaaggtggcgcacttttgttcgatgtgcggaccgaagttctgttccatgaaaatcactcaggaagtgcgggaatacgccgcacgacaggaagcggaggcaaagcctgtcgagatgggtatggcacagatgtcacgggaattccgcagccggggaggcgagctttatcaccgcgccgatagcccgacgccggaggaaaaagtatga

SEQ ID NO: 28 - DNA, 1716 bp, Erwinia amylovora
  source 1..1716: mol_type = unassigned DNA
atgagcgatgaaaaataccctgggccgctggtggttgaaggcaagctggtcgacgccgaacggctgaagaaacagagcaattatctgcgcggtggcattgccgaagacctgcatgacgggctgaccggcggctttaacggcgataactttctgctgatccgctttcacggcatgtaccagcaggatgaccgcgatattcgcgccgaacgcgccgaacaaaagctggatgcgcgtcatgcgatgatgctgcgctgccgtttgccgggcggcattatgacgccaaagcagtggctggccatcgataagttcgccagcgacaataccctgtacggcagtattcgtttgactaaccgtcaaacgttccagttccacggcatcctgaaaggcaacgtcaaaccggcgcatcagatgctgcacgaagtcgggctggacgcgctggccaccgccaacgatgtcaaccgtaacgtgctgtgcagctcgaacccgattgagtccgaactgcatcaggaagcctacgaatgggcgaaaaagctctccgaacatctgctgccgcgaacccgtgcctatgcggagatctggcttgaccaggaaaaagtcgccaccaccgatgaagagccgatcctcggtgaaacctatctgccgcgtaagttcaaaaccaccgtggtggttccgccgcacaatgacgtagacctgcacgctaacgacatgaactttatcgccatcgctgaacacggcaggctggtcggtttcaacctgctggtcggcggcggtctttctattgaacacggaaataaagccacctatgcccgtaccgccagcgagttcggctatctgccgctgagtaagatactcgacgtggccgaagcagtggtaaccacccagcgtgactggggcaaccgtactgaccgtaaaaatgccaaaaccaagtacacccttgagcgcgtggggccggatgtgtttaaggcagaagtcgagcgccgtgccggtatcacctttgagccgatccgaccgtatgaatttaccacccgtggcgatcgtttcggctgggttaagggcattgacaaccagtggcatctcacgctgtttatcgaaaatggccgtctgctggattatccgggccgcccgctgaaaaccggcctggcagaaattgccaaaatccaccacggtgacttccgcctgaccgcgaatcaaaacctgatcgttgccagggtgccggaggcagaaaaagacaatattcagcggctggcggtcgcgcacggtctgatggaaaacgtcagtcatcagcgtgaaaactcgatggcctgtgtggcattcccgacctgtccgctggcgatggcggaagcagagcgttttctgccgcagttcgtcacccgcgtggaggagatcctcaccgcacacggcgtgggcgatgagcatattgttctgcgcgtcaccggctgccctaacggctgtggtcgtgcgctgctggcggaaatcggcctggtggggaaagcgccggggcgctataacctgcaccttggcggtaaccgcattggtacgcgcatcccgcgcatgtatcgcgaaaatatcaacgaagagcaaattctggcaacccttgatgaactgatcggccgctggtcgcaggagcgcgaagccgatgaaggcttcggtgatttcaccatccgtgccggagtggtgcaaccggtacgcgatccggcccgcgatttctggcaatga

(19) United States
(12) Patent Application Publication
(10) Pub. No.: US 2024/0423211 A1
(43) Pub. Date: Dec. 26, 2024

(54) METHOD AND COMPOSITION FOR REDUCING OR PREVENTING FIRE BLIGHT DISEASE IN A POME FRUIT TREE

(71) Applicant: The Penn State Research Foundation, University Park, PA (US)

(72) Inventors: Timothy W. McNellis, University Park, PA (US); Judith P. Sinn, University Park, PA (US)

(21) Appl. No.: 18/690,364

(22) PCT Filed: Sep. 9, 2022
(86) PCT No.: PCT/US2022/043043

§ 371 Date: Mar. 8, 2024

Related U.S. Application Data
(60) Provisional application No. 63/242,846, filed on Sep. 10, 2021.

(51) Int. Cl. A01N 63/20; A01P 1/00; C12N 1/20

(52) U.S. Cl. CPC A01N 63/20; A01P 1/00; C12N 1/20

(57) ABSTRACT

The present disclosure relates to a method of reducing the occurrence of or preventing fire blight disease in a pome fruit tree. This method involves applying a non-transgenic Erwinia amylovora strain comprising one or more auxotrophic mutations to a pome fruit tree growing in a field under conditions effective to reduce the occurrence of or prevent fire blight disease. The present disclosure also relates to an agricultural composition comprising a non-transgenic Erwinia amylovora strain comprising one or more auxotrophic mutations and an agriculturally acceptable carrier.

This application claims the priority benefit of U.S. Provisional Patent Application Ser. No. 63/242,846, filed Sep. 10, 2021, which is hereby incorporated by reference in its entirety.

This invention was made with government support under Hatch Act Project No. PEN04649 awarded by the United States Department of Agriculture. The government has certain rights in the invention.

### FIELD

The present disclosure relates to methods and compositions for reducing or preventing fire blight disease in a pome fruit tree.

### BACKGROUND

Fire blight disease is a perennial threat to apple and pear production in the United States and remains one of the major causes of apple and pear grower concern. The causal agent, the Gram negative bacterium Erwinia amylovora, enters trees through wounds, flowers, or other natural openings and systemically progresses through the vascular tissue or cambium and parenchyma. The disease was named fire blight because of the burnt appearance of branches and leaves of affected trees. This disease was noted by apple, pear, and quince producers as early as the 1780s.

Apples and pears are valuable crops in the U.S., with gross production values of $3.6 billion and $340 million in 2019, respectively. Apples are also the most popular fruit among U.S. consumers when fresh, frozen, canned, juiced, dried, and otherwise processed fruit are included. Annual costs of fire blight crop loss and management are estimated at about $100 million annually in the U.S. alone. The destructive potential of fire blight can also be illustrated by a successful fire blight eradication effort in Australia in 1997. This effort cost nearly $20 million AUD but averted aggregate economic losses estimated at $870 million AUD over the following two decades.

Fire blight disease manifests mainly as shoot blight and blossom blight. Blossom blight epidemics can be caused by pollinating insects transferring the bacteria from flower to flower. E. amylovora exists on flower surfaces epiphytically for a time before entering the flower tissue. Antibiotic sprays, including streptomycin and copper, can be an effective fire blight preventative when applied to open blooms, although this practice may promote the development of antibiotic resistance in E. amylovora. In fact, streptomycin-resistant E. amylovora isolates have been identified in New York and several other states in the U.S.

In addition to antibiotics and copper, some biopesticide products have been developed for fire blight management. These include products based on Gram-negative bacteria such as Pseudomonas fluorescens strain A506 (BlightBan® A506) and Puntoea vagans strain C9-1 (BlightBan® C9-1), Gram-positive bacteria, including Bacillus subtilis QST317 (Serenade® Optimum) and isolates of the fungus Aureobasidium pullulans (Blossom Protect™).

Currently, growers of pears and apples rely heavily on antibiotic sprays at bloom time to manage fire blight disease. Although a few biologicals and biopesticides are available, these are often not as effective as antibiotics. In addition, antibiotics are no longer permitted in organic fruit production. These limitations highlight the need for novel, organic-compatible fire blight management options.

The present disclosure is directed to overcoming these and other deficiencies in the art.

### SUMMARY

One aspect of the present disclosure relates to a method of reducing the occurrence of or preventing fire blight disease in a pome fruit tree. This method involves applying a non-transgenic Erwinia amylovora strain comprising one or more auxotrophic mutations to a pome fruit tree growing in a field under conditions effective to reduce the occurrence of or prevent fire blight disease.

Another aspect of the present disclosure relates to an agricultural composition comprising a non-transgenic Erwinia amylovora strain comprising one or more auxotrophic mutations and an agriculturally acceptable carrier.

The present disclosure relates to a method for reducing the occurrence of or preventing fire blight disease caused by Erwinia amylovora through the use of auxotrophic strains of E. amylovora. E. amylovora cells multiply epiphytically on host flower stigmatic surfaces before being washed or migrating through water films down into the hypanthium, where invasion into the plant occurs (Thomson, “The Role of the Stigma in Fire Blight Infections,” Phytopathol. 76:476-482 (1986), which is hereby incorporated by reference in its entirety). The epiphytic growth phase of E. amylovora is known to be critical for successful infection and development of fire blight disease (Thomson, “The Role of the Stigma in Fire Blight Infections,” Phytopathol. 76:476-482 (1986), which is hereby incorporated by reference in its entirety). Apple flowers have wet stigmas that produce stigmatic exudates. The stigmatic surface and hypanthium are rich in carbon and nitrogen sources (Campbell et al., “Characterization of Apple Nectar Sugars in Selected Commercial and Crab Apple Cultivars” Fruit Var. J. 44:136-141 (1990); Pusey, “Biochemical Analyses of Pomaceous Stigma Exudates and Relevance to Biological Control of Fire Blight,” Acta Hort. 704:375-377 (2006); Pusey et al., “Characterization of Stigma Exudates in Aqueous Extracts from Apple and Pear Flowers,” Hort Sci. 43:1471-1478 (2008), which are hereby incorporated by reference in their entirety), and E. amylovora presumably uses these carbon and nitrogen sources to fuel multiplication on the flower surface prior to invasion into the plant ((Thomson, “The Role of the Stigma in Fire Blight Infections,” Phytopathol. 76:476-482 (1986); Wilson and Lindow, “Interactions Between the Biological Control Agent Pseudomonas fluorescens A506 and Erwinia amylovora in Pear Blossoms,” Phytopathology 83:117-123 (1993); reviewed in Buban et al., “The Nectary as the Primary Site of Infection by Erwinia amylovora (Burr.) Winslow et al.: A Mini Review,” Plant Syst. Evol. 238:183-194 (2003), which are hereby incorporated by reference in their entirety).

Laboratory tests of biocontrol candidates, such as E. amylovora auxotrophs, are of limited utility and not reliably predictive of outcomes in the field for the control of fire blight disease, because they are performed on detached flowers or small trees growing in the greenhouse rather than the large, mature, fruit-bearing trees in the field. Detached apple flowers only survive for a few days, which is a sufficient amount of time to test bacterial growth, but is insufficient time for the flowers to develop fire blight symptoms. Furthermore, trees blooming in the greenhouse will not develop fire blight when inoculated on the flowers because flowers that are not pollinated will senesce and drop off the plant before fire blight symptoms can develop. In other words, only pollinated host flowers that are attached to a tree will develop fire blight symptoms when inoculated with E. amylovora and can provide a reliable test for efficacy of reducing the occurrence of or preventing fire blight disease.

Another potential reason for the limited utility of laboratory tests of potential biocontrol candidates is that flowers are sprayed with the candidate and then inoculated with virulent E. amylovora by placing a droplet of the virulent bacteria into the hypanthium (floral cup) at the base of the stigmas. This process bypasses the natural infection course, where the virulent bacteria start at the stigma and migrate downwards along the pistil towards the hypanthium over the course of several days while multiplying by several orders of magnitude. Bacterial multiplication on stigmatic surfaces is known to contribute to fire blight infection, while multiplication at the base of the hypanthium is not known to be as important a factor for successful infection.

Moreover, host tissues vary greatly in their susceptibility to infection by auxotrophic E. amylovora strains. For example, apple fruitlets (small immature fruits) are highly susceptible to fire blight disease caused by methionine auxotrophs, but methionine auxotrophs produce almost no fire blight disease when inoculated onto tree shoots on intact trees in the greenhouse (Klee et al., “Erwinia amylovora Auxotrophic Mutant Exometabolomics and Virulence on Apples,” Applied Envir. Micro. 85: e00935-19 (2019), which is hereby incorporated by reference in its entirety). In contrast, tryptophan auxotrophs did not produce disease in apple fruitlets in laboratory tests, but were nearly as virulent as the wild-type E. amylovora on apple shoots (Klee et al., “Erwinia amylovora Auxotrophic Mutant Exometabolomics and Virulence on Apples,” Applied Envir. Micro. 85: e00935-19 (2019), which is hereby incorporated by reference in its entirety).

These results reveal a significant level of uncertainty about the potential behaviors of auxotrophs in different host tissues, including trees under field conditions. Tests in one tissue type do not necessarily predict pathogenicity in other tissues. These differences may be due to the differences in metabolite availability to the bacteria in various host tissues, which are currently largely unknown and must be tested empirically.

As another example, an E. amylovora mutant defective in the virulence regulator gene hrpL was considered non-pathogenic and unable to cause fire blight disease due to lack of production of essential virulence factors (see Johnson et al., “Implications of Pathogenesis by Erwinia amylovora on Rosaceous Stigmas to Biological Control of Fire Blight,” Phytopathology 99:128-138 (2009), which is hereby incorporated by reference in its entirety). Although the hrpL mutant effectively inhibited the growth of virulent E. amylovora on detached apple flowers (see id., FIG. 2E, filled diamonds), the hrpL mutant had only limited effectiveness in inhibiting fire blight in the field (see id., Table 4).

The present disclosure relates to a method and an agricultural composition to reduce occurrence of and prevent fire blight disease by the use of E. amylovora auxotrophs that block or reduce the development of fire blight disease. It was surprisingly shown (and described below) that at least one auxotroph was as effective as antibiotic treatment in preventing fire blight disease in both apple trees and pear trees in the field environment. Apple and pear are related, but different species: Malus domestica and Pyrus communis, respectively. Since the effect was E. amylovora inhibiting itself, it is unlikely to be explained by antibiosis or induction of plant defenses against E. amylovora.

### BRIEF DESCRIPTION OF THE DRAWINGS

FIGS. 1A-C are photographs of fire blight affected ‘Gala’ apple trees. FIG. 1A is a photograph of severe fire blight on a mature apple tree (height˜20 feet). FIG. 1B is a photograph of a young apple tree with severe shoot blight, which can be fatal. FIG. 1C is a photograph of fire blighted leaves and blossoms.

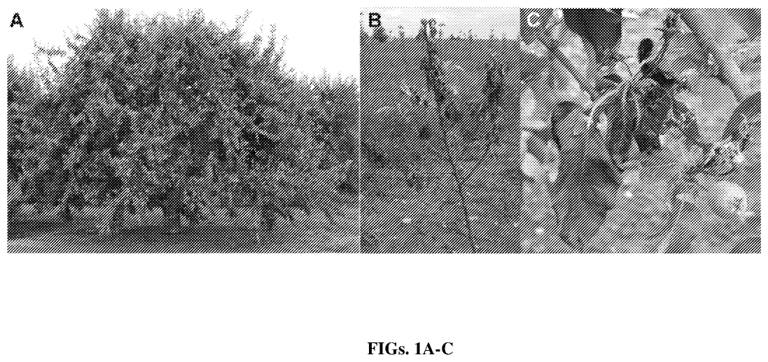

FIGS. 2A-B are charts showing the results of orchard application of a non-pathogenic UV-induced E. amylovora leucine (strain 87-70 “Leucine-1”; FIG. 2A) and arginine (strain HKN06P1 “Arginine-1”; FIG. 2B) auxotrophs to apple trees in separate trials. Five blooming ‘Red Delicious’ (Wenatchee, WA) and ‘Pink Lady’ (Biglerville, PA) apple trees were treated with each product in a randomized block design, followed by inoculation with virulent E. amylovora. FIG. 2A shows the application of the leucine auxotrophic mutant Leucine-1 to ‘Red Delicious’ apple trees at bloom time in Wenatchee, WA. The UV-induced E. amylovora leucine auxotrophic mutant, Leucine-1, reduced fire blight incidence as effectively as antibiotics and a standard organic product. The trees were sprayed at 100% open king blooms. Inoculation was performed the day after product application at 10⁶ Colony Forming Units (“CFU”)/ml wild type E. amylovora. The products were applied at label rates; the E. amylovora leucine auxotroph was applied at 10⁹ CFU/ml in water. Bars sharing the same letter have no statistically significant difference using Fishers t-test (LSD) at p=0.05. FIG. 2B shows the application of the UV-induced Arginine-1 auxotroph to ‘Pink Lady’ apple trees at bloom time using a similar experimental technique to that used in FIG. 2A in Biglerville, PA.

FIG. 3 is a graph showing the population size of pathogenic E. amylovora (strain 153N) on flowers at full bloom, petal fall, and 3 days post petal fall in various treatments of the pear field trial described in Example 5.

FIG. 4 is a graph of the Fire Blight risk at wetness events during the pear trial described in Example 5.

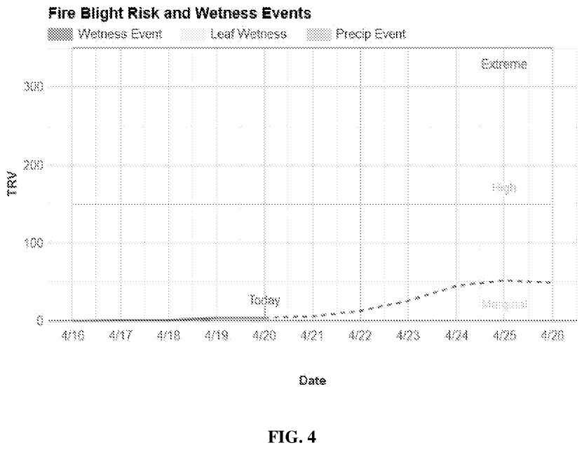

### DETAILED DESCRIPTION

The present disclosure is directed to methods and compositions for reducing or preventing fire blight disease in a pome fruit tree.

Unless otherwise indicated, the definitions and embodiments described in this and other sections are intended to be applicable to all embodiments and aspects of the present disclosure herein described for which they are suitable as would be understood by a person of ordinary skill in the art.

Unless defined otherwise, all technical and scientific terms used in this disclosure have the same meanings as commonly understood by one of ordinary skill in the art to which this disclosure belongs.

Preferences and options for a given aspect, feature, embodiment, or parameter of the invention should, unless the context indicates otherwise, be regarded as having been disclosed in combination with any and all preferences and options for all other aspects, features, embodiments, and parameters of the present disclosure.

In this specification and the appended claims, the singular forms “a,” “an,” and “the” include plural references unless the context clearly dictates otherwise.

The terms “comprising,” “comprises,” and “comprised of” as used herein are synonymous with “including,” “includes,” or “containing,” “contains,” and are inclusive or open-ended and do not exclude additional, non-recited members, elements, or method steps.

The transitional term “comprising,” which is synonymous with “including,” “containing,” or “characterized by,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed subject matter. In some embodiments or claims where the term comprising is used as the transition phrase, such embodiments can also be envisioned with replacement of the term “comprising” with the terms “consisting of” or “consisting essentially of”.

Terms of degree such as “substantially,” “about,” and “approximately” and the symbol “˜” as used herein mean a reasonable amount of deviation of the modified term such that the end result is not significantly changed. These terms of degree should be construed as including a deviation of at least ±0.1% (and up to ±1%, ±5%, or ±10%) of the modified term if this deviation would not negate the meaning of the word it modifies. Unless otherwise clear from context, all numerical values provided herein are modified by the term about. All numerical values provided herein that are modified by terms of degree set forth in this paragraph (e.g., “substantially,” “about,” “approximately,” and “˜”) are also explicitly disclosed without the term of degree. For example, “about 1%” is also explicitly disclosed as “1%”.

The term “and/or” as used herein means that the listed items are present, or used, individually or in combination. In effect, this term means that “at least one of” or “one or more” of the listed items is used or present.

One aspect of the present disclosure relates to a method of reducing the occurrence of or preventing fire blight disease in a pome fruit tree. This method involves applying a non-transgenic Erwinia amylovora strain comprising one or more auxotrophic mutations to a pome fruit tree growing in a field under conditions effective to reduce the occurrence of or prevent fire blight disease.

The term “pome fruit” is used to designate fruits having a fleshy outer layer formed from floral parts surrounding the ovary that expand as the fruit grows and a central core with seeds enclosed in a capsule. Exemplary pome fruits include, but are not limited to, apple, pear, pomegranates, Asian pears, quinces, loquats, crabapples, and the like. In some embodiments, the pome fruit is an apple or a pear. In some embodiments, the pome fruit tree is selected from the group consisting of apple tree, pear tree, pomegranate tree, Asian pear tree, quince tree, loquat tree, and crabapple tree. In some embodiments, the pome fruit tree is an apple tree or a pear tree.

Any suitable apple cultivar may be used in accordance with the methods and compositions of the present disclosure. Exemplary varieties of apple tree varieties that may be used in various embodiments of the present disclosure include, but are not limited to, Adams Pearmain, Alkmene, Akane, Ambrosia, Antonovka, Arlet, Ariane, Arkansas Black, Ashmead's Kernel, Aurora Golden Gala, Baldwin, Ben Davis, Blenheim Orange, Beauty of Bath, Belle de Boskoop, Bohemia, Braeburn, Brina, Cameo, Clivia, Cornish Gilliflower, Cortland, Cox's Orange Pippin, Cripps Pink (Pink Lady), Discovery, Ecolette, Egremont Russet, Elstar, Empire, Esopus Spitzenburg, Fuji, Gala, Ginger Gold, Golden Orange, Golden Delicious, Granny Smith, Gravenstein, Grimes Golden, Haralson, Honeycrisp, Idared, James Grieve, Jazz, Jersey Black, Jonagold, Jonamac, Jonathan, Junaluska, Karmijn de Sonnaville, Knobbed Russet, Liberty, Macoun, McIntosh, Mutsu, Newtown Pippin, Nickajack, Nicola, Nittany, Novaspy, Novamac, Paula Red, Pink Lady, Pink Pearl, Pinova, Rajka, Ralls Genet, Rambo, Red Delicious, Rhode Island Greening, Ribston Pippin, Rome, Rome Beauty, Royal Gala, Roxbury Russet, Rubens (Civni), Santana, Saturn, Sekai Ichi, Spartan, Stayman, Sturmer Pippin, Summerfree, Taliaferro, Topaz, Wealthy, Winter Banana, Worcester Pearmain, Vista Bella, York Imperial or Zestar. Bramley, Calville Blanc d'hiver, Chelmsford Wonder, Flower of Kent, Golden Noble, Norfolk Biffin or Northern Spy Brown Snout, Dabinett, Foxwhelp, Harrison Cider Apple, Kingston Black, Redstreak and Styre.

Any suitable pear cultivar may be used in accordance with the methods and compositions of the present disclosure. Exemplary varieties of pear tree varieties that may be used in various embodiments of the present disclosure include, but are not limited to, Abate Fetel, Alexander Lucas, Asian, Bartlett, Red Bartlett, Bella Lucrative, Beurre Hardy, Bosc, Butirra Precoce Morettini, Taylor's Gold, Catillac, Concorde, Seckel, Red Anjou, Green Anjou, Clairgeau, Clapp's Favourite, Conference, Concorde, Comice, d'Anjou, Easter Beurre, Flemish Beauty, Forelle, Kieffer, Louise Bonne d'Avranches, Passe Crassane, Pound, Seckel, Sheldon, Starkrimson, Williams, Winter Nelis, and P. Barry.

In some embodiments, the pome fruit tree is growing in a field. In some embodiments, the pome fruit tree is growing in a nursery or other location.

Reducing the occurrence of or preventing fire blight disease means that the bacterium that causes fire blight, Erwinia amylovora, is less able or unable to cause fire blight disease compared to untreated trees. Consequently, the incidence or severity of fire blight is reduced or prevented. For example, the incidence of fire blight occurrence or the severity of the disease on a pome fruit tree treated with the non-transgenic Erwinia amylovora strain comprising one or more auxotrophic mutations described herein may be reduced by at least, or at least about, 10%, 20%, 30%, 40%, 50%, 60%, 70%, 80%, 90%, or 100% compared to a pome fruit tree that is left untreated for fire blight, or is treated in another way or by another treatment agent. In some embodiments, the effectiveness of the treatment on disease will be evidenced by a lower number of blackened flowers and flower clusters. In some embodiments, the number of blackened flowers may be reduced by at least, or at least about, 10%, 20%, 30%, 40%, 50%, 60%, 70%, 80%, 90%, or 100% compared to flowers on a pome fruit tree that is left untreated for fire blight, or is treated in another way or by another treatment agent. In some embodiments, the effectiveness of the treatment on disease will be evidenced by a lower number of blighted shoots on trees. In some embodiments, the number of blighted shoots on trees may be reduced by at least, or at least about, 10%, 20%, 30%, 40%, 50%, 60%, 70%, 80%, 90%, or 100% compared to blighted shoots on a pome fruit tree that is left untreated for fire blight, or is treated in another way or by another treatment agent.

An “auxotroph” is a mutant bacterium that requires a particular additional nutrient, which the parent strain does not. An “auxotrophic mutation” is a mutation in a gene or regulatory sequence of a bacterium that causes the bacterium to be an auxotroph for a particular nutrient. For example, a bacterium that has an auxotrophic mutation in a gene such as leuB needed for the production of leucine is a leucine auxotroph and is unable to grow without supplemental leucine.

In some embodiments, the non-transgenic Erwinia amylovora strain comprises one or more auxotrophic mutations. In some embodiments, the one or more auxotrophic mutations is an amino acid auxotrophic mutation. Exemplary gene loci that can be mutated to induce leucine auxotrophy include, without limitation, leuA (SEQ ID NO:1, GenBank Accession No. NC_013961 gene ID 8911958, and WP_013035863.1, which are hereby incorporated by reference in their entirety), leuB (SEQ ID NO:2, 1 GenBank Accession No. NC_013961 gene ID 8913227, and WP_004159730.1, which are hereby incorporated by reference in their entirety), and leuC (SEQ ID NO:3, GenBank Accession No. NC_013961 gene ID 8913228, and WP_004159731.1, which are hereby incorporated by reference in their entirety). The nucleotide sequences for leuA, leuB, and leuC are set forth in Table 5 infra. The sequence of the Erwinia amylovora genome is provided in GenBank Accession No. NC_013961 or GenBank Accession No. NC_013971, which are hereby incorporated by reference in their entirety. In some embodiments, the auxotrophic mutation is a leucine auxotrophic mutation. In some embodiments, the leucine auxotroph is the Leucine-1 auxotroph. In some embodiments, the one or more auxotrophic mutations is in one or more of SEQ ID NOs: 1-3.

In some embodiments, the one or more auxotrophic mutations is an arginine auxotrophic mutation. Exemplary gene loci that can be mutated to induce arginine auxotrophy include, without limitation, argC (SEQ ID NO:4, GenBank Accession No. NC_013961 gene ID 8914116, and WP_004157233.1, which are hereby incorporated by reference in their entirety), argD (SEQ ID NO:5, GenBank Accession No. NC_013961 gene ID 8914716, and

WP_004163439.1, which are hereby incorporated by reference in their entirety), argE (SEQ ID NO: 6 GenBank Accession No. NC_013961 gene ID 8913616, and WP_004154875.1, which are hereby incorporated by reference in their entirety), argG (SEQ ID NO:7 GenBank Accession No. NC_013971 locus tag EAM_RS00700, and WP_004154877.1, which are hereby incorporated by reference in their entirety), argH (SEQ ID NO:8 GenBank Accession No. NC_013961 gene ID 8912440, and WP_004154878.1, which are hereby incorporated by reference in their entirety), and argI (SEQ ID NO:9 GenBank Accession No. NC_013971 gene ID 8050364, and WP_004155151.1, which are hereby incorporated by reference in their entirety). The nucleotide sequences for argC, argD, argE, argG, argH, and argI are set forth in Table 5 infra. In some embodiments, the one or more auxotrophic mutations is an argD auxotrophic mutation. In some embodiments, the arginine auxotroph is the Arginine-1 auxotroph. In some embodiments, the one or more auxotrophic mutations is in one or more of SEQ ID NOs: 4-9.

In some embodiments, the one or more auxotrophic mutations is a proline auxotrophic mutation. Exemplary gene loci that can be mutated to induce proline auxotrophy include, without limitation, proA (SEQ ID NO:10 GenBank Accession No. NC_013971 gene ID 8913654, and WP_004156121.1, which are hereby incorporated by reference in their entirety) and proC (SEQ ID NO:11 GenBank Accession No. NC_013971 gene ID 8912473, and WP_004156171.1, which are hereby incorporated by reference in their entirety). The nucleotide sequences for proA and proC are set forth in Table 5 infra. In some embodiments, the one or more auxotrophic mutations is in one or more of SEQ ID NOs: 10 and 11.

In some embodiments, the one or more auxotrophic mutations is a threonine auxotrophic mutation. Exemplary gene loci that can be mutated to induce threonine auxotrophy include, without limitation, thrA (SEQ ID NO:12 GenBank Accession No. NC_013961 gene ID 8914095, and WP_013035856.1, which are hereby incorporated by reference in their entirety) and thrB (SEQ ID NO:13 GenBank Accession No. NC_013961 gene ID 8914096, and WP_004159786.1, which are hereby incorporated by reference in their entirety). The nucleotide sequences for thrA and thrB are set forth in Table 5 infra. In some embodiments, the one or more auxotrophic mutations is in one or more of SEQ ID NOs: 12 and 13.

In some embodiments, the one or more auxotrophic mutations is a tryptophan auxotrophic mutation. Exemplary gene loci that can be mutated to induce tryptophan auxotrophy include, without limitation, trpB (SEQ ID NO: 14 GenBank Accession No. NC_013961 gene ID 8914096, and WP_004157750.1, which are hereby incorporated by reference in their entirety), trpCF (SEQ ID NO:15, GenBank Accession No. NC_013961 gene ID 8914125, and WP_004157749.1, which are hereby incorporated by reference in their entirety), trpE (SEQ ID NO: 16, GenBank Accession No. NC_013971 locus tag EAM_RS09045, and WP_004157746.1, which are hereby incorporated by reference in their entirety), and trpG (SEQ ID NO:17, GenBank Accession No. NC_013961 gene ID 8911683, and WP_004157746.1, which are hereby incorporated by reference in their entirety). The nucleotide sequences for trpB, trpCF, trpE, and trpG are set forth in Table 5 infra. In some embodiments, the one or more auxotrophic mutations is in one or more of SEQ ID NOs: 14-17.

In some embodiments, the one or more auxotrophic mutations is a nucleotide auxotrophic mutation. In some embodiments, the one or more auxotrophic mutations is a purine auxotrophic mutation. In some embodiments, the one or more auxotrophic mutations is a pyrimidine auxotrophic mutation. In some embodiments, the one or more auxotrophic mutations is a guanine auxotrophic mutation. Exemplary gene loci that can be mutated to induce purine, pyrimidine, or guanine auxotrophy include, without limitation, purC (SEQ ID NO:18 GenBank Accession No. NC_013961 gene ID 8914894, and WP_004159011.1, which are hereby incorporated by reference in their entirety), pyrB (SEQ ID NO:19 GenBank Accession No. NC_013961 gene ID 8913728, and WP_004155150.1, which are hereby incorporated by reference in their entirety), and guaB (SEQ ID NO:20 GenBank Accession No. NC_013961 gene ID 8913023, and WP_004159092.1, which are hereby incorporated by reference in their entirety). The nucleotide sequences for purC, pyrB, and guaB are set forth in Table 5 infra. In some embodiments, the one or more auxotrophic mutations is in one or more of SEQ ID NOs: 18-20.

In some embodiments, the one or more auxotrophic mutations is a sugar utilization auxotrophic mutation. Exemplary gene loci that can be mutated to induce sugar utilization auxotrophy include, without limitation, gapA (SEQ ID NO:21 GenBank Accession No. NC_013961 gene ID 8914474, and WP_004157815.1, which are hereby incorporated by reference in their entirety) and pts/(SEQ ID NO:22 GenBank Accession No. NC_013961 gene ID 8913691, and WP_004158917.1, which are hereby incorporated by reference in their entirety). The ptsI gene is predicted to encode Enzyme I of the phosphoenolpyruvate dependent phosphotransferase system. The gapA gene encodes Glyceraldehyde phosphate dehydrogenase (“GAPDH”). In some embodiments, the one or more auxotrophic mutations is a sugar utilization gapA auxotrophic mutation. In some embodiments, the one or more auxotrophic mutations is a sugar utilization pts/auxotrophic mutation. The nucleotide sequences for gapA and ptsI are set forth in Table 5 infra. In some embodiments, the one or more auxotrophic mutations is in one or more of SEQ ID NOs: 21 and 22.

In certain embodiments, the one or more auxotrophic mutations is a nitrogen utilization auxotrophic mutation. Exemplary gene loci that can be mutated to induce nitrogen auxotrophy include, without limitation, glnA (SEQ ID NO:23, GenBank Accession No. NC_013961 gene ID 8912964, and WP_004154751.1, which are hereby incorporated by reference in their entirety) and gltB (SEQ ID NO:24 GenBank Accession No. NC_013961 gene ID 8912643, and WP_004155074.1, which are hereby incorporated by reference in their entirety). The gltB gene encodes a subunit of glutamine 2-oxoglutarate amidotransferase (“GOGAT”), which plays a central role in high affinity ammonium assimilation, especially in high-carbon environments, the utilization of organic nitrogen sources, and glutamic acid biosynthesis (van Heeswijk et al., “Nitrogen Assimilation in Escherichia coli: Putting Molecular Data into a Systems Perspective,” Microbiol. Mol. Biol. Rev. 77:628-95 (2013), which is hereby incorporated by reference in its entirety). The glnA gene encodes glutamine synthetase, which is expected to be defective in lower affinity ammonium uptake. In some embodiments, the one or more auxotrophic mutations is a nitrogen utilization auxotrophic glnA mutation. In some embodiments, the one or more auxotrophic mutations is a nitrogen utilization auxotrophic gltB mutation. The nucleotide sequences for glnA and gltB are set forth in Table 5 infra. In some embodiments, the one or more auxotrophic mutations is in one or more of SEQ ID NOs: 23 and 24.

In some embodiments, the one or more auxotrophic mutations is a high affinity siderophore-based iron uptake auxotrophic mutation. An exemplary gene locus that can be mutated to induce high affinity siderophore-based iron uptake auxotrophy includes, without limitation, foxR (SEQ ID NO:25, GenBank Accession No. AJ223062.1, which is hereby incorporated by reference in its entirety). The nucleotide sequence for foxR is set forth in Table 5 infra. In some embodiments, the one or more auxotrophic mutations is in SEQ ID NO:25.

In some embodiments, the one or more auxotrophic mutations is a glucosamine auxotrophic mutation. An exemplary gene locus that can be mutated to induce glucosamine auxotrophy includes, without limitation, glmS (SEQ ID NO:26 GenBank Accession No. NC_013961 gene ID 8912244, and WP_004161228.1, which are hereby incorporated by reference in their entirety). The nucleotide sequence for glmS is set forth in Table 5 infra. In some embodiments, the one or more auxotrophic mutations is in SEQ ID NO: 26.

In some embodiments, the one or more auxotrophic mutations is a vitamin auxotrophic mutation. An exemplary gene locus that can be mutated to induce vitamin auxotrophy includes, without limitation, thiC (SEQ ID NO:27 GenBank Accession No. NC_013961 gene ID 8914086, and WP_004155007.1, which are hereby incorporated by reference in their entirety). In some embodiments, the one or more auxotrophic mutation is a thiamine auxotrophic mutation. The nucleotide sequence for thiC is set forth in Table 5 infra. In some embodiments, the one or more auxotrophic mutations is in SEQ ID NO: 27.

In some embodiments, the one or more auxotrophic mutations is an inorganic sulfur uptake auxotrophic mutation. An exemplary gene locus that can be mutated to induce inorganic sulfur uptake auxotrophy includes, without limitation, cysI (SEQ ID NO:28 GenBank Accession No. NC_013961 gene ID 8912645, and WP_004155804.1, which are hereby incorporated by reference in their entirety). The nucleotide sequence for cysI is set forth in Table 5 infra. In some embodiments, the one or more auxotrophic mutations is in SEQ ID NO: 28.

In some embodiments, the one or more auxotrophic mutations are selected from the group consisting of a leucine auxotrophic mutation, an arginine auxotrophic mutation, a proline auxotrophic mutation, a threonine auxotrophic mutation, a tryptophan auxotrophic mutation, a guanine auxotrophic mutation; a purine auxotrophic mutation, a pyrimidine auxotrophic mutation, a sugar utilization gapA auxotrophic mutation, a sugar utilization ptsI auxotrophic mutation, a nitrogen utilization glnA auxotrophic mutation, a nitrogen utilization gltB auxotrophic mutation, a high affinity siderophore-based iron uptake auxotrophic mutation, a glucosamine auxotrophic mutation, a thiamine auxotrophic mutation, an inorganic sulfur uptake auxotrophic mutation, and combinations thereof. In some embodiments, the one or more auxotrophic mutations are selected from the group consisting of a leucine auxotrophic mutation and an arginine auxotrophic mutation.

In some embodiments, the one or more auxotrophic mutations are higher-order mutants combining arginine or leucine auxotrophy with additional defects involving traits including, for example, one or more of nucleotide biosynthesis, sugar and/or nitrogen utilization, and iron or sulfur utilization.

In some embodiments, the one or more auxotrophic mutations comprises an arginine auxotrophic mutation and a sugar utilization gapA auxotrophic mutation. In some embodiments, the one or more auxotrophic mutations comprises an arginine auxotrophic mutation and a nitrogen utilization gltB auxotrophic mutation. In some embodiments, the one or more auxotrophic mutations comprise an arginine auxotrophic mutation and a nitrogen utilization glnA auxotrophic mutation. In some embodiments, the one or more auxotrophic mutations comprise an arginine auxotrophic mutation, a nitrogen utilization glnA auxotrophic mutation, and a nitrogen utilization gltB auxotrophic mutation. In some embodiments, the one or more auxotrophic mutations comprise an arginine auxotrophic mutation, a sugar utilization gapA auxotrophic mutation, and a nitrogen utilization gltB auxotrophic mutation. In some embodiments, the one or more auxotrophic mutations comprise an arginine auxotrophic mutation, a sugar utilization ptsI auxotrophic mutation, and a nitrogen utilization gltB auxotrophic mutation. In some embodiments, the one or more auxotrophic mutations comprise an arginine auxotrophic mutation and a high affinity siderophore-based iron uptake auxotrophic mutation. In some embodiments, the one or more auxotrophic mutations comprise an arginine auxotrophic mutation, a guanine auxotrophic mutation, a leucine auxotrophic mutation, a thiamine auxotrophic mutation, and a threonine auxotrophic mutation. In some embodiments, the one or more auxotrophic mutations comprise an arginine auxotrophic mutation and a sulfur uptake auxotrophic mutation.

In some embodiments, the one or more auxotrophic mutations is selected from the group consisting of a leucine and an arginine auxotrophic mutation; an arginine auxotrophic mutation and a sugar utilization gapA auxotrophic mutation; an arginine auxotrophic mutation and a nitrogen utilization gltB auxotrophic mutation; an arginine auxotrophic mutation and a nitrogen utilization glnA auxotrophic mutation; an arginine auxotrophic mutation, a nitrogen utilization glnA auxotrophic mutation, and a nitrogen utilization gltB auxotrophic mutation; an arginine auxotrophic mutation, a sugar utilization gapA auxotrophic mutation, and a nitrogen utilization gltB auxotrophic mutation; an arginine auxotrophic mutation, a sugar utilization ptsI auxotrophic mutation, and a nitrogen utilization gltB auxotrophic mutation; an arginine auxotrophic mutation and a high affinity siderophore-based iron uptake auxotrophic mutation; an arginine auxotrophic mutation and a guanine auxotrophic mutation, a leucine auxotrophic mutation, a thiamine auxotrophic mutation, and a threonine auxotrophic mutation; and an arginine auxotrophic mutation and a sulfur uptake auxotrophic mutation.

Mutagenesis can be used to induce mutations in Erwinia amylovora genes to cause auxotrophy. In some embodiments, a non-transgenic auxotrophic mutation may be induced by treatment with a mutagenic agent. Any suitable mutagenic agent can be used for embodiments of the present disclosure. For example, mutagens creating point mutations, deletions, insertions, rearrangements, transversions, transitions, or any combination thereof may be used. Suitable radiation mutagens include, without limitation, ultraviolet (“UV”) light, x-rays, gamma rays, and fast neutrons. Suitable chemical mutagens include, but are not limited to, ethyl methanesulfonate (EMS), methylmethane sulfonate (MMS), N-ethyl-N-nitrosourea (ENU), triethylmelamine (TEM), N-methyl-N-nitrosourea (MNU), procarbazine, chlorambucil, cyclophosphamide, diethyl sulfate, acrylamide monomer, melphalan, nitrogen mustard, vincristine, dimethylnitrosamine, N-methyl-N′-nitro-nitrosoguanidine 25 (MNNG), nitrosoguanidine, 2-aminopurine, 7, 12 dimethyl-benz(a)anthracene (DMBA), ethylene oxide, hexamethylphosphoramide, bisulfan, diepoxyalkanes (diepoxyoctane (DEO), diepoxybutane (DEB), 2-methoxy-6-chloro-9[3-(ethyl-2-chloro-ethyl) aminopropylamino] acridine dihydrochloride (ICR-170), sodium azide, formaldehyde, or combinations thereof. In some embodiments, the non-transgenic mutagenic agent is UV light.

In some embodiments, a mutation may be induced by genome editing. Genome editing is a type of genetic engineering in which DNA is inserted, replaced, or removed, or any combination thereof, from a genome using artificially engineered nucleases, or “molecular scissors.” The nucleases typically create double-stranded breaks (DSBs) at desired locations in the genome, and harness the cell's endogenous mechanisms to repair the induced break by processes of homology dependent repair (HDR) or non-homologous end-joining (NHEJ). Any method of genome editing may be used in the embodiments of the present disclosure.

CRISPR/Cas type RNA-guided endonucleases provide an efficient system for inducing genetic modifications in genomes of many organisms. Non-limiting examples of genome editing nucleases include Cas1, Cas1B, Cas2, Cas3, Cas4, Cas5, Cas6, Cas7, Cas8, Cas9 (also known as Csn1 and Csx12), Cas10, Cas12a (Cpf1), Csy1, Csy2, Csy3, Cse1, Cse2, Cse1, Cse2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, Mad7, or homologs, modified versions, and endonuclease inactive versions thereof. An example of a fusion protein to Cas9 is a cytidine deaminase-Cas9 fusion protein used in cytidine base editing to mutate nucleotides in target genes without generating double-strand breaks as described in Komor et al., “Programmable Editing of a Target Base in Genomic DNA without Double-Stranded DNA Cleavage,” Nature 533:420-424 (2016), which is hereby incorporated by reference in its entirety. The use of CRISPR guide RNA in conjunction with CRISPR/Cas technology to target RNA is also described in Wiedenheft et al., “RNA-Guided Genetic Silencing Systems in Bacteria and Archaea,” Nature 482:331-338 (2012); Zhang et al., “Multiplex Genome Engineering Using CRISPR/Cas Systems,” Science 339:819-23 (2013); and Gaj et al., “ZFN, TALEN, and CRISPR/Cas-based Methods for Genome Engineering,” Cell 31:397-405 (2013), each of which is hereby incorporated by reference in their entirety.

There are typically two distinct components to a CRISPR system, a guide RNA (“gRNA”) and a genome editing endonuclease. The gRNA uses a CRISPR RNA (“crRNA”) comprising a DNA targeting segment that can be engineered to contain a complementary stretch of nucleotide sequence (e.g., at least 10 nucleotides) to target a DNA site for binding and subsequent modification by CRISPR genome editing nuclease. The length of a crRNA may range from about 15 nucleotides to about 60 nucleotides. The crRNA can be chemically synthesized and can also be engineered to include a ribonucleotide analog or a modified form thereof, or an analog of a modified form, or non-natural nucleosides.

Depending on the genome editing nuclease used, the gRNA can also comprise a trans-activating crRNA (“tracrRNA”). Such is the case with Cas9, for example. The tracrRNA is a small RNA sequence that forms a binding handle used by the CRISPR protein. The tracrRNA can be chemically synthesized and can also be engineered to include a ribonucleotide analog or a modified form thereof, or an analog of a modified form, or non-natural nucleosides.

When the gRNA and the gene editing endonuclease are introduced into the cell, the genomic target sequence can be modified or permanently disrupted to create a loss-of-function mutation(s). A complex of a genome editing nuclease with a gRNA is called a ribonucleotide particle or ribonucleoprotein (RNP) complex. The RNP complex is recruited to the target sequence by the base-pairing between the gRNA sequence, which has a region of complementarity to the target sequence in the genomic DNA.

Other genome editing technologies can also be used in the methods and compositions of the present disclosure. Some examples include Zinc Finger Nucleases (“ZFNs”), which are artificial restriction enzymes generated by fusing a zinc finger DNA-binding domain to a DNA-cleavage domain; TALENs, which are sequence-specific endonucleases that includes a transcription activator-like effector (“TALE”) and a FokI endonuclease, and Meganucleases, without limitation.

In some embodiments, mutations may be induced using targeted deletion using homologous recombination (see Klee et al., “The Apple Fruitlet Model System for Fire Blight Disease,” Methods in Mol. Biol. 1991:187-198 (2019), which is hereby incorporated by reference in its entirety).

A mutation may be detected through any method known to those of skill in the art. In some embodiments, a mutation is detected by sequencing.

In some embodiments, the one or more auxotrophic mutations is a UV-induced mutation. In some embodiments, the one or more auxotrophic mutations is a deletion-induced mutation. In some embodiments, the one or more auxotrophic mutations is a genome edited-mutation. In other embodiments, the one or more auxotrophic mutations may be induced through UV mutagenesis, deletion mutagenesis, or genome editing.

In some embodiments, the method further comprises applying the non-transgenic Erwinia amylovora strain comprising one or more auxotrophic mutations to a pome tree at flowering. In some embodiments, application is made during flower bloom, since the flower (blossom) is the primary infection site.

In some embodiments, applying is carried out by application of a composition comprising the non-transgenic Erwinia amylovora strain to pome fruit trees during a period from their flowering stage in which the first flower blooms to a stage in which most flowers wither and drop from the trees. However, to obtain favorable results, it may be convenient to apply the non-transgenic Erwinia amylovora strain during a period from mid (40-50%) or late bloom (90-100%). In some embodiments, the pome fruit tree has at least, or at least about, 10%, 20%, 30%, 40%, 50%, 60%, 70%, 80%, 90%, 95%, or 100% of flowers in bloom. In some embodiments, the pome fruit tree has at least 50% of flowers in bloom. In some embodiments, applying the non-transgenic Erwinia amylovora strain may be carried out immediately after full bloom of terminal flowers to two days thereafter. By the term “full bloom of terminal flowers,” it is meant a timing when 80% of total terminal flowers on the pome fruit tree concerned are in bloom.

In some embodiments, the non-transgenic Erwinia amylovora strain comprising one or more auxotrophic mutations is applied in an effective amount to reduce the occurrence of or prevent fire blight disease caused by Erwinia amylovora. An effective amount means that quantity of Erwinia amylovora auxotrophic cells sufficient to inhibit the incidence or development of pathogenic Erwinia amylovora when applied to pome fruit blossoms. In some embodiments, an effective amount comprises a concentration range from about 1×10⁶ to 1×10¹⁰ colony forming units (“CFU”)/ml of the non-transgenic Erwinia amylovora strain, or any amount or range therein. In some embodiments, an effective amount is about 10⁶-10⁹ CFU/ml of the non-transgenic Erwinia amylovora strain, or any amount or range therein. In some embodiments an effective amount is about 10⁶, 10⁷, 10⁸, 10⁹, or 10¹⁰ CFU/ml of the non-transgenic Erwinia amylovora strain. The effective amount can be determined by environmental factors, which can affect the amount of auxotrophic strain to be used. Such environmental factors may include, for example, wind, humidity, temperature, time of day, time of year, etc.

In some embodiments, the non-transgenic Erwinia amylovora strain comprising one or more auxotrophic mutations is applied to a pome fruit tree when ambient temperatures are above a minimum temperature of 33° F., 34° F., 35° F., 36° F., 37° F., 38° F., 39° F., 40° F., 41° F., 42° F., 43° F., 44° F., 45° F., 46° F., 47° F., 48° F., 49° F., 50° F., or greater than 50° F. In some embodiments, the non-transgenic Erwinia amylovora strain comprising one or more auxotrophic mutations is applied to a pome fruit tree when the average coldest temperature of the day or the actual coldest temperature of the day is above 33° F., 34° F., 35° F., 36° F., 37° F., 38° F., 39° F., 40° F., 41° F., 42° F., 43° F., 44° F., 45° F., 46° F., 47° F., 48° F., 49° F., 50° F., or greater than 50° F. In some embodiments, the non-transgenic Erwinia amylovora strain comprising one or more auxotrophic mutations is applied to a pome fruit tree when ambient temperatures are above a minimum temperature of about 33-38° F., 35-40° F., 37-42° F., 39-44° F., 41-46° F., 43-48° F., or 45-50° F. In some embodiments, the non-transgenic Erwinia amylovora strain comprising one or more auxotrophic mutations is applied to a pome fruit tree when the average ambient temperature or actual ambient temperature is above a minimum temperature of about 33-38° F., 35-40° F., 37-42° F., 39-44° F., 41-46° F., 43-48° F., or 45-50° F.

In some embodiments, the non-transgenic Erwinia amylovora strain comprising one or more auxotrophic mutations is incorporated into compositions suitable for application to pome fruit trees, for example for blossom application. The non-transgenic Erwinia amylovora strain can be mixed with any agriculturally acceptable carrier or suitable agronomically acceptable carrier which does not interfere with the activity of the auxotroph.

In some embodiments, applying the non-transgenic Erwinia amylovora strain to a pome fruit tree is carried out with a composition comprising the non-transgenic Erwinia amylovora strain comprising one or more auxotrophic mutations and an agriculturally acceptable carrier. In non-limiting examples, the agronomically acceptable carrier is water or buffer. In some embodiments, the agriculturally acceptable carrier comprises water. Where the non-transgenic Erwinia amylovora strain is applied as a suspension or emulsion in a liquid carrier, the suspension or emulsion may optionally contain conventional additives such as surfactants, wetting agents, or chemical inhibitors as known in the art.

In some embodiments, the agriculturally acceptable carrier is a solid. Suitable solid carriers include, without limitation, crude protein, dry milk products, mineral earths such as silicates, silica gels, talc, kaolins, limestone, lime, chalk, bole, loess, clays, dolomite, diatomaceous earth, calcium sulfate, magnesium sulfate, magnesium oxide, ground synthetic materials, fertilizers (e.g., ammonium sulfate, ammonium phosphate, ammonium nitrate, ureas, and products of vegetable origin, such as cereal meal, tree bark meal, wood meal and nutshell meal); cellulose powders; and other solid carriers. In some embodiments, the agriculturally acceptable carrier comprises a milk product.

In some embodiments, the composition further comprises a surfactant. Suitable surfactants (also known as adjuvants, wetters, tackifiers, dispersants, or emulsifiers) include, without limitation, alkali metal, alkaline earth metal, and ammonium salts of aromatic sulfonic acids (e.g., ligninsulfonic acid (Borresperse® types, Borregard, Norway)) phenolsulfonic acid, naphthalenesulfonic acid (Morwet® types, Akzo Nobel, U.S.A.), dibutylnaphthalene-sulfonic acid (Nekal® types, BASF, Germany), fatty acids, alkylsulfonates, alkylarylsulfonates, alkyl sulfates, laurylether sulfates, fatty alcohol sulfates, sulfated hexa-, hepta- and octadecanolates, sulfated fatty alcohol glycol ethers, condensates of naphthalene or of naphthalenesulfonic acid with phenol and formaldehyde, polyoxy-ethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquid and proteins, denatured proteins, polysaccharides (e.g., methylcellulose), hydrophobically modified starches, polyvinyl alcohols (Mowiol® types, Clariant, Switzerland), polycarboxylates (Sokolan® types, BASF, Germany), polyalkoxylates, polyvinylamines (Lupasol® types, BASF, Germany), polyvinylpyrrolidone and copolymers thereof, Tween®80, and NU-FILM P (a mixture with a polyalkyloxy compound, Miller, PA).

In some embodiments, the composition can include a thickener. Non-limiting examples of thickeners (i.e., compounds that impart a modified flowability to formulations (i.e., high viscosity under static conditions and low viscosity during agitation)) are polysaccharides and organic and inorganic clays such as Xanthan gum (Kelzan®, CP Kelco, U.S.A.), Rhodopol® 23 (Rhodia, France), Veegum® (R.T. Vanderbilt, U.S.A.) or Attaclay® (Engelhard Corp., NJ, USA).

In some embodiments, the composition can include an anti-freeze or osmotic protectant. Non-limiting examples of suitable anti-freezing agents are ethylene glycol, propylene glycol, urea, glycerol, and glycerin. In some embodiments, the agriculturally acceptable carrier comprises glycerol.

In some embodiments, the composition can include an antifoaming agent. Non-limiting examples of anti-foaming agents are silicone emulsions (e.g., Silikon® SRE, Wacker, Germany and Rhodorsil®, Rhodia, France), long chain alcohols, fatty acids, salts of fatty acids, fluoroorganic compounds, and mixtures thereof.

In some embodiments, the composition can include a binder and/or tackifier. Non-limiting examples of tackifiers or binders include polyvinylpyrrolidones, polyvinylacetates, polyvinyl alcohols, and cellulose ethers (e.g., Tylose®, Shin-Etsu, Japan).

In some embodiments, the composition can include a powder. Powders, materials for spreading, and dusts can be prepared by mixing or concomitantly grinding the active compounds with at least one solid carrier.

In some embodiments, the composition further comprises one or more compounds for which the Erwinia amylovora strain is auxotrophic. For example, if the Erwinia amylovora strain is a leucine auxotroph, the one or more compounds included in the composition may be leucine. If the Erwinia amylovora strain is auxotrophic for more than one compound, the one or more compounds included in the composition may be one or multiple compounds. In some embodiments, the one or more compounds included in the composition (and for which the Erwinia amylovora strain is auxotrophic) is leucine and/or arginine. In some embodiments, 1-100 mM leucine is included in the composition. In some embodiments, about 1-10 mM leucine, about 10-50 mM leucine, about 50-100 mM leucine, or about 100-1000 mM leucine is included in the composition. In some embodiments, more than 1M leucine is included in the composition. In some embodiments, 1-100 mM arginine is included in the composition. In some embodiments, about 1-10 mM arginine, about 10-50 mM arginine, about 50-100 mM arginine, or about 100-1000 mM arginine is included in the composition. In some embodiments, more than 1M arginine is included in the composition.

In some embodiments, the composition is formulated for dilution with water. Thus, the composition may be formulated as a water soluble concentrate, a dispersable concentrate, an emulsifiable concentrate, an emulsion, a suspension, water-dispersible granules and/or water-soluble granules, water-dispersible powders and/or water-soluble powders, or a gel.

In the case of water-soluble concentrates, the formulation may include e.g., about 10 parts by weight of the composition of the present disclosure dissolved in about 90 parts by weight of water or a water-soluble solvent. As an alternative, wetting agents or other auxiliaries may be added. Other proportions are also contemplated. For example the formulation of the composition may include about 15, 20, 25, 30, 35, 40, 45, 50, 55, 60, 65, 70, 75, 80, or 85 parts by weight of the composition of the present disclosure dissolved in about 85, 80, 75, 70, 65, 60, 55, 50, 45, 40, 35, 30, 25, 20, or 15 parts by weight of water or a water-soluble solvent.

In some embodiments, the non-transgenic Erwinia amylovora auxotrophic strain is present in the composition as a wet paste or in a dry form such as a lyophilized form.

The non-transgenic Erwinia amylovora strain can also be formulated to include other fire blight biocontrol strains. In some embodiments, the agricultural composition comprises two Erwinia amylovora auxotrophic strains. In some embodiments, the agricultural composition comprises more than two Erwinia amylovora auxotrophic strains.

Methods of applying an agricultural composition to growing plants (including trees in a field) are well known in the art and include, but are not limited to, spraying, wetting, dipping, misting, drenching, showering, fogging, soaking, dampening, drizzling, dousing, and splashing. In some embodiments of carrying out methods disclosed herein, said applying is carried out by spraying.

In some embodiments, said applying reduces fire blight disease by at least 10% compared to an untreated pome fruit tree. In some embodiments, said applying reduces fire blight disease by at least 20% compared to an untreated pome fruit tree. In some embodiments, said applying reduces fire blight disease by at least 30% compared to an untreated pome fruit tree. In some embodiments, said applying reduces fire blight disease by at least 40%, 50%, 60%, 70%, 80%, 90%, 95%, or 100% on fruit of the treated fruit tree compared to pome fruits of an untreated pome fruit tree or a pome fruit tree treated for fire blight disease with a treatment other than the non-transgenic Erwinia amylovora strain comprising one or more auxotrophic mutations. In some embodiments, said applying reduces occurrence of or prevents fire blight disease of blossoms of a pome fruit tree compared to blossoms of an untreated pome fruit tree or a pome fruit tree not treated with non-transgenic Erwinia amylovora strain comprising one or more auxotrophic mutations. In some embodiments, said applying reduces occurrence of or prevents fire blight disease of blossoms of a pome fruit tree by at least 10%, 20%, 30%, 40%, 50%, 60%, 70%, 80%, 90%, 95%, or 100% compared to blossoms of an untreated pome fruit tree or a pome fruit tree not treated with non-transgenic Erwinia amylovora strain comprising one or more auxotrophic mutations.

The appearance of most fruit varieties is a clear, smooth surface. Fruit with imperfections or blemishes on the fruit surfaces known as russetting are generally considered inferior by consumers. Cracks in the cuticle layer of the fruit surface and/or the death of individual epidermal cells lead to the rough, browned appearance that is known as russetting. Russetting can be caused by disease control agents, such as organic compatible agents, which can limit their utility due to fruit quality decline. In some embodiments, the methods and compositions of the present disclosure reduce the occurrence of russetting on pome fruits of the treated pome fruit tree compared to pome fruits of an untreated pome fruit tree or a pome fruit tree treated for fire blight disease with an organic compatible treatment other than the non-transgenic Erwinia amylovora strain comprising one or more auxotrophic mutations. In some embodiments, russetting is reduced by 10%, 20%, 30%, 40%, 50%, 60%, 70%, 80%, 90%, 95%, or 100% compared to an untreated pome fruit tree or a pome fruit tree treated for fire blight disease with an organic compatible treatment other than the non-transgenic Erwinia amylovora strain of the present disclosure. In some embodiments, russetting is reduced by 10%, 20%, 30%, 40%, 50%, 60%, 70%, 80%, 90%, 95%, or 100% when treated with the non-transgenic Erwinia amylovora auxotrophic strain of the present disclosure compared to an untreated pome fruit tree.

In some embodiments, the field is located in an environment susceptible to fire blight disease mediated by Erwinia amylovora.

This aspect of the present disclosure can be carried out with any of the embodiments disclosed herein.

Another aspect of the present disclosure relates to an agricultural composition comprising a non-transgenic Erwinia amylovora strain comprising one or more auxotrophic mutations and an agriculturally acceptable carrier.

This aspect of the present disclosure can be carried out with any of the embodiments disclosed herein.

Suitable auxotrophic mutations for use in the compositions and methods of the present disclosure are described in more detail supra. In some embodiments of the agricultural composition described herein, the one or more auxotrophic mutation is a leucine auxotrophic mutation. In some embodiments, the leucine auxotroph is the Leucine-1 auxotroph. In some embodiments, the one or more auxotrophic mutations is an arginine auxotrophic mutation. In some embodiments, the arginine auxotroph is the Arginine-1 auxotroph.

In some embodiments, the agricultural composition is a composition that can be certified as organic (e.g., it would meet the standards required by at least one certifying body, such as Organic Materials Review Institute (OMRI) or any other similar certifying body). An organic composition does not include antibiotics, synthetic pesticides and fertilizers, genetically engineered (transgenic) organisms, synthetic growth hormones, artificial flavors, colors, preservatives, among other requirements.

A person of ordinary skill in the art would readily understand that the methods and compositions described herein may be modified and optimized for particular embodiments of choice. The following examples are intended to illustrate but not limit the invention.

### EXAMPLES

### Example 1—Isolation of Non-Transgenic Auxotrophic Strains of Erwinia amylovora

Non-transgenic auxotrophic mutations were induced using ultraviolet (UV) light mutagenesis in Erwinia amylovora strains. UV mutagenesis was performed on two different isolates of wild-type E. amylovora following a classic auxotrophic mutant selection procedure (Davis, “The Isolation of Biochemically Deficient Mutants of Bacteria by Means of Penicillin,” Proc. Natl. Acad. Sci. USA 35:1-10 (1949), which is hereby incorporated by reference in its entirety). In brief, bacterial suspensions were exposed to UV light in a microbiological safety cabinet for a time sufficient to result in 90-99% mortality (˜5 minutes). The bacteria were then grown in M9 minimal medium with 100 μg/ml carbenicillin for 24 hours to select against prototrophs, and then plated on LB to allow potential auxotrophs to grow. Single colonies from the LB plates were grown in 96 well plates and then replica plated using a 96 prong replicator on LB and M9 plates to identify auxotrophs, which grew on LB, but not on M9. The selection procedure was quite effective and resulted in a population of about 30% auxotrophic mutants. The auxotrophs were replica plated onto M9 medium plates individually supplemented with metabolites of interest, including arginine, leucine, and lysine.

Through this process, a Washington state Erwinia amylovora strain 87-70 with a UV-induced, non-transgenic leucine auxotrophic mutation was identified and called “Leucine-1”. A Pennsylvania state Erwinia amylovora strain HKN06P1 with a UV-induced non-transgenic arginine auxotrophic mutation was also identified, called “Arginine-1”.

### Prophetic Example 2—Further Isolation of Non-Transgenic Auxotrophic Strains of Erwinia amylovora

Genomes of auxotrophs of interest will be sequenced and the lesion(s) leading to auxotrophy will be identified by comparison to the CFBP 1430 reference genome sequence, as previously described (see Klee et al., “Extragenic Suppression of Elongation Factor P Gene Mutant Phenotypes in Erwinia amylovora,” J. Bacteriol. 201: e00722-18 (2019), which is hereby incorporated by reference in its entirety). Higher order auxotrophic mutants will be generated by additional mutagenesis of the auxotroph of interest and selection steps for the second desired auxotrophic trait. In those selections, M9 medium would be supplemented with the metabolite needed due to the first mutation.

Additional non-transgenic auxotrophic lines are generated by targeted deletion using homologous recombination methods as disclosed in Klee et al., “Extragenic Suppression of Elongation Factor P Gene Mutant Phenotypes in Erwinia amylovora,” J. Bacteriol. 201: e00722-18 (2019) and Klee et al, “Virulence Genetics of an Erwinia amylovora Putative Polysaccharide Transporter Family Member,” J. Bacteriol. 202: e00390-20 (2020), each of which is hereby incorporated by reference in its entirety. Deletions are clean deletions that do not include an antibiotic cassette, so multiple auxotrophic mutations can be created in one strain sequentially.

Examples of non-transgenic auxotrophic lines are provided in Table 1.

TABLE 1
Auxotrophic Mutations

| Genotype | Mutant classes |
|---|---|
| | Amino acid biosynthesis single gene mutants^{a} |
| argD, argG, leuB, | Arginine, leucine, proline, threonine, and tryptophan auxotrophy, respectively |
| leuC, proC, thrA, | |
| trpE | |
| | Nucleotide biosynthesis single gene mutants^{a} |
| guaB, purC, pyrB | Guanine, purine, and pyrimidine auxotrophy, respectively |
| | Sugar utilization single gene mutants |
| gapA^{b} | GAPDH mutant; sugar utilization through both glycolysis and the pentose |
| | phosphate shunt is blocked |
| ptsH^{a}, ptsI^{a} | Phosphoenolpyruvate-dependent sugar phosphotransferase system [PTS] |
| | disabled, causing defect in uptake of multiple sugars |
| | Nitrogen utilization single gene mutants^{a} |
| gltA, gltB | GOGAT mutant defective in utilization of multiple amino acids and high-affinity |
| | NH₄⁺ uptake; glutamate auxotrophy |
| | Other biochemical pathway single gene mutants |
| foxR^{b} | Defective in high affinity, siderophore-based iron uptake system |
| glmS^{a} | Glucosamine auxotrophy (defect in hexosamine metabolism and glycosylation) |
| thiC^{b} | Thiamine auxotrophy (vitamin B₁) |
| | Mutants affecting multiple major biochemical pathways |
| carA^{a}, carB^{a} | Carbamoylphosphate synthase mutants; arginine and pyrimidines auxotrophy |
| argD guaB^{b} | Double mutant combining arginine and purine (guanine) auxotrophy |
| argD leuB^{b} | Double mutant combining arginine and leucine auxotrophy |
| leuB thiC thrB^{b} | Triple mutant combining leucine, thiamine, and threonine auxotrophy |
| | Virulence factor mutant |
| rpoN^{c} | Transcription factor mutant defective in expression of multiple essential virulence |
| | factors including amylovoran and the T3SS, avirulent on apple fruitlets and trees |

^{a}Mutants previously isolated and characterized in Klee et al., “Extragenic Suppression of Elongation Factor P Gene Mutant Phenotypes in Erwinia amylovora,” J. Bacteriol. 201: e00722-18 (2019), which is hereby incorporated by reference in its entirety.
^{b}Mutants to be created by targeted deletion.
^{c}Mutant previously isolated and characterized in Ramos et al., “The Fire Blight Pathogen Erwinia amylovora Requires the rpoN Gene for Pathogenicity in Apple,” Mol. Plant Pathol. 14: 838-843 (2013), which is hereby incorporated by reference in its entirety).

### Example 3—Fire Blight Field Test of the Leucine-1 Auxotroph in Apple Trees

The pathogenic behavior of E. amylovora auxotrophs in field applications on blooming trees was completely unknown and untested at the time of the field test described here. A field trial conducted by Washington State University took place at the Columbia View Research Farm in Wenatchee, WA. Trees were selected based on sufficient bloom (100+ flower clusters on lower branches to facilitate evaluation). The ‘Red Delicious’ apple tree cultivar was used. Trees were marked as plots in randomized complete block design.

The products were applied to trees using a Stihl SR420 mist blower backpack sprayer until runoff, equivalent to approximately 100 gallons per acre (0.5 gallons per tree). Commercial products were applied according to label instructions: oxytetracycline standard (FireLine 17%), streptomycin standard (FireWall 17%), and organic standard (Blossom Protect 1.24 lb/100 gal+Buffer Protect 8.75 lb/100 gal+Previsto 3 qt/100 gal). The Leucine-1 auxotroph was applied at 10⁹ cell/ml in water. To produce auxotroph cells for testing, the Leucine-1 auxotroph was grown on LB medium plates for 48 hours and sufficient quantities were scraped from the plates and suspended in water to create a 10⁹ cell/ml suspension, which was verified by serial dilution plating and colony counting. The concentration of Leucine-1 auxotrophic cells applied in the Washington trial was 1×10⁹ cells/ml, which was determined to be a suboptimal concentration for auxotrophic bacteria to inhibit virulent E. amylovora growth based on the detached apple, crabapple, and hawthorn flower studies on a different transgenic Tn5-induced auxotroph (see Klee et al., “Erwinia amylovora Auxotrophic Mutant Exometabolomics and Virulence on Apples,” Applied Envir. Micro. 85: e00935-19 (2019), which is hereby incorporated by reference in its entirety).

Apple trees were treated at 100% king bloom in the morning with the various treatments. The following morning, trees were inoculated with virulent Erwinia amylovora at 10⁶ cells/ml using a 1-liter sprayer to lightly wet each flower cluster. Whole trees (100+ clusters) were inoculated in this manner. Trees were visually evaluated weekly following inoculation for flower cluster infection and blossom blight symptoms. Fruit was also evaluated for skin russet in July. Statistically significant differences were assessed using analysis of variance (ANOVA) and multiple t-test comparisons using SAS version 9.4.

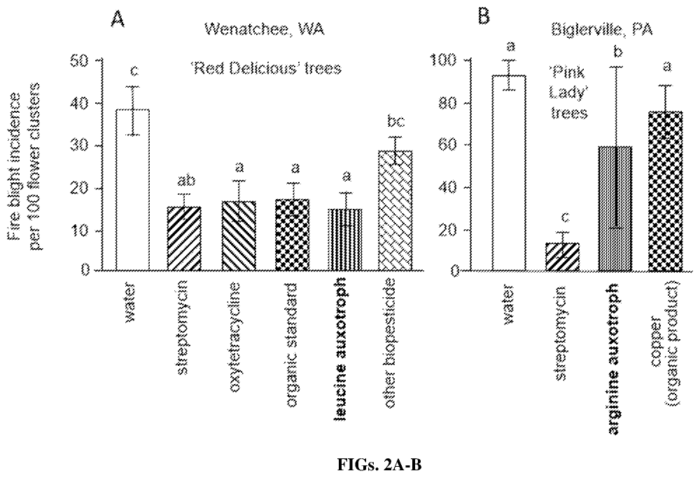

FIG. 2A is a chart showing the results of orchard application of a non-pathogenic UV-induced E. amylovora Leucine-1 auxotrophic mutant to ‘Red Delicious’ apple trees at bloom time, in which the UV-induced E. amylovora Leucine-1 auxotrophic mutant was shown to reduce fire blight incidence as effectively as antibiotics and a standard organic product. The trees were sprayed at 100% open king blooms. The products were applied at label rates, and the E. amylovora Leucine-1 auxotroph was applied at 10⁹ CFU/ml in water. Inoculation with pathogenic E. amylovora was performed the day after product application at 10⁶ CFU/ml wild type E. amylovora. Bars sharing the same letter have no statistically significant difference using Fishers t-test (LSD) at p=0.05. As shown in FIG. 2A, the E. amylovora Leucine-1 auxotroph was as effective at blocking fire blight disease as streptomycin, oxytetracycline, the organic standard, and other biological agent.

Surprisingly, the 10⁹ cell/ml suspension concentration of the Leucine-1 auxotroph performed very well in Washington in 2021 under field conditions, making the application of the Leucine-1 auxotroph more practically useful. As described in Example 5, an even lower concentration of 2×10⁸ CFU/ml of the Leucine-1 auxotroph was found to be effective when the amino acid leucine was included in the formulation.

The field application in Washington also did not include a surfactant (wetting agent), the Leucine-1 auxotroph was applied in water only. Prior studies in the laboratory on detached flowers had included the surfactant Tween®-80. Since biocontrols are generally applied with a wetting agent to improve adhesion to hydrophobic (waxy) plant surfaces, it was surprising that the Leucine-1 auxotroph was so efficacious at blocking fire blight disease despite being applied in pure water. Lack of a wetting agent in the mixture was expected to reduce or potentially eliminate the efficacy of the application for fire blight disease prevention.

Russetting in the Leucine-1 auxotroph treated plants and controls was measured by examining 25 fruits per tree using a 0-15 point scale for russet. The Leucine-1 auxotroph treated tree fruit had an average score of 0.05 (barely detectable), which was similar to that seen with the antibiotic treated tree fruits (0.06). Organic-compatible products had higher scores, 0.6 to 0.7. However, all of these were below the threshold for commercial quality downgrades, which is 3.0. Fruit from water-treated trees had 0.00 russet score, but had a lot of fire blight loss of fruit. It is not uncommon for fire blight control agents to cause some russetting, for example copper. Russet observed in fruits from trees treated with the Leucine-1 auxotroph was minimal and not commercially relevant.

### Example 4—Fire Blight Field Test of an Arginine-1 Auxotroph in Apple Trees

The inhibition of virulent E. amylovora growth on host flowers was not complete for the transgenic Tn5-induced arginine auxotroph reported in Klee et al., “Erwinia amylovora Auxotrophic Mutant Exometabolomics and Virulence on Apples,” Applied Envir. Micro. 85: e00935-19 (2019), which is hereby incorporated by reference in its entirety. The virulent E. amylovora cells multiplied by 1-2 orders of magnitude in all experiments using E. amylovora auxotrophs. This raised uncertainty of whether the growth inhibition afforded by E. amylovora auxotrophs would be sufficient to block the disease-causing ability of virulent E. amylovora in field grown trees. Unlike antibiotics, E. amylovora auxotrophs do not kill the virulent E. amylovora cells, but merely inhibit their reproduction to some degree. Therefore, E. amylovora auxotrophs might fail to block fire blight disease development, despite moderately inhibiting the growth of E. amylovora.

A non-transgenic, UV-induced arginine E. amylovora auxotrophic mutant, Arginine-1, was grown in sufficient quantities for field sprayer application at the Pennsylvania State Huck Institutes of the Life Sciences CSL Behring Fermentation Facility. 100-liter batches were grown overnight in LB liquid medium in a Sartorius Biostat D-DCU vessel and bacteria separated using a continuous flow Sharples AS-16 Centrifuge. A trial run in April 2021, with the Arginine-1 auxotroph produced nearly 400 mg of bacterial wet-cell paste, sufficient to produce over 20 liters of 10¹⁰ CFU/ml inoculum, which was more than sufficient for sprayer application. The wet-cell paste did not lose viability when stored in the refrigerator at 4° C. for at least one month, as determined by serial dilution plating. The UV-induced E. amylovora Arginine-1 auxotroph retained its auxotrophic characteristics after culturing in 100 liter volume and storage under refrigeration. A freeze drying method was used to preserve E. amylovora inoculum developed at Washington State University for field testing.

A field trial conducted by Pennsylvania State University was performed on the apple tree variety ‘Pink Lady’. Five replicates of single trees were treated and evaluated. Treatments including the E. amylovora non-transgenic Arginine-1 auxotrophic strain, Firewall, Firewall+Regulid, Firewall+LI700, and Aliette were applied at 50% bloom in the early morning followed by treatment with pathogenic Erwinia amylovora suspension in the late afternoon of the same day. Trees were rated for positive infection at 2 weeks after inoculation. Shoot blight was rated about two months later. The percent blossom blight infection is shown in Table 2. Different letters indicate significant differences between treatments.

TABLE 2
Fire Blight Blossom Blight Infection in PA Apple Field Trial

| Treatment | % Infection |
|---|---|
| Water Only | 94.5% a |
| Fire Wall Only | 11.1% c |
| Fire Wall + Regulid | 10.8% c |
| Fire Wall + LI700 | 18.7% bc |
| Aliette | 75.9% a |
| E. amylovora Arg-1 auxotroph + Nu-Film P | 60.6% b |

As shown in Table 2, the Arginine-1 auxotroph provided statistically significant protection from blossom blight. The water control had 95% blossom blight incidence, the auxotroph had 60% incidence, an organic product had 76% (not statistically different from water control), and streptomycin had 11% incidence. The PA trial had a more humid environment, higher rainfall, extremely high disease pressure, and a different apple cultivar (Pink Lady) compared to the WA trial. The PA trial also included the use of a surfactant with unknown interaction with the bacteria (Nu-Film P).

A subsequent trial of the Arginine-1 auxotroph in apple trees in Pennsylvania was unsuccessful because the auxotroph was believed to be applied too early, when the weather was too cold for fire blight to develop. In this trial, the water control had 76.0% disease incidence while the Arginine-1 auxotroph treatment showed 66.8% disease incidence, which was not a statistically significant difference.

### Example 5—Fire Blight Field Test of the Leucine-1 Auxotroph in Pear Trees

Pears are much more susceptible to fire blight than apples, and the ‘d'Anjou’ pear cultivar is particularly susceptible among pears. For example, pears are not grown commercially in Pennsylvania due to the extreme fire blight disease pressures, while apples can be grown commercially in Pennsylvania.

A 0.5 ha research block of 4-yr-old ‘d'Anjou’ pears at Washington State University (WSU) Columbia View Orchard in Wenatchee, WA was used for this trial in 2022. The experiment was arranged in a randomized complete block with five single tree replicates. Products were applied to the whole tree according to manufacturer recommendations using a Stihl SR420 mist blower backpack sprayer. Products were applied to wet, near dripping at 0.2-0.4 gal/tree (100 gal/A). Application dates were: 17 April (1), 20 April (2), 22 April (3), 23 April (4), 25 April (5), 26 April (6), 27 April (7), 29 April (8), 6 May (9), 13 May (10). At 100% bloom (of the king blooms), 22 April.

After product application, virulent Erwinia amylovora (Ea153N) was applied at 1×10⁶ CFU/ml (verified at 17×10⁶ CFU/ml) to lightly wet each cluster. Trees were visually evaluated for flower cluster infection weekly from when symptoms became visible 10 days after inoculation for 3 weeks and infection counts summed across all dates. Fruit was evaluated for fruit skin marking on a 0-15 scale. E. amylovora was enumerated 1, 4, and 7 days after inoculation from a bulk sample of 10 flowers per tree. Clusters were sonicated in sterile water for 3 minutes and a 10 μl sample of the wash and two 1:100 dilutions were spread on nutrient agar amended with nalidixic acid (50 μg/ml) and cycloheximide (50 μg/ml) to selectively enumerate E. amylovora (Ea153N). Statistical analysis was performed using general linear mixed models (GLIMMIX) analysis of variance ANOVA, and multiple means comparison (LSD) SAS v 9.4. Environmental conditions during bloom (14-26 April) ranged from an average maximum temperature of 57.9° F. and minimum of 37.5° F. with 52.6% average humidity and with snow on open blooms on 14 April. A precipitation event (0.11 in) occurred on 20 April the evening after 90% full bloom sprays were applied. Multiple rain and wind events and cool spring weather impacted infection pressure and treatment timings.

TABLE 3
Results of Fire Blight Blossom Blight Infection in the WSU Pear Tree Field Trial

| | Amount | | | Infections | Fruit |
|---|---|---|---|---|---|
| Treatment | per 100 gal | | Timing | per 100 Clusters | Russet |
| Streptomycin standard | 8 | oz | 3 | 4.4 ± 1.2 d | 0.2 |
| (Firewall 50WP)^{x} | | | | | |
| Oxytetracycline standard | 9 | oz | 3, 6 | 15.7 ± 4.8 bc | 0.2 |
| (Fireline 45WP) | | | | | |
| Organic standard apple | 1.25 1b + 5 lb | | 1, 2, 3, 6 | 11.0 ± 4.0 cd | 1.1 |
| Blossom Protect + Buffer | 3 qt | | | | |
| Protect | | | | | |
| Previsto | | | | | |
| Organic standard pear | 1.25 1b + 5 1b | | 1, 3, 4, 6 | 16.9 ± 2.6 abc | 0.6 |
| Blossom Protect + Buffer | 96 fl oz | | | | |
| Protect | | | | | |
| Serenade Aso | | | | | |
| PSU1 | 200 | g | 2, 4, 6 | 25.5 ± 3.2 ab | 0.2 |
| PSU2 | 1.7 | kg | 2, 4, 6 | 18.3 ± 6.5 abc | 0.3 |
| PSU3 | 500 | g | 2, 4, 6 | 15.0 ± 3.5 bc | 0.2 |

| | | | | |
|---|---|---|---|---|
| Water Treated Check | NA | 3, 4, 6 | 35.5 ± 5.4 a | 0.3 |

Table 3 shows the effect of Penn State biological treatments applied to pear, cultivar ‘d'Anjou’ on infection of Erwinia amylovora in pear blossoms in Wenatchee, WA. Treatments followed by the same letter in the column labeled “Infections per 100 clusters” were not significantly different at P=0.05 Fisher's T test (LSD). Non-transformed means are shown for infections per 100 clusters, which were transformed log (x+1) prior to analysis of variance. Included in the trial as a comparison and as “treated checks” were Fire Wall 50WP (streptomycin sulfate) and FireLine 45WP (oxytetracycline). FireWall and FireLine were amended with Regulaid at 16 fl. oz. per 100 gallons and buffered to 5.6 pH. Regulaid is a nonionic spreader-activator for use in improving the wetting effectiveness, uniform spray coverage, and penetration of foliar applied products.

Two conventional organic standard products were also tested in the trial. The first was Organic standard apple Blossom Protect+Buffer Protect Previsto, and the second was Organic standard pear Blossom Protect+Buffer Protect Serenade ASO. Serenade ASO includes Bacillus subtilis. Previsto includes copper ions in a natural polymer matrix.

An untreated-inoculated check treatment was included in the trial. The Leucine-1 strain used for the pear study of 2022 was the same as that used for the apple study in Washington in 2021. For the PSU1 treatment, Erwinia amylovora Leucine-1 strain was provided as a wet cell paste at 10⁹ CFU/ml, which was gently resuspended at 2 g per gallon. For the PSU2 treatment, Erwinia amylovora 87-70 “Leucine-1” strain was provided as a dry powder at 10⁹ CFU/ml. The formulation of the dry powder used in the 2022 pear trial was by weight 58% cells, 34% milk product (nonfat), and 8% glycerol. For the PSU3 treatment, Erwinia amylovora 87-70 “Leucine-1” strain was provided as a wet cell paste at 2×10⁸ CFU/ml plus 10 mM leucine.

Inoculation of virulent Erwinia amylovora was conducted on the evening of 22 April at full bloom (of king blooms) using a suspension of freeze-dried cells of Erwinia amylovora strain Ea153 (streptomycin and oxytetracycline sensitive strain) prepared at 1×10⁶ CFU/ml (verified at 17×10⁶ CFU/ml).

The numbers in the ‘Timing’ column of Table 3 correspond to the following: 1: 70% bloom, 2: 90% bloom, 3: morning before evening inoculation (full bloom), 4: morning after inoculation, 5:2 days after inoculation, 6:3 days after inoculation (petal fall), 7:4 days after inoculation, 8:6 days after inoculation, 9:2 weeks after inoculation, 10:3 weeks after inoculation.

Of the Pennsylvania State University biological product formulations, the PSU3 formulation applied alone provided approximately 58% relative control significantly different than the water treated check, and comparable to organic and oxytetracycline standards. The addition of supplemental leucine to treatment was hypothesized to allow the microbe to multiply and colonize the plant more effectively for a limited number of replication cycles. The PSU1 and PSU2 treatments also reduced blossom infections (Table 3), but the values were not significantly different than the water-treated check.

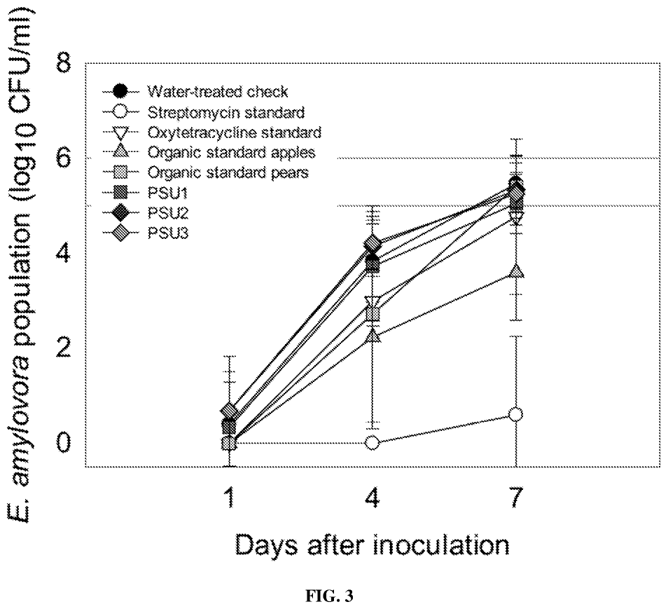

Population levels of E. amylovora strain ‘153N’ on flowers were not reduced when Penn State treatments were applied at 90% bloom, the morning after inoculation and 3 days after inoculation, while they were reduced with the application of the organic standard for apples and both antibiotic controls (FIG. 3).

For fruit russetting, fruit marking was rated from an average of 25 fruit per tree. In this trial, less than 25 fruit were often present. Fruit were rated on a 0 to 15 scale where ratings below 3 indicate no commercial downgrades. No statistical differences were observed between treatments.

The PSU1 treatment contained the same rate of the Leucine-1 active ingredient that was effective in protecting apples from fire blight in Washington in the 2021 tests, but it provided only a relatively small reduction in fire blight disease in the pear trial. This result may reflect the extreme susceptibility of pears to fire blight. For PSU2, the addition of a protein adjuvant/carrier in the form of a crude complete protein (e.g., milk) enhanced the activity of the Leucine-1 product. Surprisingly in PSU3, the addition of a specific free amino acid, leucine, boosted product performance to match that of the antibiotic even though the concentration of Leucine-1 applied to the trees was less. Overall, the inclusion of adjuvant/carrier or leucine improved the auxotrophic product effectiveness.

Table 4 shows the environmental conditions during bloom for the pear trial.

TABLE 4
Environmental Conditions During Bloom

| | Min Air | Max Air | | | |
|---|---|---|---|---|---|
| Date | Temp ° F. | Temp ° F. | Humidity | Leaf wetness | Wind speed |
| 14-Apr | 31.8 | 45.7 | 73.7 | 0.26 | 5.3 |
| 15-Apr | 28.9 | 50.3 | 62.4 | 0 | 3.9 |
| 16-Apr | 34.1 | 49.4 | 59 | 0 | 5 |
| 17-Apr | 35.3 | 55.6 | 41.5 | 0 | 5.5 |
| 18-Apr | 40.1 | 46.4 | 63.1 | 0 | 7.2 |
| 19-Apr | 37.9 | 58.3 | 43.6 | 0 | 5.2 |
| 20-Apr | 32.7 | 53.0 | 58.9 | 0.11 | 3.6 |
| 21-Apr | 42.5 | 61.0 | 62.9 | 0.06 | 4.7 |
| 22-Apr | 36.3 | 65.6 | 50.2 | 0 | 4.1 |
| 23-Apr | 40.8 | 68.2 | 41.5 | 0 | 3.7 |
| 24-Apr | 38.5 | 70.3 | 43.8 | 0 | 4.6 |
| 25-Apr | 45.8 | 67.5 | 47 | 0 | 4.7 |
| 26-Apr | 43.2 | 61.9 | 35.9 | 0 | 4.5 |
| 27-Apr | 39.7 | 62.6 | 38.8 | 0 | 4 |
| 28-Apr | 37.4 | 61.7 | 42.5 | 0 | 3.7 |
| 29-Apr | 44.6 | 65.8 | 35.6 | 0 | 5.9 |
| 30-Apr | 45.4 | 57.2 | 65.3 | 0.07 | 4.7 |
| 1-May | 39.4 | 71.6 | 56.3 | 0 | 6.5 |
| 2-May | 48.3 | 58.7 | 69.8 | 0.13 | 4.1 |
| 3-May | 45.3 | 72.6 | 48.9 | 0 | 4.2 |
| 4-May | 51.7 | 72.7 | 51.3 | 0 | 4.2 |

TABLE 5
Exemplary Nucleotide Sequences

| SEQ | | |
|---|---|---|
| ID | | |
| NO: | Gene | Sequence |
| 1 | Erwinia | ATGAGCCAAC AAGTCATTAT TTTTGATACC ACTCTGCGCG ATGGCGAGCA |
| | amylovora | GGCATTACAG GCCAGTCTGA GTGTGAAAGA AAAGCTGCAG ATTGCCCTGG |
| | leu A | CGCTGGAGCG GATGGGGGTT GATGTCATGG AGGTCGGTTT CCCGGTCTCT |
| | | TCCCCCGGGG ATTTTGAATC GGTACAAACC ATTGCCCGCC AGATCAAGAG |
| | | CAGCCGGGTG TGTGGCCTGG CACGCTGTGT GGAAAAGGAT ATCGACGCGG |
| | | CTTATGAAGC GCTGCGCGTG GCGGATGCCT ATCGTATTCA CACCTTCCTG |
| | | GCGACCTCAC CGATGCATAT CGCCACCAAA CTGCGCAGCA CCCTGCCGGA |
| | | AGTGATTGAA CGCGCCGTGA AGATGATTAA GCGCGCACGC AATTACACCG |
| | | ACGACGTGGA GTTCTCCTGC GAAGACGGCG GTCGTACCCC GATTGACGAC |
| | | CTGTGCCGCG TGGTGGAAGC CGCGATTAAC GCCGGTGCCA CCACCATCAA |
| | | TATCCCCGAT ACCGTGGGTT ATACCTTACC TTACGAATAC GCCAGTATTT |
| | | TCAGCGCGCT GCGCGCACGC GTGCCGAATA TTGATAAAGC GATCCTGTCG |
| | | GTGCATACCC ACGACGATCT GGGCATGGCC GTCGGTAATG CGCTGGCGGC |
| | | GGTGAATGCC GGTGCGCGTC AGATCGAAGG GGCAATGAAC GGACTGGGTG |
| | | AACGCGCCGG TAACTGCGCG CTGGAAGAGA CCATTATGGC GATCAAAACT |
| | | CGTGGTCAGC TGATGAACGT GCATACGCGC ATCCATCACC AGGAAATCTA |
| | | CCGCACCTGT CAGACCGTGA GCAAAATCTG CAATATGCCG ATCCCGGCGC |
| | | ATAAGGCAAT TATTGGATCC AACGCATTTG CGCACTCTTC CGGTATTCAC |
| | | CAGGACGGCG TACTGAAAAA CCGCGAAAAC TACGAGATCC TGACGCCGGA |
| | | ATCGATCGGC CTGAAACAGG TACAGTTGAA CCTGACCTCG CGTTCCGGCC |
| | | GCGCCGCCGT GAAACACCGC ATGGAAGAGA TGGGCTACGG CGAAGACGAA |
| | | TACAACATGG ACAGGCTGTA CGACGCGTTC AAGGCGCTGG CCGATAAAAA |
| | | AGGCCAGGTG TTTGATTACG ATCTGGAGGC GCTGGCATTT ATCAGTCAAC |
| | | AGTCGGAAGA ACCGGAATAC TTCAGGCTGG ATACGTTCAA CGTGCAGTCC |
| | | GGCTCCAGCG TCATTGCCAC CGCCACCGTG CAGCTGTGCT GCGGCGACCA |
| | | ACAGAAAACC GAAGCCGCTA CCGGAAACGG TCCGGTCGAC GCGGTTTATC |
| | | AGGCAATAAA CCGTATGACT GAATTCAACG CCGAGCTGAT CAGTTATCAG |
| | | TTGACCGCCA AAGGCCACGG TAAAGACGCG CTGGGCCAGG TCGATATTGT |
| | | GGTGCAGTAC AACGGCCGCA AATTCCACGG CGTCGGCCTG GCGACCGATA |
| | | TCGTCGAATC CTCGGCAAAA GCGATGGTAA ACGCGCTGAA CACCATCTGG |
| | | CGCGCGCGTC AGGTTGAGCA GGAATTACAG CGTAAGTCCC AGGTCAAAGA |
| | | TCACAAGGAA ACCGTATAA |
| 2 | Erwinia | ATGTCCCGTA CTTACCATAT TGCAGTGTTA CCCGGTGACG GCATCGGCCC |
| | amylovora | GGAAGTGATG GCTCAGGCCA GCAAGGTGCT CGACGCTATC CGCCAGCGCT |
| | leuB | TTGATCTGCG TATCACTACC AGCGAATACG ACGTCGGCGG TATTGCTATC |
| | | GACCGTCACG GCGTGCCGCT GCCGGACAGC ACGGTAAAAG GCTGTGAGCA |
| | | GGCCGATGCC ATCCTGTTTG GCTCGGTCGG CGGGCCAAAG TGGGAACATC |
| | | TGCCCCCGGC AGAACAGCCC GAACGCGGCG CGCTGCTGCC GCTGCGTAAA |
| | | CACTTTAAGC TGTTCAGCAA CCTGCGTCCG GCCAGCCTGT ACCAGGGGCT |
| | | GGAGGATTTT TGCCCGCTGC GTCGGGATAT TGCCGATCGC GGCTTCGATA |
| | | TTCTGGTGGT GCGCGAGCTG ACCGGCGGGA TCTACTTTGG TCAGCCGAAG |
| | | GGCCGCGAAG GCAGCGGGAT GCATGAACGC GCCTTTGATA CCGAAGTCTA |
| | | TTACCGCTTT GAAATCGAGC GCATTGCACG TATTGCCTTT GAATCAGCGC |
| | | GTAAACGCCG TCACAAAGTG ACCTCGATTG ACAAAGCCAA CGTGCTGCAA |
| | | TCCTCCATTC TCTGGCGTGA AATCGTGAGT GAGGTGGCAC AGGATTACCC |
| | | GGATGTTGCG TTAAGCCATA TGTATATCGA CAACGCCACC ATGCAGCTAA |
| | | TCAAAGCGCC ATCGCAGTTC GACGTGATGC TGTGCTCGAA TCTGTTCGGC |
| | | GATATCCTGT CGGATGAATG CGCGATGATC ACCGGTTCAA TGGGCATGCT |
| | | GCCTTCCGCC AGCCTTAACG AGCAGGGCTT TGGCCTGTTC GAACCGGCAG |
| | | GCGGCTCCGC ACCGGATATC GCCGGTAAAA ACATCGCCAA CCCGGTGGCG |
| | | CAAATTCTGT CGCTGGCGCT GCTGCTGCGC TACAGTCTGG ATGCAGCCGC |
| | | AGCGGCCGAC GCCATTGAGC GGGCGATCAA CCGGGCGCTG GAAGAGGGTC |
| | | ACCGTACCGG AGACCTGGCC GGAAACGGCA AGGCCATCGG CACCGATGAA |
| | | ATGGGCAGCG TGATTGCCCG ATTTATTCGC GAAGCATAA |
| 3 | Erwinia | ATGAAGACAC TGTACGAGAA GCTGTTTGAT GCGCATGTCG TGCATGAAAC |
| | amylovora | CCACGGCGAA ACGCCGCTGA TTTATATCGA CAGGCACCTG GTGCATGAAG |
| | leuC | TGACCTCTGC CCAGGCCTTT GACGGTCTGC GCGCCCACGG TCGTGCGGTG |
| | | CGCCAGCCGT CGAAAACCTT TGCCACCATG GATCACAACG TGTCTACCCA |
| | | GTCGCGCGAT ATTAACGCCA GCGGCGAAAT GGCGCGCATC CAGATGTCAA |
| | | CGCTTGTGAA AAACTGTGAA GCCTTTGGCG TACAGCTGTT TGACCTCAGT |
| | | CACCCGTTCC AGGGGATCGT CCATGTGATG GGGCCGGAAC AGGGGCTGTC |
| | | GCTGCCCGGC ATGACCCTCG TCTGCGGTGA TTCCCACACA TCCACCCACG |
| | | GCGCTCTGGG GGCGCTGGCG TTCGGCATCG GCACCTCGGA AGTGGAACAC |
| | | GTTCTGGCTA CCCAGACACT GAAGCAGTCA CGCGCCAAAA CCATGAAGAT |
| | | CGAAGTGCGT GGTGACACGG CGGCGGGCAT CACCGCCAAA GATATTGCGC |
| | | TGGCGATTAT CGGCAAAACC GGCAGCGGCG GTGGTAACGG TCACGTTGTC |
| | | GAATTCTGCG GTGCGGCGGT GGAAGCCCTG AGCATGGAAG GCCGCATGAC |
| | | GCTGTGTAAT ATGGCGATTG AGCTGGGCGC TAAGGCCGGA TTAATCGCCC |
| | | CGGACGACAC CACGTTCAGC TACGTAAAAG GCCGTCGTTA TGCCCCCAAA |
| | | GAGGCCGAAT GGCAGCAGGC GGTCGACTAC TGGCGCACCC TGCACACCGA |
| | | TGAGGGCGCG CGCTTTGACA AGGTGGTGAT TATCGATGCT GCGGCCATTG |
| | | CCCCCCAGGT AACCTGGGGA ACCCATCCCG GACAGGTGAT CGCCGTCAGC |
| | | GATCCGATCC CCGATCCGGC CTCTTTCAGC GATCCGCTGG AGCGCGCCTC |
| | | GGCGGAAAAA GCGCTGGCCT ATATGGGGCT CAAGCCCGGT ATCCGTCTGA |
| | | CCGACGTCCC CATCGATAAG GTGTTTATCG GTTCCTGTAC CAACTCGCGC |
| | | ATTGAAGATC TGCGTGCGGC GGCAGCAATC GTCAAAGGGC AAAAAATTGC |
| | | CAAGGGCGTA CAGGGTTATG TGGTGCCAGG TTCCGGCCCG GTGAAGGCGC |
| | | AGGCGGAAGC GGAAGGGCTG GATCGCATCT TTATTGCTGC CGGATTTGAA |
| | | TGGCGTTTGC CGGGCTGTTC AATGTGCCTG GCGATGAATA ATGACCGCCT |
| | | GCAGCCGGGT GAACGCTGTG CGTCAACCAG CAACCGTAAC TTTGAAGGCC |
| | | GCCAGGGGCG CGGCGGGCTT ACTCATCTGG TCAGTCCGGC GATGGCGGCA |
| | | GCGGCAGCGG TAGCCGGTCA CTTTGCCGAT ATCCGCGAGC TGGCGTAA |
| 4 | Erwinia | ATGAGTCAGA AATATCGCGT GTTTATCGAC GGCCAGGCAG GCACGACCGG |
| | amylovora | ATTGCAGATC CAGAAACGAT TAGCCCGTCA CCCTCAGATT GAAATGCTAC |
| | argC | AGATTGATGA AGCACAGCGC AAAGATGCCG CCGCCCGTCA GGCGCTGATG |
| | | AAACAGGCTG ATGTCAGTAT CCTTTGCCTG CCGGATGCCG CGGCGCGGGA |
| | | AGCGGTAGCG CTGGCCGACG AGGTGGGTGC ACGCGTGCTG GATGCCAGTT |
| | | CCGCTCACCG GGTGGATGCC GGCTGGGTAT ACGGCCTGCC AGAGCTTGCT |
| | | CCTGAACAAC GTGATAAAAT CAGCGGCGCC AGCCATGTAT CTAACCCGGG |
| | | CTGTTATGCT ACGGGGGCTA TTGCCCTGCT GGCGCCACTG ATTCGAAATG |
| | | GTCTGTTGAA GCCAGACTGC CAGCTGGCAA TCAACGCGAT TTCCGGTTAC |
| | | AGCGGCGGCG GCAAGCAGAT GATCGAGCGG TATTGCCAGG CGGGCACCGG |
| | | TTATGCTGCT TACGGTCTGG ACTTTGATCA TAAACATATT AAAGAGATTA |
| | | AGGCGTGGAG CGGTCTGAGT AGCCGGCCCA TTTTACAACC TGCGGTCGGT |
| | | GACTACGCAC AGGGCATGCT GGTGTTCATC CCTTTAAATG GCATTGACAG |
| | | CGAGCCGTTA CAGCAGGCGC TGGCAGACTA TTACGCCGGC CAACTGTTTA |
| | | TTAAGGTCAA TCCCCTGAAC GCCATGGATG AGCACAGCGC GCCCTATCTG |
| | | CTGCCGGAAG CGCTGAATAA TACCAATCAA TTAGAGCTGT TCGTCTTCAG |
| | | CCACCCACAG CAGCGTCAGT GCATTTTAGC ATCACGGCTG GATAACCTGG |
| | | GTAAAGGGGC TTCGGGTGCC GCCGTGCAGA ACCTGAACCT GATGCTCGGT |
| | | CTGGCAGAAG AGACCTGCGT TAACCTCTGA |
| 5 | Erwinia | ATGGCGGCGG AAAAAATAGC GGTAACGCGG GCGACATTTG ACGAGGTAAT |
| | amylovora | TTTGCCTGTT TATGCACCAG CGCAGTTTGT GCCGGTCAAA GGCAAAGGCA |
| | argD | GCCGTATCTG GGACCAGCAG GGTAAAGAGT ATATCGATTT CTCCGGTGGG |
| | | ATTGCGGTTA CCGCGCTGGG CCACTGCCAT CCGGCGCTGG TGGAAGCGTT |
| | | AAAAACCCAG GGCGAAACCC TGTGGCACAC CAGTAACGTT TTCACCAATG |
| | | AGCCAGCCTT GCGCCTGGCG CGTAAACTGA TCGATGCCAC TTTCGCCGAG |
| | | CGCGTATTCT TTGCCAACTC CGGCGCGGAA GCCAACGAAG CGGCTTTCAA |
| | | GCTGGCGCGT TACTACGCTT CACAGCGCCA CAGCCCGTAT AAAAGCAAAA |
| | | TTATCGCTTT CCATAATGCG TTTCATGGCC GCACGCTGTT TACCGTTTCC |
| | | GTCGGCGGGC AGCCAAAATA CTCCGATGGC TTTGGGCCAA AACCGGCCGA |
| | | TATTGTCCAT GTGCCCTTTA ACGATCTGGC GGCGGTCAAA GCGGTCATCG |
| | | ACGATCATAC CTGTGCGATT GTCGTCGAGC CGATCCAGGG GGAGGGCGGC |
| | | GTGGTACCGG CCACTGAGGA ATTCATGCAG GGGCTGCGTC AGCTGTGTGA |
| | | TGAACATCAT GCGCTGCTGG TGATGGACGA AGTACAGAGC GGCATGGGCC |
| | | GTAGCGGCAA GCTGTTTGCC TACGAGCACT ACGGCGTGCA GCCAGATATT |
| | | GTCACCAGCG CGAAAGCACT CGGCGGCGGC TTCCCGGTCA GCGCGATGCT |
| | | GACCACCAAC GCTATTGCCT CAGTGATGGC CCCGGGCGTG CACGGCACCA |
| | | CCTACGGCGG CAATCCGTTG GCCTGTGCGG TGGCGGAAGC GGCGCTGGAC |
| | | ATTATCAATA CGCCGGAAGT GCTTGACGGC GTGGCCGCGC GGCGGCAGCT |
| | | GTTTGTTGAT GCGCTACAGT CGCTGAATAC CAGGCGGGAT CTGTTCAGCG |
| | | ATATCCGTGG TAAAGGGCTG CTGATTGGTG CAGCGCTGAA GCCACAGTAC |
| | | GCTGATAAAG CCCGCGATAT CCTTAACGCG GCGGCGGCCG AAGGGGTGAT |
| | | GGTGCTGGTC GCCGGCAGCG GCGTGATGCG CTTCGCGCCA TCGCTGTTGA |
| | | TTGAGCCTGT CGATATTGCC GAAGGTATGG AGCGCTTTGC TGCGGCCGTC |
| | | GAGAAGGTGC TGGCGTAG |
| 6 | Erwinia | GTGAAGACAA AATTACCCAA TTTTATTGAG ATTTACCGCC AGTTGATTGC |
| | amylovora | AACGCCATCA ATCAGTGCTA CCGACAGCGC GCTCGATCAA AGCAATGAAA |
| | argE | CTTTAATCAA TTTGCTCGGC GGCTGGTTCC GCGATCTTGG CTTCAGCGTT |
| | | GAGGTACAGC CGGTTCCCGG CACGCGCAAT AAATTCAATA TGCTGGCCAG |
| | | GAGCGGACAG GGTGCCGGCG GCCTGCTGCT GGCGGGCCAT ACCGACACCG |
| | | TTCCCTTTGA CGATGGCCGC TGGACGCGCG ATCCCTTCAC GCTTAACGAG |
| | | CATGACAATA AGCTGTACGG ACTCGGCACC GCCGATATGA AAGGTTTCTT |
| | | CGCCTTTATT TTAGACACGC TGCGTGATGT TGACCTGTCT GCGCTGAAAA |
| | | AACCGCTGTA CGTTCTGGCC ACCGCCGACG AAGAAACGAC GATGGCCGGT |
| | | GCGAAATTCT TCTCTGAATC CACCGCTCTG CGCCCGGACT GCGCCATCAT |
| | | TGGCGAACCA ACCTCGCTAA AACCGGTGCG CGCGCATAAA GGCCATATCT |
| | | CAAACGTCAT CCGTATTCAG GGGCAGTCCG GCCACTCCAG CGATCCGGGC |
| | | CGCGGCGTCA ACGCCATTGA ACTGATGCAC GAATCCATCA CCCGGTTAAC |
| | | GGGGCTGCGC AACACGCTGA AAGAGCGCTA CCATCACGCG GGTTTTGCCA |
| | | TTCCTTACCC AACGATGAAT TTTGGCCACA TTCACGGCGG GGATGCGGCC |
| | | AACCGTATCT GCGCCTGCTG CGAACTGCAT ATGGATATTC GGCCGCTGCC |
| | | GGGCCTGACG CTAAGCGATC TCGACGGGCT GCTGAATGAG GCGCTGGCTC |
| | | CGGTCAGCGC GCGCTGGCCG GGCCGTCTGA CCGTTGGCGA GCTGCACCCG |
| | | CCCATTCCGG GCTACGAGTG TCCGCGTGAC CACCAGCTGG TGCAGGTGGT |
| | | TGAAAAACTG CTGGGTTGCG AAACCGAGGT GGTGAACTAT TGCACCGAGG |
| | | CGCCGTTTAT TCAGCAAATC TGCCCAACGC TGGTGTTAGG CCCGGGATCG |
| | | ATCGACCAGG CGCATCAGCC GGATGAATTT ATCGATACCG CCTTTATCAA |
| | | GCCAACGCGG GAATTAATTG CTCAGGTGGT GCACCACTTT TGCCATCATT |
| | | AG |
| 7 | Erwinia | ATGCAAGACA AAAGCATCAA GAAAATCGTG CTGGCTTACT CCGGCGGTCT |
| | amylovora | GGATACTTCA GCCATCATTC CGTGGCTGAA AGAGAACTAT GGCGGCTGCG |
| | argG | AAGTGGTGGC GTTCGTGGCG GATATCGGCC AGGAGCGTGG CGACCTGGAA |
| | | GGCGTGGAGC AGAAAGCGCT GCAGTCCGGT GCGTCAGAGT GCCATGTGGT |
| | | GGATCTGCGT GAAGAGTTCA TCAGCGAGTA CGTTTATCCG GTGCTGCAAA |
| | | CCGGTGCCCT GTATGAAGGC ACCTACCTGC TGGGTACGTC GATGGCGCGC |
| | | CCGATTATCG CCAAAGCCCA GGTAGAGCTG GCGCTGAAAG TGGGAGCCGA |
| | | TGCCCTGTGC CACGGTGCAA CCGGCAAAGG GAATGACCAG GTGCGTTTCG |
| | | AAACCACTTA CACCGCGCTG GCGCCACAGC TGAAAGTGGT GGCGCCATGG |
| | | CGTGAATGGG ATCTGCGCTC GCGTGAGGCG CTGCTGGGCT ATCTGAAAGA |
| | | GCGTCATATC CCGACTACCG CATCGCTGGA AAAAATCTAC AGCCGTGACG |
| | | AAAACGCCTG GCATATCTCC ACCGAAGGCG GCGTGCTGGA AAGCCCGTGG |
| | | AACGCCCCGA ACAAAGACTG CTGGGTGTGG ACCGTCGATC CGCTGGAAGC |
| | | GCCGGACCAG CCAGAACAGG TCACCGTTGC CGTTGAGAAA GGTCGCGTCG |
| | | TGGCGGTAAA CGGCGAAGCG TTAAGCCCGT TCGGCTGCCT GGACAAGCTG |
| | | AATGCAATTG GTGCGCGTCA CGGCGTGGGC CGTATCGACA TCGTGGAAAA |
| | | CCGCCTGGTG GGCATTAAGT CGCGCGGCTG CTACGAAACC CCCGGTGGCA |
| | | CCATTATGGT CAACGCGCTG CGTGCGGTTG AACAGCTGGT TCTGGATCGT |
| | | GACAGCTTTA AATGGCGCGA GCAGCTGGGG CTGGAAATGT CCTATGTGGT |
| | | TTACGATGGC CGCTGGTTCG CACCGCTGCG TAAATCAATC CAGGCTTCCG |
| | | CCGAAGCGCT GGCTGAAGAA GTGAACGGTG AAGTGGTGCT GCAACTCTAC |
| | | AAAGGTCAGG TTACCGCCAT TCAGAAGAAA TCAGCCAACA GCCTGTATTC |
| | | CGAAGAGTTT GCCACTTTCG GCGAAGACGA AGTGTACGAT CACCGCCATG |
| | | CAGGCGGCTT TATCCGTCTG TTCTCACTCT CTTCACGCAT CCGCGCGTTG |
| | | AACGAAAAGA AGTAA |
| 8 | Erwinia | ATGGCACTTT GGGGTGGACG GTTTACGCAG CCAGCAGATC AACGTTTCAA |
| | amylovora | GCAGTTTAAT GACTCACTGC GCTTCGACTA TCGTCTGGCA GAGCAGGACA |
| | argH | TTATCGGCTC CGTCGCCTGG TCCAAAGCGC TGGTAACGGT CAATGTATTA |
| | | ACCCACGCGG AGCAGCAGCA GCTGGAAAGC GCGCTGAATG CGCTGCTGGA |
| | | TGAAGTGCGC GCCGATCCGC AACAGATTCT GGCCAGTGAT GCCGAAGATA |
| | | TTCATAGCTG GGTGGAAGGC AAACTGATTG ATAAAGTCGG CGCGCTGGGT |
| | | AAGAAACTTC ACACCGGGCG TAGCCGCAAT GACCAGGTCG CGACCGACCT |
| | | GAAACTGTGG TGCAAAGCGC AGGTCAGCGA GCTGCTGGCC GCCACGCGTC |
| | | AGTTCCAGCA GGCGCTGATC GCCACCGCGG AAGCCAATCA GGATGCGGTG |
| | | ATGCCGGGTT ATACTCACCT GCAACGCGCT CAGCCGGTCA CCTTTGCACA |
| | | CTGGTGCCTG GCCTATGTTG AGATGCTGGC GCGTGATGAA AGCCGGCTGC |
| | | AGGATACGCT GAAACGCCTG GACGTCAGTC CGCTGGGCTG CGGCGCTCTG |
| | | GCGGGAACCG CTTATGAAAT CGATCGTGAG CAGCTGGCCG GCTGGTTAGG |
| | | TTTTGCTTCT GCTACCCGCA ACAGCCTTGA TACCGTATCT GACCGCGACC |
| | | ATGTGCTGGA ACTGCTGTCC GATGCTTCCA TCGGCATGGT GCACCTGTCG |
| | | CGCTTCGCGG AAGATCTGAT TTTCTTCAAT ACCGGTGAAG CCGGGTTTGT |
| | | GGAGCTGTCC GACAAGGTGA CTTCTGGCTC CTCGCTGATG CCGCAGAAGA |
| | | AAAACCCGGA CGCGCTGGAG CTGATCCGTG GCAAGGTTGG CCGCGTACAG |
| | | GGTGCTCTGA CGGCGATGAG CATGACGCTG AAAGGCCTGC CGCTGGCCTA |
| | | TAACAAAGAT ATGCAGGAAG ACAAAGAAGG GCTGTTCGAC GCGCTCGACA |
| | | CCTGGGCTGA CTGCCTGCAT ATGGCGGCGC TGGTGCTGGA TGGCATTCAG |
| | | GTGAAACGTC CGCGCTGTAA GGAAGCGGCC GAGCAGGGCT ATGCCAATTC |
| | | CACCGAACTG GCGGATTATC TGGTGGCTAA AGGCGTGCCC TTCCGTGAAG |
| | | CGCACCATAT AGTTGGCGAA GCGGTAGTGG AAGCGATTAA ACGGGGCGTG |
| | | GCGCTCGAAG CGCTGAAGCT GTCAGAATTG CAGCAGTTCA GTCGCGTCAT |
| | | CGGCGACGAT GTTTATCCGA TTCTGTCACT GCAATCCTGT CTGGATAAGC |
| | | GTAACGCGAA AGGCGGCGTG TCTTTGCATC AGGTATCGCA AGCGATTACC |
| | | GAAGCCAAAC GGCTTCTGGC TTAA |
| 9 | Erwinia | ATGAATCCGT TATATCAACG TCACTTTCTC AGGCTGCTCG ATTTCAGCCC |
| | amylovora | CTCTGAAATC AATGGCTTAC TGGCGCTGGC ATCCCGCCTG AAGTTCGCTA |
| | argI | AGAAAAAGGG CGAAGAGATT CAATATCTGA CAGGCAAAAA CATTGCGCTC |
| | | ATCTTCGAAA AAGACTCGAC TCGTACCCGA TGCTCTTTCG AAGTTGCCGC |
| | | ATTCGATCAG GGCGCTAACG TCACCTATCT TGGCCCAAGC GGCAGTCAGA |
| | | TCGGCCATAA AGAGTCGATT AAAGACACCG CACGCGTGCT TGGCCGCATA |
| | | TATCACGCCA TTCAGTATCG CGGACACGGC CAGCAGCTGG TGGAATCGCT |
| | | GGCCGAACAC GCTGGCGTGC CGGTATGGAA CGGGCTGACC AATGAATTCC |
| | | ACCCTACCCA GCTGCTGGCC GACCTGTTGA CGATGCAGGA GCATTTGCCG |
| | | GGGAAAGCCT TTAGTCAGAT GACGCTGGCC TATGTGGGTG ATACGCAAAA |
| | | CAATATGGGT AACACCCTGC TGGAAGCAGC AGCGCTGGTC GGCCTCGATC |
| | | TGCGCCTGGT GGCACCAAAA AGCTGCTGGC CGGATGCAGC GCTGGTGGCC |
| | | GAATGCCGGG ATGTGGCACA GCACAACGGC GGGAAAATCA CCCTGACGGA |
| | | AGATATTGCC ACAGGGGTAC AAGGCGCTGA TTTTATTTAT ACCGACGTCT |
| | | GGGTTTCGAT GGGCGAACCC AAAGAAGTAT GGCATGAACG CATTGCCCTG |
| | | CTGCGTCCGT ACCAGGTGAA TATGGCGCTG CTGCAGGCGA CGGGAAACCC |
| | | CCGGGTGAAG TTCCTGCACT GCCTGCCCGC CTTCCACGAC GATCAGACCC |
| | | TGCTCGGTCA GCAGATGGCG CAGCAGTACG ATCTGCACGG CGGCATGGAA |
| | | GTGACCGATG AAGTGTTTGA ATCCGCGCAC AGCGTGGTGT TCGATCAGGC |
| | | GGAAAACCGT CTGCATACCA TCAAGGCGGT GATGGTCGCC ACGCTGTCAA |
| | | AGCAGGAGTA A |
| 10 | Erwinia | ATGCTTGAAC AAATGGGCCA GGCCGCGAAG GCCGCCTCTT ATCAGCTGGC |
| | amylovora | GGTGCTCTCC ACCGCAGAGA AAAATCAGTT ACTGATGACG ATTGCCGACC |
| | proA | GTCTGGAAGC GCAGAGCGCA GAGATCCTCG CGGCTAACCA ACTGGATCTT |
| | | GCCGACGCGC GGCAAAACGG CATGAGCGAG GCGCTACAGG ATCGTCTGAT |
| | | GCTGGATCGG CAACGTCTGA AAGGCATCGC CAATGACGTT CGTCAGGTGT |
| | | GCCGCCTGCC CGATCCGGTC GGAGTGGTGA TTGACGGCGG TAAGCTCGAT |
| | | AGCGGACTGC GCATTGAACG CCGTCGCGTG CCGCTCGGCG TGGTCGGGGT |
| | | GATTTATGAG GCGCGCCCGA ACGTTACCGT GGATGTCGCC AGCCTGTGCC |
| | | TGAAAACCGG TAATGCGGTG ATCCTTCGCG GCGGCAAAGA GACCTACCGC |
| | | ACTAATGCTG CCACCGTGCG CGTTATTCAG GATGCGTTGA AACAGTACGG |
| | | CGTGCCGGCT GGTGCGGTGC AGGCGATTGA AAGCCCGGAT CGTGCGTTGG |
| | | TGACTCAGCT GCTGAAGCTC GATCGTTATG TCGATATGCT GATCCCGCGT |
| | | GGTGGTGCCG GACTGCACAA GCTGTGCCGA GAGCAGTCAA CCATACCGGT |
| | | GATCACCGGT GGTATTGGCG TTTGCCATAT TTACGTCGAT CGCAGTATTG |
| | | AGCCGGAAGC GGCGCTAAAG GTGATCGTTA ATGCCAAGAA GCAGCGGCCG |
| | | AGCGCCTGTA ACTCGTTGGA AACCCTGCTG ATTGATGCGG CGATCGCCGA |
| | | CAGTTTCCTG CCAGCGCTCA GCGCGCGCAT GGCGCAGGAG GGGGTTAGCC |
| | | TGCACGCCGA TGTGCGCTCG CTGCCGCTGT TGCAGCAGGG TCCTGCCAGC |
| | | GTCAGCGCGG TTACCGATGC CCAGTACCGC GACGAATGGC TGGCGCTGGA |
| | | TCTGAACGTT AAACTGGTGG ATGACCTCAC TGAAGCGATT GCGCACATCC |
| | | GCGAATACGG CACCCAGCAC TCCGATGCCA TCCTGACACG CACCATTAAG |
| | | CATGCGGAGC GCTTCGTCAA TGAAGTGGAC TCATCTGCGG TTTACGTTAA |
| | | CGCCAGCACC CGTTTCACCG ACGGCGGACA GTTTGGTTTA GGCGCTGAAG |
| | | TGGCGGTCAG TACGCAGAAA CTGCACGCTC GTGGCCCGAT GGGGCTGGAA |
| | | GCGCTGACTA CCTATAAATG GGTGGCGTTC GGTGACGATA CCGTGCGGGG |
| | | TTAA |
| 11 | Erwinia | ATGATGGAAA AGAAAATTGG TTTTATTGGC TGCGGCAACA TGTCCAAAGC |
| | amylovora | CATTATTGCT GGCCTGGTTT CGGCCGGACA GTTTAAAGCA GAAAATATCT |
| | proC | GGGTCTTTGA CCATAAACCC GCCACTAATC AGGCCATGCA GCAGCAGTAC |
| | | GGCATTACTC CGGCAGAAAG CGCCCGCCAG CTGGCGGAAC AGGCGGATAT |
| | | TCTGTTTGGC GCGGTCAAGC CAAACAGGAT CCTGTCCGTG CTGAAGGATG |
| | | TGGCCGGCAG CCTCAACAAA GAGACGCTGG TGGTCTCCAT CGCAGCGGGC |
| | | ATCACCCTGG ATTCACTGGC ATCCGTGCTT GGCCATGACC GTAAAATCGT |
| | | GCGCGTTATG CCAAATACCC CGGCCCTGGT CAACGAAGGC ATGAGTTCGG |
| | | TTACCCCGAA CGTGCTGGTC ACCCAAGAAG AAGCGGATGA AGTGGTAAAC |
| | | ATTTTTAGCG GTTTTGGCAA AGCAGCGCTG GTACCGGAAT ACCTGATCCA |
| | | CGCGGTCGTT GGCGTAAGCG GCTCCGCGCC GGCCTATGTG TTTATGTTTA |
| | | TCGAAGCGAT GGCAGATGCG GCAGTGCTGG GCGGAATGCC TCGTGAGCAG |
| | | GCGTATCAGT TTGCAGCGCA ATCGGTAAAA GGTGCCGCGC AGATGGTACT |
| | | GGAAACCGGT AAACATCCGG CTGAACTCAA GGATATGGTG TGCTCGCCGG |
| | | GCGGTACTAC CATTGAAGCG GTAAAAGTTC TGGAGCAGAA AGGCTTACGT |
| | | TCAGCGGTGA TCGAAGCCAT ACAGCAGTGT ATGAATAAAT CAGAACAGAT |
| | | GAGCAAGCAG TAA |
| 12 | Erwinia | ATGCGAGTGC TGAAATTCGG TGGAACCTCG GTAGCCAATG CGGAACGTTT |
| | amylovora | TCTGCGTGTG GCTGACATTT TGGAAAGCAA TGCACAACAG GGACAGGTTG |
| | thrA | CTACCGTGTT GTCCGCGCCG GCAAAAATTA CCAACCATCT GGTGGCAATG |
| | | ATTGAGAAAA CCATTAGCGG CCAGGATGCT TCAGCGAATA TCAGCGATGC |
| | | AGAGCGTATT TTCGCCGCGC TGTTGCAGGG GCTGACCGAC GCCCAGCCGG |
| | | GGTTTGCTTA TGACCGTCTG AAAGCGGTGG TGGACCAGGA ATTCCTCCAG |
| | | CTCAAACAGG TGCTGCATGG TATTGCGCTG CTGGGGCAGT GCCCGGACAG |
| | | CGTCAATGCG GCCATTATCT GCCGGGGTGA AAAGCTCTCA ATCGCCATAA |
| | | TGGAAGGTGT GTTGCAGGCG CGCGGCCACG GGGTTAGCGT GATTAATCCG |
| | | GTCGAGCAGC TGTTGGCCAT CGGCCATTAT CTGGAATCCA CCGTTGATAT |
| | | TGCCGAATCC ACCCGCCGTA TCGCCGCCCG CCAGATCCCG GCCAGTGATA |
| | | TGGTGCTGAT GGCCGGCTTT ACCGCCGGTA ATGAACATGG AGAACTGGTG |
| | | GTGCTGGGGC GTAATGGCTC AGATTACTCT GCGGCGGTGC TGGCAGCCTG |
| | | TCTGCGCGCT GACTGTTGCG AAATCTGGAC CGATGTTGAC GGCGTGTACA |
| | | CCTGTGACCC GCGTCAGGTA CCGGATGCGC GGCTGCTGAA GTCGATGTCC |
| | | TATCAGGAAG CGATGGAGCT TTCCTATTTC GGCGCTAAAG TTCTTCACCC |
| | | GCGCACTATC GCTCCTATCG CCCAGTTCCA GATCCCCTGT CTGATCAAAA |
| | | ATACCGGCAA TCCGCAGGCC CCCGGAACCC TGATAGGCGG TAACGGCAGC |
| | | AATGATGACA ACCCGGTAAA GGGCATCACT AACCTCAACA ATATGGCGAT |
| | | GTTCAACGTC TCCGGCCCGG GGATGAAGGG CATGGTCGGC ATGGCGGCAC |
| | | GCGTGTTTGC CGCCATGTCA CGCAGCGGAA TTTCGGTAGT GCTGATCACC |
| | | CAATCCTCCT CGGAATACAG CATTAGCTTC TGCGTATCGC AGAGCGATCT |
| | | GCCGCGTGCG CGTCGGGTGC TGGAAGAGGA GTTCTATCTG GAGTTAAAAA |
| | | ACGGCCTGCT GGAACCTTTG GATGTTATGG AGCAGCTGGC GGTGATTTCG |
| | | GTAGTGGGCG ATGGTATGCG CACTCTGCGC GGCATCTCCG CCAAGTTCTT |
| | | TTCAGCGCTG GCGCGGGCCA ACATTAATAT CGTCGCTATC GCCCAGGGAT |
| | | CGTCCGAGCG TTCCATCTCG GTGGTGGTCA ACAATGATCA GGCGATCACC |
| | | GGCGTGCGCG TGGTTCATCA GATGCTGTTT GCCACCGACC AGGTGATCGA |
| | | AGTCTTTGTG ATCGGCGTTG GCGGCGTGGG TGCTGCGCTG ATCGAACAGC |
| | | TGCATCGCCA ACAAATCTGG TTGAAAAATA AACACATCGA TCTGCGTGTC |
| | | TGTGGCATAG CCAACTCGCG GGCGTTGCTG ACTAACGTGC ACGGCATCCC |
| | | GCTGGATAGC TGGCAGGATC GGCTAAAAGC GGCGCAACAA CCGTTTGATC |
| | | TTGGGCGGCT TAATCGCCTG GTGAAAGAGT ACCACCTGCT CAACCCGGTG |
| | | ATTGTCGACT GTACTTCCAG CCAGCAGGTG GCGGATCGGT ACGCTGATTT |
| | | CCTCGCTGAC GGCTTCCATG TGGTGACGCC GAACAAAAAG GCCAATACGT |
| | | CATCGTACAA TTACTATCAG CAAATGCGAA CGGCAGCGGC AAAAGCGCGC |
| | | CGCAAATTCC TCTACGATAC CAATGTGGGT GCCGGTCTGC CGGTTATCGA |
| | | GAACCTGCAA AATCTGCTGA ATGCGGGGGA TGAATTGATC AGCTTCTCCG |
| | | GCATCCTTTC CGGTTCCCTG TCGTTTATTT TCGGCAAGCT GGATGAAGGC |
| | | GTTTCGCTGT CTGATGCCAC CCAAATGGCG CGTGAAATGG GCTTTACCGA |
| | | GCCGGATCCG CGTGACGATT TGTCCGGTAT GGACGTGGCG CGTAAGCTGC |
| | | TGATCCTCGC GCGCGAAGCA GGTTATCAGC TGGAGCTAAG CGATATAGAA |
| | | ATTGAAGCGG TGCTGCCGCC AGAGCTTACC GCCATTGCCG ATGTTGACGA |
| | | GTTTATGGCG CGTTTGCCGG AGCTGAACGA AGGCTTTGCC GCGCGGGTGT |
| | | CCAGCGCCCG TGATGCCGGA AAAGTGTTAC GTTTTGTTGG TGCCATTGAG |
| | | GAAAGCGGGG CGTGCAGGGT TAAAATCGCT GCTGTTGACG GTAACGATCC |
| | | ACTGTATAAA GTGAAAAATG GTGAAAATGC GCTGGCGTTC TATAGCCGTT |
| | | ATTACCAGCC GATCCCACTG GTGCTGCGCG GTTACGGCGC CGGTAATGAT |
| | | GTGACCGCAG CGGGCGTATT TGCCGATCTG TTACGTACAC TGTCGTGGAA |
| | | GTTGGGAGTT TAA |
| 13 | Erwinia | ATGGTTAAAA TTTATGCCCC TGCCTCAATT GGCAATGTCA GCGTCGGTTT |
| | amylovora | CGACGTGCTG GGGGCGGCGG TATCGCCGGT TGATGGCACC CTGCTGGGCG |
| | thrB | ACTGCGTCAG CGTGGAAGCC GCCACTGAGT TCAGCCTGCA TAATGAAGGT |
| | | CGTTTTGTCA GCAAGCTGCC CGCTGACCCA AAAGAAAATA TTGTTTACCA |
| | | GTGTTGGCAG CGTTTCTGCA CCGCCATTGG CCAGCACGTC CCGGTTGCCA |
| | | TCACGCTGGA AAAAAACATG CCGATTGGCT CCGGCCTTGG CTCCAGCGCC |
| | | TGCTCGGTGG TTGCCGGGCT AATGGCGATG AACGAATACT GCAACAAGCC |
| | | ATTAAATGAT AACGAGCTGC TGGTATTAAT GGGCGAGCTG GAAGGGCGCG |
| | | TCTCCGGCAG CGTTCACTTT GACAACGTGG CACCCTGTTT TCTTGGCGGT |
| | | ATGCAGCTCA TGCTGGAGGA AAACGGCATT ATCAGCCAGC CGGTGCCAGG |
| | | CTTTGATGAC TGGCTGTGGG TGATGGCCTA TCCGGGCATC AAGGTGTCGA |
| | | CGGCGCAAGC GCGTGCCATC TTGCCCGCGC AGTACCGCAA AGAAGATATT |
| | | ATTCGCCACG GTCGCTATCT GGGCGGCTTT ATTCACGCCT GCCATACACA |
| | | GCAGCCGCTG CTGGCCGCGA AGCTGATGCA GGATGTAGTT GCTGAACCTT |
| | | ATCGCACCAA ACTGTTACCG GGCTTTGCCC AGGCTCGCCA GGCAGCTACT |
| | | GATATTGGCG CGCTGGCATG CGGTATTTCC GGCTCCGGGC CAACGCTGTT |
| | | TGCCGTTTGT AATCAGCCGG ATACCGCAAA ACGTATGGCA GACTGGCTGG |
| | | GTCAGCACTA TCTGCAAAAT GAGGAAGGCT TCGTTCATAT CTGCCGTTTA |
| | | GACACGGCAG GCGCACGTAA ACTGGGATAA |
| 14 | Erwinia | ATGACACGTT TAAATCCTTA TTTTGGTGAG TTTGGCGGCA TGTATGTGCC |
| | amylovora | GCAGATCCTG ATGCCTGCCC TGCTGCAGCT GGAAGATGCT TTTATCAGCG |
| | trpB | CGCAAAGCGA TCCGGCATTT CAGGCTGAAT TTACCGACCT GCTGAAAAAT |
| | | TATGCTGGCC GTCCAACCGC GCTGACACTG TGTAAAAACC TGACCAAAGG |
| | | CAGCAAAACG CGCCTTTATC TGAAGCGTGA AGATCTGCTG CACGGCGGCG |
| | | CGCACAAAAC CAATCAGGTG CTGGGCCAGG CACTGTTGGC CAAGCGCATG |
| | | GGCAAAAACG AAATTATTGC TGAGACGGGT GCCGGTCAGC ATGGCGTTGC |
| | | CTCGGCGCTG GCCTGCGCGC TGCTTGGCCT GAAATGCCGT ATCTATATGG |
| | | GTGCGAAAGA TATAGAGCGC CAGTCGCCTA ACGTATTTCG TATGCGCCTG |
| | | ATGGGTGCGG AAGTGATCCC GGTGCACAGC GGTGCTGCCA CGTTGAAAGA |
| | | TGCCTGTAAC GAGGCGCTGC GCGACTGGTC CGGCAGCTAC GAAACCACTC |
| | | ACTATATGCT GGGTACGGCG GCTGGCCCTC ATCCGTTCCC GACCATCGTA |
| | | CGCGAATTCC AGCGCATGAT TGGTGAAGAG ACTAAAAAGC AGATCCTGCA |
| | | ACAGGAAGGC CGCCTGCCGG ATGCGGTCAT CGCCTGTGTC GGCGGGGGTT |
| | | CTAACGCCAT CGGCATGTTT GCTGACTTTA TCGATGAATC CGGCGTGGAG |
| | | CTGATTGGCG TGGAACCGGG CGGTCATGGT ATTGAAAGCG GCGAACATGG |
| | | CGCTCCACTC AAGCACGGGC GAGTCGGTAT TTATTTTGGC ATGAAATCAC |
| | | CGATGATGCA GACATCTGAC GGGCAGATTG AAGAATCTTA CTCGATTTCA |
| | | GCCGGTCTCG ACTTCCCTTC CGTCGGCCCG CAGCATGCTC ACCTCAACAG |
| | | TATCGGCCGG GCGAACTACG TATCAATTAC CGATGACGAA GCGCTGGATG |
| | | CCTTTAAAGC GCTGTGCCGC AGCGAGGGGA TTATCCCGGC GCTGGAATCC |
| | | TCACATGCGC TGGCCCATGC GCTGAAAATG GCGCGCGAAA ATCCTGAAAA |
| | | AGAGCAGCTG TTAGTGGTCA ATCTTTCCGG CCGTGGTGAC AAAGACATCT |
| | | TCACCGTGCA CGACATCCTT ACCGCTAAGG GAGAAATCTG A |
| 15 | Erwinia | ATGCAGCAAA CCGTACTTGA TCGCATCGTG CGCGATAAAA CCGACTGGCT |
| | amylovora | GGCGGCTCGC CAGCAGCAGC AGCCTCTGGC CTCGTTTCAG CATGAGATTG |
| | trpCF | TCCCGGCTAC GCGCAACTTC TACGAGGGGC TGAAGGGCGC CCGTACCGCG |
| | | TTCATTCTGG AGTGCAAAAA AGCCTCGCCG TCCAAAGGAT TGATCCGTGA |
| | | AGACTTTGAC CCGGCGGTGA TTGCCGGCGT CTACCGGCAT TACGCCTCCG |
| | | CCATCTCGGT GCTGACCGAT GAGAAGTACT TTCAGGGAAG CTTTGACTTT |
| | | CTCCCCATCG TCAGTGCAGC AGTCAGTCAA CCGGTACTGT GTAAAGACTT |
| | | TATCATCGAC CCGTATCAGA TCCATCTGGC GCGCTATTAT CAGGCCGACG |
| | | CCATTCTGCT GATGCTGTCG GTACTGAATG ACGAGCAGTA CCGCCAGCTG |
| | | GCCGCCGTGG CACACAGCCT GAATATGGGC GTGCTGACGG AAGCGATCAG |
| | | CGATGAAGAG CTTGAGCGCG CCACCGCGCT GGGCGCAAAA GTGGTCGGCA |
| | | TCAATAACCG TGATTTACGC GATCTGTCAA TCGACCTCGA CCGTACCCGC |
| | | CGTCTGGCGC CGAAAGTGGC ACAGGGCGTG ACGGTCATCA GTGAATCAGG |
| | | CATCAACAGC TATGCCCAGG TGCGCGAACT GAGTCACTTC GCCGACGGCT |
| | | TCCTGATAGG TTCGGCGCTG ATGTCGGAAG ACAACCTTGC CGCTGCGGTC |
| | | AGGCGGGTGA TTATCGGTGA GAACAAAGTC TGCGGACTGA CCCGCCCGCA |
| | | AGATGCGCGC AGCGCGCTGG AGTGCGGGGC TATCTATGGC GGGCTGATCT |
| | | TTGCCGCCGG ATCGCCGCGC CAGATTGACG CCGCACGGGC GCAAGAGGTC |
| | | ATGGCCGCCG CCGCACTCAG GTACGTTGGC GTGTTCCGTA ATGACGGCCT |
| | | GAGTGACGTT GTCACTATCG CGCGGCGGCT CAACCTGCAC GCGGTACAGC |
| | | TGCACGGTGA TGAAGATCGC GAGTTCATTA GCCAGCTACG AGCCGCATTG |
| | | CCTGAAAACG TGCAGATCTG GAAAGCCTTC AGCATCAGCA CCAGCCTGCC |
| | | CGCTCGCGAC TGGCCGCACG TCGATCGCTA CGTATTTGAC CAGGGAAATG |
| | | GCGGCAGCGG TAAAGCGTTT GACTGGTCGC TGTTACAAGG TGAAAAGTGG |
| | | GATAACGTCC TGCTGGCCGG CGGCCTGAGT GCGGACAACT GCCTTCTGGC |
| | | CGCACAGCAG GGCTGTGCCG GTCTCGACTT TAATTCCGGC GTGGAGAGCG |
| | | CTCCCGGAAT CAAAGATGCA GCAAAAATGG CTTCCGTGTT CCATACGTTG |
| | | AAAGCCTACT GA |
| 16 | Erwinia | ATGCCAAATG CCAAACCTGG AGTGAAATTG ATCACCGCCA GTGCGCCGTA |
| | amylovora | CCGGGAAGAT CCCGCTGCGG TTTTCCATCA GCTCTGCGCG GCTCGTTCAG |
| | trpE | AAACGCTGCT ACTGGAATCT GCCGACGTCG ACAGTAAATG CAATCTGAAA |
| | | AGCCTGCTGA TTGTTGACAG CGCTCTGCGC ATCAGCGCAC TGGACAATCG |
| | | TGTGCAGATC CAGGCACTGT CGGCCAATGG CCGTGCACTG TTGCCATTGC |
| | | TGGATGCCGC ATTGCCCTTA TCCGTGGTCA ACCATGTGGG TACTGATAGC |
| | | CGTGAGCTGG TGTTTCCGCA CAATGAGGAG ATTCAGGACG AAGACGCTAA |
| | | ACTGCGCGCC CTGTCAGTAT TTGATGCGCT GCGCCTGATC CCTCAACTGG |
| | | TAAATTCTCC GCACCAGGAG CGTGAAGCAA TGCTGCTTGG TGGGCTGTTC |
| | | GCCTATGACC TGGTCGCCGG CTTCGAAACG CTGCCGGAGA TTGCCAACCA |
| | | GCAGCGCTGC CCGGACTACT GCTTCTATCT GGCTGAAACG CTGCTGGTTA |
| | | TCGACCACCA GGCACGGAGC GCTCGCCTGC AGGCCAGCCT GTTTACCCCG |
| | | AACGCGTCTG AGTTCCAGCG TTTGCAGAGC CGCGTAGAAC AGCTGTGCGG |
| | | CATGATGTCT CAACCCGTCA GCGCCTTACC GGTGCAAACG GTGGACAGTA |
| | | TGCAGCTTAG CGTCAGTCAG AGCGATGAAG AATACTGCGC CGTTGTGCGC |
| | | CAGATGCAGG AAGCGATCCG CGTAGGTGAA ATTTTCCAGG TTGTGCCTTC |
| | | GCGCCGTTTC TCTTTGCCTT GCCCTTCCCC GCTGGCCGCT TACGACACGC |
| | | TGAAAAACAG TAACCCCAGC CCGTATATGT TCTTTATGCA GGATCGCGAC |
| | | TTCTGCCTGT TCGGCGCCTC GCCGGAAAGT TCGCTGAAGT ACGAAGCCGC |
| | | CAGCAATCAA ATCGAAATCT ATCCGATTGC CGGAACGCGC CCGCGTGGCC |
| | | GTCACGCCGA TGGCTCGCTC GATCGCGATC TCGACAGCCG TATTGAGCTT |
| | | GAAATGCGTA CCGATCATAA AGAGATGGCC GAACATCTGA TGCTGGTCGA |
| | | TCTGGCGCGC AACGATTTAG CACGTGTCTG CCAGCCCGGT ACTCGCTATG |
| | | TTGCCGACCT GACCAAAGTC GATCGCTACA CCTTCGTTAT GCACCTGGTC |
| | | TCACGCGTTG TCGGTCAGCT GCGTAATGAA ATAGATGTGC TGCACGCGTA |
| | | TCGCGCCTGT ATGAACATGG GTACGCTGAG CGGTGCGCCG AAAGTGCGCG |
| | | CCATGCAGCT GATTGCCAAA GCCGAAGGCA CACGGCGCGG CAGCTATGGC |
| | | GGCGCGGTCG GTTACTTTAC CGCCAGCGGC GATCTCGACA CCTGTATTGT |
| | | TATCCGTTCG GCTTACGTTG AAGACGGTAT GGCAACAGTA CAGGCCGGCG |
| | | CCGGGGTGGT ACTGGACTCC AACCCGCAAG CGGAAGCGGA TGAAAGCCGC |
| | | AATAAAGCCC GCGCGGTTCT GCGCGCCATC GCCTCTGCTC ATCACTGCCA |
| | | GGAGATTTTC TGA |
| 17 | Erwinia | ATGGCCGATA TTCTGTTGCT TGATAACTTT GACTCATTCA CTTACAACCT |
| | amylovora | CGTCGACCAG CTGCGTACCT TTGGCCACCA GGTGACGATT TACCGCAATA |
| | trpG | ACGTCCCGGC AGAGACGCTG ATTGCTTGTC TGCAACAGAT GGAAAGTCCG |
| | | GTGTTGATGC TGTCGCCCGG TCCCGGCGCC CCTGCAGACG CGGGCTGTAT |
| | | GCCAGAACTG CTCCGGCAGC TACGAGGCCG CCTGCCGATC ATTGGTATCT |
| | | GTCTGGGGCA CCAGGCGATA GTGGAGGCAT ACGGCGGCCA CGTCGGTCAG |
| | | GCGGGGGAAA TCCTGCATGG CAAGGCCTCC GCCATCAGTC ATGACGATCA |
| | | CGGCATGTTT GCCGGGCTGA GCAACCCGCT GCCGGTGGCG CGCTACCACT |
| | | CACTGGTTGG CAGCGCGATC CCCGCCGGAC TGACCGTTAA TGCCACTTTT |
| | | AACGATATGG TGATGGCGGT GCGCGATGAC GCTAATCGCG TCTGCGGCTT |
| | | CCAGTTCCAT CCGGAATCCA TTCTGACCAG CCAGGGCGCG CGTCTGCTGG |
| | | AGCAAACCCT CGCCTGGGCG CTGGCGTCAT CAAGGAGCCA TTGA |
| 18 | Erwinia | ATGCAAAAAA AAGCTGAGTT GTATCGCGGC AAAGCGAAAA CCGTTTACAG |
| | amylovora | CACCGAAAAC CCGGATCTGT TGGTACTCGA ATTCCGTAAC GATACGTCAG |
| | purC | CGCTGGACGG TGAGCGTATC GAGCAGTTTG ATCGTAAAGG AATGATTAAC |
| | | AACAAGTTCA ATCATTTCAT TATGACTAAA CTGCAGGAAG CCGGGATCCC |
| | | AACACAGATG GAGGCCCTGC TTTCTGATAA CGAAGCGCTG GTAAAAAATC |
| | | TGGATATGGT GCCCGTAGAA TGCGTAATAC GTAATCGTGC TGCGGGATCC |
| | | CTGGTGAAGC GTCTGGGCGT GGAAGAGGGC ATGATCCTCA ATCCTCCGCT |
| | | GTTTGATCTG TTCCTGAAAG ATGATGCTAA ACACGATCCG ATGGTCAACG |
| | | AATCCTATTG CGAGACGTTC GGCTGGGTCA GCAAAGCAAA TCTGGCACGT |
| | | ATGCAGGAGC TGACTTTCCA GGCGAATGAC GTCCTGAGCA AGCTGTTTGC |
| | | CGATGCAGGC CTGATCCTGG TGGACTTCAA ACTTGAGTTC GGCCTGTTTA |
| | | AAGGCGAAGT GGTGCTGGGC GATGAGTTCT CACCGGATGG TGCGCGCCTG |
| | | TGGGATAAAG ACACCCTGAA TAAGATGGAC AAAGATCGTT ATCGTCAGAG |
| | | CCTTGGTGGG CTGATTGAAG CCTATGAAGA GGTGGCCAAC CGGTTGGGCG |
| | | TGAAGCTGGA CTAA |
| 19 | Erwinia | ATGGCCAATC CGCTATATCG CAAACATATT ATTTCCATTA ACGATCTCAA |
| | amylovora | CCGTACTGAG CTGGAGCTGG TGCTGAACAC CGCCGCCAGC CTGAAAGCGG |
| | pyrB | CCCCGCAGCC GGAACTGTTG AAGCATAAAG TGATTGCCAG CTGCTTCTTT |
| | | GAAGCCTCAA CCCGCACCCG CCTGTCGTTT GAAACCGCCA TGCACCGCCT |
| | | CGGGGCGTCG GTGGTGGGCT TTGCCGACGG CGGTAATACC TCTCTGGGGA |
| | | AGAAAGGCGA AACCCTGGCC GATACTATTT CGGTGATTAG CACCTATGTG |
| | | GATGCCATCG TGATGCGCCA TCCACAAGAA GGGGCCGCCC GACTGGCAAC |
| | | CGAGTTTTCC GGCGGCGTGC CGGTACTGAA CGCGGGGGAC GGAGCCAACC |
| | | AGCATCCAAC CCAGACGCTG CTCGACCTGT TTACCATTCA GGAAACCCAG |
| | | GGGCGTTTGA GCCATTTGCA GGTCGCGATG GTTGGCGACC TGAAATATGG |
| | | TCGTACCGTG CACTCGCTGG CTCAGGCGCT GGCGAAATTT GACGGCAACC |
| | | GCTTCTGGTT TATCGCCCCG GATGCGTTGG CGATGCCGGC TTATATCACC |
| | | GATATGCTGG ACGAAAAAGG CATTGCCTGG ACGCGCCACG ACAGCATTGA |
| | | GGAGGTAGTG CCACAGGTGG ATATTCTGTA TATGACGCGC GTGCAGAAAG |
| | | AGCGGCTGGA TCCCTCGGAA TATGCCAATG TCAAAGCGCA GTTTGTGCTG |
| | | CGCGCTGCGG ATTTGAACGG TGCGCGTGAC AATATGAAGG TGCTGCATCC |
| | | GCTGCCGCGT ATTGATGAAA TCGACAACGA CGTCGACAGC ACGCCGCACG |
| | | CCTGGTATTT CCAGCAGGCT GGCAACGGCA TTTACGCACG CCAGGCGCTG |
| | | CTGGCGCTGG TACTCAACAG TGAACCGGCT CTGTAA |
| 20 | Erwinia | ATGCTAAGAA TCACTAAAGA AGCCCTGACA TTCGACGACG TTCTGCTCGT |
| | amylovora | TCCTGCTCAC TCCACGGTGC TGCCAAACAC CGCCGACCTC AGTACCCAGC |
| | guaB | TCACTAAAAA CATCCGTCTG AACATTCCTA TGCTGTCTGC TGCTATGGAC |
| | | ACCGTGACCG AAGCCGGTCT GGCCATTGCG CTGGCACAGG AAGGGGGCCT |
| | | GGGCTTTATC CACAAAAACA TGTCTATTGA GCGCCAGGCG GAAGAAGTTC |
| | | GCCGGGTGAA GAAGCATGAA AGCGGCGTGG TTACCGACCC GCAGACCGTG |
| | | TTGCCCACTA CCGCGCTTAG CGAAGTGAAA GCATTGACCG AACGTAACGG |
| | | CTTTGCCGGT TACCCGGTGG TCAACGGCGA AAACGAGTTG GTCGGTATTA |
| | | TTACCGGACG TGATGTCCGC TTTGTCACCG ATCTGAGCCT GCCGGTTTCT |
| | | GCGGTAATGA CGCCAAAAGA GCGTCTGGTC ACGGTGAAAG AGGGGGAAGC |
| | | GCGCGAAGTG GTGCTGCACA AAATGCACGA AAAACGCGTG GAAAAAGCGC |
| | | TGGTGGTGGA TGACAGTTTC CATCTGCTGG GCATGATCAC CGTGAAAGAC |
| | | TTCCAGAAAG CGGAACGTAA ACCCCTTGCC TGTAAAGACG AGCATGGCCG |
| | | TCTGCGCGTG GGTGCTGCCG TCGGTGCAGG TGCCGGTAAC GAAGAGCGCG |
| | | TCGATGCGCT GGTGGCTGCC GGTGTTGACG TACTGCTGAT CGATTCCTCC |
| | | CACGGTCATT CTGAAGGCGT TTTGCAACGC ATCCGCGAAA CACGTGCTAA |
| | | ATACCCCGAT CTGCAAATTG TCGGCGGCAA CGTGGCGACC GGCGCTGGCG |
| | | CGCTGGCGCT GGCCGAAGCC GGCGTCAGCG CGGTGAAAGT CGGTATTGGT |
| | | CCTGGCTCAA TCTGTACTAC CCGCATCGTG ACCGGCGTGG GCGTACCGCA |
| | | GATCACCGCA GTTTCTGACG CCGTTGCCGC GCTGGAAGGC ACCGGCATTC |
| | | CGGTTATCGC TGACGGCGGC ATCCGCTTCT CCGGCGATAT CGCAAAAGCT |
| | | ATCGCCGCCG GTGCTGCTGC GGTGATGGTT GGCTCCATGC TGGCAGGTAC |
| | | TGACGAGTCG CCGGGCGAAA TCGAACTGTA CCAGGGGCGT TCATACAAGT |
| | | CTTACCGTGG CATGGGCTCG CTGGGCGCGA TGTCGAAAGG TTCTTCCGAC |
| | | CGTTACTTCC AGACCGATAA TGCGGCGGAC AAACTGGTGC CGGAAGGCAT |
| | | CGAAGGTCGC GTGGCATACA AAGGTCGCCT GAAAGAGATC GTGCATCAGC |
| | | AAATGGGCGG GCTGCGCTCC TGTATGGGCC TGACCGGCTG TGCTACTATC |
| | | GACGAGCTGC GCACCAAAGC GCAATTCGTG CGCATCAGCG GTGCCGGTAT |
| | | TTCGGAAAGT CACGTTCACG ATGTGACCAT TACTAAAGAG TCGCCGAACT |
| | | ACCGTATGGG TTCCTGA |
| 21 | Erwinia | ATGACTATCA AAGTAGGTAT CAACGGTTTT GGCCGTATCG GTCGCATCGT |
| | amylovora | TTTCCGTGCT GCTCAGGAAC GTTCTGACGT AGAAATCGTT GCTATCAACG |
| | gap A | ATCTGCTCGA CGCAGATTAC ATGGCTTACA TGCTGAAGTA TGACTCAACT |
| | | CATGGTCGCT TTAACGGCAC TGTAGAAGTC AAAGACGGCC ATTTGGTTGT |
| | | TAACGGCAAA ACCATCCGCG TTACTGCTGA GCGCGATCCG GCGAACCTGA |
| | | AGTGGGATGC GGTTGGCGTT GATGTGGTTG CTGAAGCAAC CGGTATCTTC |
| | | CTGACCGATG AAACTGCTCG TAAACACATC GAAGCTGGCG CTAAGAAAGT |
| | | GGTACTGACT GGTCCGTCCA AGGATGATAC CCCGATGTTC GTAATGGGGG |
| | | TTAACCACAA GGCTTACGCG GGTCAGGCTA TCGTTTCTAA CGCATCCTGC |
| | | ACCACCAACT GCCTGGCACC GCTGGCGAAA GTGATCAACG ATAAATTCGG |
| | | TATCGTTGAA GCATTGATGA CCACGGTACA CGCCACTACG GCGACTCAGA |
| | | AAACCGTTGA CGGCCCGTCT CACAAAGACT GGCGCGGCGG CCGTGGCGCA |
| | | TCTCAGAACA TTATCCCTTC TTCTACCGGT GCGGCTAAAG CGGTTGGAAA |
| | | AGTGATCCCT GAGTTGAACG GCAAGCTGAC CGGTATGGCG TTCCGCGTTC |
| | | CTACACCTAA CGTTTCCGTT GTTGACCTGA CTGCACGTCT GGAAAAGCCT |
| | | GCTTCTTATA AAGAAATTTG TGACACGATT AAAGCGGCTG CCGAAGGTGA |
| | | ATTGAAAGGC ATTCTGGGCT ACACCGAAGA TGAAGTGGTT TCTACCGATT |
| | | TTAACGGCGA AAAGTTGACC TCTATCTTTG ATGCAAAAGC CGGTATCGCC |
| | | CTGAACGATA ACTTTGTGAA ACTGGTATCC TGGTACGACA ACGAAACAGG |
| | | TTACTCGAAC AAGGTGCTGG ACCTGATTGC TCACATCGCT AAATAA |
| 22 | Erwinia | ATGATTTCAG GAATTTTAGC ATCACCGGGT ATCGCTTTCG GCAAGGCGCT |
| | amylovora | TCTGCTGAAA GAAGATGAGA TTGTTATTGA CCGGAAAAAG ATTTCTGCCG |
| | ptsI | ACCAGGTCGA TCAGGAAGTT CAGCGTTTTC TTGAGGGTCG TAAAAAAGCT |
| | | GCCGCACAGC TGGAAACGAT TAAAACCAAA GCAGGTGAAA CCTTTGGCGA |
| | | AGAAAAAGAA GCGATCTTCG AAGGGCACAT CATGCTACTC GAAGATGAAG |
| | | AACTTGAGCA GGAAATCATC GCTCTGATTA AAGATGAGCA CGCCACCGCT |
| | | GATTCTGCTG CCTATTCTGT TATCGATGGC CAGGCCAAAG CGCTGGAAGA |
| | | GCTGGATGAC GAGTATTTGA AAGAGCGTGC TGCCGACGTC CGTGACATCG |
| | | GTAAGCGCCT GCTGCAAAAT ATTCTTGGCC TGCATATTGT CGATCTTAGC |
| | | GCCATCAATG AAGAAGTTCT GCTGATTGCC AAAGATTTGA CTCCGTCTGA |
| | | AACGGCTCAG CTCAACCTGA AAAAAGTATT AGGTTTCATT ACCGATATTG |
| | | GCGGCCGTAC TTCTCACACC TCGATCATGG CACGCTCGCT GGAGCTGCCA |
| | | GCCATTGTTG GTACCGGTGA CGTGACCAGC CGGGTGAAAA ACGACGATTT |
| | | CCTGATCCTC GACGGCGTAA ATAACCAGAT ATACATCAAC CCGACGCCGG |
| | | AAAAAATCGA AGAATTAAAA GCCGTCGCGC ATCAGTACGT CAGCGAGAAG |
| | | AACGAGCTGG CAAAACTGAA GGACCTGCCG GCAATCACCC TTGATGGTCA |
| | | TCAGGTTGAA GTGTGCGCAA ACATCGGCAC CGTACGCGAC GTCGCGGGGG |
| | | CTGAACGTAA CGGCGCTGAA GGCGTTGGCC TGTACCGCAC CGAGTTCCTC |
| | | TTTATGGACC GCGAATCGCT ACCCAGTGAA GATGAGCAGT TCCAGGCCTA |
| | | TAAAGCCGTG GCGGAAGCGG TTGGCGCTCA GGCCGTCATC GTGCGAACTA |
| | | TGGACATTGG CGGTGATAAA GATCTGCCGT ATATGAACCT GCCAAAAGAA |
| | | GACAACCCGT TCCTTGGCTG GCGCGCTATT CGCATCGCGA TGGATCGTCC |
| | | TGAAATCCTG CATGCACAGC TGCGTGCTAT CCTGCGCGCC TCTGCCTTTG |
| | | GCAAGCTGCG CATCATGTTC CCGATGGTGA TTTCCGTTGA GGAAGTTCGC |
| | | ATCCTGAAAG CAGAGCTGGA AACCCTGAAA GCCCAACTGC GTGCGGAAGG |
| | | TAAAGCATTC GACGAGAAAA TCGAAGTGGG TATCATGGTG GAAACGCCTG |
| | | CTTCCGCAGC GATTGCTCAT CATCTGGCGA AAGAAGTCGA CTTTTTCAGT |
| | | ATTGGGACTA ACGATCTGAC TCAGTACACT TTGGCAGTAG ATCGTGGTAA |
| | | TGATCTGATT TCGCATCTCT ACAATCCTAT GTCCCCTTCT GTATTAACGT |
| | | TGATTAAGCA AGTTATTGAT GCTTCTCACG CTGCAGGTAA ATGGACCGGA |
| | | ATGTGTGGTG AGCTGGCAGG TGACGAACGT GCTACACTAC TGTTACTGGG |
| | | AATGGGGCTG GACGAATTTA GCATGAGTGC CATTTCTATC CCACGCATCA |
| | | AGAAAATTAT TCGTAATACC CATTTTGAAG ATGCGAAGGC GTTAGCAGAT |
| | | CAAGCTCTGG CCCAGCCGAC AGTGGATGAA GTGATGAACC TGGTGAATAA |
| | | GTTCATTGAA GAAAAAACAC TCTGCTGA |
| 23 | Erwinia | ATGTCAGCTG AACACGTTCT GTCGATGATG AACGAGCACG AAGTCAAATT |
| | amylovora | CGTTGACCTG CGTTTCACCG ATACCAAAGG CAAAGAGCAG CACGTCACTA |
| | glnA | TCCCTGCTCA CCAGGTTAAT GCTGACTTCT TCGAGGAAGG CAAAATGTTC |
| | | GACGGCTCCT CGATTGGTGG CTGGAAAGGG ATTAACGAAT CCGACATGGT |
| | | TCTGATGCCG GATGCCACCA CCGCTGTCCT CGATCCGTTC TTCGAAGATG |
| | | CCACCCTGAT TATTCGTTGC GACATCCTTG AGCCAGGCAC CATGCAGGGT |
| | | TACGACCGTG ACCCACGCTC CATCGCCAAA CGCGCAGAAG ATTTCCTGCG |
| | | CTCTTCCGGC ATTGCCGACA CCGTGCTGTT CGGGCCAGAA CCCGAGTTCT |
| | | TCCTGTTTGA CGATATCCGT TTCGGCAGCA GCACTTCCGG CTCCCACGTT |
| | | GCTATCGATG ATATCGAAGC CAGCTGGAAC ACCGGTAAAC AATACGAAGG |
| | | CGGCAACAAA GGCCATCGTC CGGGGCTGAA AGGCGGCTAC TTCCCGGTTC |
| | | CTCCGGTCGA CTCATCACAG GATATCCGTT CTGCGATGTG TCTGACGATG |
| | | GAACAGATGG GCCTGGTGGT TGAAGCTCAT CACCACGAAG TCGCCACTGC |
| | | CGGTCAGAAT GAAGTGGCTA CCCGCTTCAA CACCATGACC AAGAAAGCTG |
| | | ACGAAATTCA GATCTACAAA TATGTCGTCC ACAACGTGGC TCACGCTTAT |
| | | GGCAAAACCG CCACCTTTAT GCCTAAGCCA ATATTCGGTG ATAACGGCTC |
| | | CGGTATGCAC TGCCACATGT CACTGTCAAA AGGCGGAACT AACCTGTTTG |
| | | CGGGCGACAA ATACGGCGGC CTGTCTGAAA CTGCCCTGTT CTACATCGGC |
| | | GGAGTGATTA AACACGCTAA AGCGATCAAT GCGCTGGCTA ACCCGACCAC |
| | | TAACTCCTAT AAGCGTCTGG TTCCTGGCTA CGAAGCACCG GTTATGCTGG |
| | | CTTACTCTGC GCGTAACCGT TCCGCGTCTA TCCGTATTCC GGTTGTCGCC |
| | | AGCCCGAAAG CACGCCGTAT TGAAGCGCGC TTCCCCGATC CGGCTGCTAA |
| | | CCCGTACCTG TGCTTCGCGG CACTGTTGAT GGCTGGCCTT GACGGCATCA |
| | | TCAATAAGAT CCACCCGGGC GATGCGATGG ACAAAAACCT GTACGACCTG |
| | | CCGCCGGAAG AAGAAGCGGA GATCCCTAAA GTTGCTGCCT CCCTGGACGA |
| | | AGCTCTGGCC GCTCTCAACG AAGACCGTGA GTTCCTGACG CGTGGCGGTG |
| | | TGTTCACCGA CGACGCGATC GAAGCTTATA TCGAGCTGCG TAAAGCGGAG |
| | | ATGGAGCGCG TGCGCATGAC CCCGCACCCG GTTGAGTTCG AACTGTACTA |
| | | CAGCGTTTAA |
| 24 | Erwinia | ATGTCCAACT CAACACCTTT TCCTTACGGT TTGTACGATC CCTCAAAAGA |
| | amylovora | CAGCGACAGC TGCGGCGTGG GCTTCATTAC GCGTAAGGAT GGCGCGCAAA |
| | gltB | CTCATGAGGT GCTGGAAATG GCACACGGCG CTCTGTGCAC CGTTCCGCAT |
| | | CGTGGGGGAA TGTCAGCCGA AGGCGTGGGT GATGGCGCCG GGGTTAACGT |
| | | AGACCTTTCA CTGCACTTCT TCCGTAAAAT CACCGGCCAG CCGCTGGAAG |
| | | CTGGCCGTTT CGGCGTCGGT AACTTCTTTG TGCCGAAAGA CAGCACGCTG |
| | | CGCGCCAACG CCGAGCGGCT GGTTGAAGAC ACGCTGCTTT CCTGTGACCT |
| | | GAAAATTATT GCCAGGCGTG ATATCCCGCT GGACAACAGC GTGTTGCGCC |
| | | CGGCAGCGAT CCCTTTCCAG CTGCCTGTTG CGCAATGGAT ATTTGCTGCC |
| | | CCGGACGATG TCAGCAACCA GATTGATTTC GAGAAGCGCA TTTATCGCGC |
| | | CCTGCTGCAA ATCGAAGCCC GTGCTTTTAG CGACGGCGAA TTCGGCGGGC |
| | | TTTACCCGCT GTCGCTCTCC TCGCGCACCC AGGTGTTAAA AGCACGCCTG |
| | | AACTCGAATG AAGTGATCCC CTATTTCAAA GATCTGACCG ATCCCGATCA |
| | | TCAGGTACGC GGTCTGTTCT TCCATACGCG CTTCTCAACC AATACCGATC |
| | | CGCATACCAC CATGGCGCAG CCGTTCCGGC TGATGGCGCA TAACGGCGAG |
| | | CTAAACACCG ACCGTAAAAA CCGCATTGCG GAAGCCGCGC TGGCGCTGGC |
| | | GCGCGGTAAG AAAATCGTGC GCCCGAAAGG GCAATCGGAC AGTTCACGTC |
| | | TTGACCAGAG CATCCACAGC CGCCTGATGG AAGACAATCT CGACCTGGTC |
| | | ACCGCCGTGG TTTCCATGAT GCCGCCCGCC TGGGAAAACG ATCCGTCACT |
| | | GTCGCCCGAA GTACGTGCCA TGCTGGAGTA TTTCTCGCTG TATGAAGAGA |
| | | AGAACGACGG CCCGGCCGCG CTGATTTTTG GCAATGGCGA AGTGATCGGC |
| | | GCACGTCTCG ACCGTCTTGG TCTGCGTCCA CTGCGCTCGG TGGAAACCGC |
| | | CGAATATATC GGTGCCATGT CGGAAGCTGG CCAGATCGCC TTCCCGCCGG |
| | | AAAGCGTGCT GCGCCGTGGC CGTATCGAAG CAGGCGGCAT GCTGTATTAC |
| | | GATCACCGCG AAAAGCGCAG TTACACCACC ATTGACGCGC TGGAAAAACT |
| | | GGCGGCGGCG GCTGACTATC CGGCCTTGCT GGCGAAGTCC CGCGTGCTGT |
| | | TGCAGGATCT GCCGGTGGTT CCGGCTGAAC AGCAGGGATC TCCGTTGCGT |
| | | TATGGCGGCG ACCTCAAAAC CTGGCAGCGT TTTGTCGCCT ATTACTATAA |
| | | CCAGGAAAGC TTCAAATTTA TGATGGACCC GATGCTGACC ACCGGCGCAG |
| | | AGAAAATCTC CGCTATGGGT TACGGCAACG CCATCAACGC CCTGTCCGAC |
| | | CATGAAGGCG GCATGGCGCA CTATTTCTCC CAGCGCTTCG CACAGGTTAC |
| | | CAACCCGCCG CTGGACTCCA TCCGTGAAGC TGACGGCATG ACTCTGCGGG |
| | | TTGCACTTGG TGCCAAACCG CATTTGGGTC GCAGCAAGGG CCAGCAGATC |
| | | GTGATCCCTA CGCCGATCCT CACGCATCTT GATATGCTGC AACTGCGCGA |
| | | GCAGACTGTG GCCCCTTATG CCCGCTTTGA AATGCTCTAT ACGCCGGTCA |
| | | CCGGCAGCGA TGCCGCAAGC CGGGCGGCCA ATGCCAACGC GCTGGAGCAC |
| | | GCCATTGATG AACTGGCGCA GCAGGTGGTT GACTTCGCCC GTGAACAGGG |
| | | TGGCATCGCG GTGGTAACCG ACCGCCATCT CTCCACCACG CGCGCCGCCA |
| | | TCCCGATGCT GCTGGTGGTT TCTGCCATTA ACCAGCGGCT GGTGCAGGAA |
| | | GGTTTGCGTC TGGACGTATC GCTGGTGGTT GAGAGCGGCC AGAGCATCTC |
| | | TTCCCACCAT ATCGCCGCAG CGCTGGGCTT TGGCGCTTCT GCCATCTATC |
| | | CACTGGGCGT GCAGATGCGT GCCGAAGAGA AATATGGCGA GGGTGAAGGC |
| | | GGCAATAAAG CCTTCAAACG CTATGCCAAA GCCGCTGAGA AAGCGCTGAT |
| | | GAAAACCATG GGCAAAGTCG GCCTGTGTAC CGTGGAAAGC TACAGCTGCG |
| | | GCGAGTTCTT TGAGCCCAAC TTCCTTGATA CCGAAGACCG CGTGCTGAAG |
| | | AAGTACTTCC CCAACATCAA AACCCCGGTT GGCGGCGCTG GCTTTGCCTC |
| | | CATCGCGCAG ATGGCGGTTG ACTGGCACCA GAGTGCGCTG CGCGTGCAGG |
| | | GTGAAAATGA CGTTCCGCTA CTCGGCCTGT TCAAAGAGCG GGCAGAAGGC |
| | | GCGGGTCATT CCTACGGCAC CATTGCGGTG CGTACCTTTA TTGATATGAC |
| | | GGAGCAGCCT GTCCGCTTTG CCGATAAGCC GCGCGAGGAG GATAACTTCA |
| | | TCCGCCTGAT GACGCTGGCA AAACTGGATA ATGCGTTTAA TATCAAGGCA |
| | | AAATCCTTTG CTGACAGCAG TTTTGAACGT ATCCCGAACG AGGTTATCGA |
| | | TAACTTTTCC ATTACCGCTG ACTATCGCCA GTTCTCCAGC CTGATGCTGG |
| | | AAGAGCGCAA ACGCCGCCCG GCGGCGCTGC GCGATATTCT TGCTTTCCCG |
| | | GCAGATTTAA CCCACGTCGA CAGCCAGGCG GAATTTACCC GCAAGCTGGG |
| | | CCGTTATTCG CTGGTTAACA ACGGCTTTGC GGTGCGCGGC CTGGAATGCG |
| | | AGGCCGTGGC AGAATGTGCA GGCCAGTTCA GGCTGCGCCT GACCGATGCG |
| | | GTCGCCGGAC TGAAAAGCGA GCAGGAACGC CTGACCACGC TGTGCGGCGT |
| | | GCTGACCAAA CGCTTCGGTG ACGCTATCGA ACGCGCCGAG GTGACGGCGG |
| | | ATGCGCTGCT GCTTACGGCC AGTGGTAAGG CGGCTGAGTA TCTGGCGCGC |
| | | ATCTTCACTA TTGCTCCGGC ATTGCCGCTA AGCGAAGTGC AGCCGGCCCA |
| | | TGAAATTACC CGCACCTTCG CCTCCGGGGC GATGAGCCAC GGCGCGCTGG |
| | | TCGCTCCGGC GCACGAAGCG GTGGCGCACG GCACCAATAT GGTCGGCGGC |
| | | ATGAGCAACT GTGGCGAAGG GGGCGAGCAC TACTCGCGTC ACGGCACCCT |
| | | CCGCGCATCA CGCATTAAGC AGCTGGCATC CGGTCGTTTC GGCGTCTGGG |
| | | CCGCCTATCT GGCCGACCCG ATGCTGGAAG AGCTGGAAAT TAAGATTGGC |
| | | CAGGGGGCGA AGCCCGGCGA AGGCGGCCAG CTGCCGTCGG CCAAAGTAAC |
| | | GGTAGAAATC GCCGCCGCGC GTGGCGGTAC GCCAGGCGTG GAGCTGGTAT |
| | | CCCCTCCTCC GCACCACGAT ACCTACTCAA TCGAGGATCT GGCGCAGCTG |
| | | ATCCACGACT GTAAAGCCGC CCGCGTGCGG GTGATCGTCA AACTGGTTTC |
| | | CTCTGAGGGT ATTGGCACCA TCGCCGTTGG CGTGGCCAAA GCCGGCGCTG |
| | | ACGTGATCAA CGTGGCCGGC AATACCGGCG GCACCGGGGC AGCTTCGGTA |
| | | ACCAGCCTGA AATACACCGG GCGCGTGGCG GAAATCGGCA TCGCTGAAGT |
| | | TCATCAGGCG CTGTGTGCTA ACGGCCTGCG TGAGAAAGTC CTGCTGCGCT |
| | | GCTCCGGTGC GCAGCAAACC GGCAGCGATG TGGTGAAATC CGCGCTGCTG |
| | | GGCGGCGACA GCTTCGAGTT CGGTACCACG GCACTGATGA TGCTGAAGTG |
| | | CGTGATGGCG AAAAACTGCA ATGTGAAATG TCCGGCGGGG CTGACCACTA |
| | | ACGCCGAAGC ATTTGACGGC GACCCGCGCC AGCTGGCGCA GTATTTCATC |
| | | AACGTGGCAC ACGAAGTGCG CGAGATGCTG GCGCGGCTTG GCCTGCGTTC |
| | | ACTGCGGGAA GCCCGTGGCC GCTCCGACCT GCTGCACCTG ATGGATCATC |
| | | CGCTGGAAGT GGGCAAGCTG GATCTGCGCG CCATGCTGAC GGTGGTTCCC |
| | | GAAATGAAAA TCAGCCATCC GGTGTATCTG GAAAAAGATT TTGCGCTGGA |
| | | TAACGGCTGG ATCGATCGGT TACAGGAGCA GCTGGTAATC CAGGGATCGG |
| | | GCGCGATCGT ACTCGGTGAC GGTATCACCC TGAACAACCG CAATAAAAGC |
| | | GTGGGTGGAC AGCTGGCTAT CGATATCGAA CGCCTGCTCA ATCACCAGCT |
| | | GAACGCCGAA CATCTGAGCC GCATTCCGGC TGCGCTACAG GACGATCGCG |
| | | GCCGCCGCTA TCTTGCCCCG GCCACGGTGA CCGTCACCAC CTGCGGTTCC |
| | | GCCGGGCAGT CTTACGGCGT GTTCTGTAAT GACGGCATGC GCCTGCAGCA |
| | | CCAGGGCACC TGTAATGACG GCGTTGGTAA AGGCCAGTGC GGCGGCGAGA |
| | | TCGTGGTGAG ATCGCCTGGC GGTGGTTCAC AGGACGCGGA AGGCAACGTG |
| | | CTGATTGGCA ACTTCGCGCT GTTTGGCGCT ACCGGCGGGC GTCTGTTTGC |
| | | CCAGGGCCAG GCGGGCGACC GCTTTGCCGT GCGTAACTCG GGAGCGACGG |
| | | CGGTGGTCGA AGGCGTTGGT GATTTCTGCT GTGAATATAT GACCAACGGC |
| | | GCGGTGCTGA ATCTTGGCAC CTGTGGAACG GGTTTCGGTA ACGGTATGAG |
| | | CGGCGGCTTC GCCTATCAGT ACGATCCGTA TGGCACGCTG GCGGATTGCG |
| | | CCGCCGCCGA CTCGGTAATG CTCGGTGCCA TCAGCGATGA CAATGAAATG |
| | | GCCCGCGTGC ATCAGCAGGC GGTACTGACC ATGCTGAACT GGCATCTGGA |
| | | AGCGACCGGC TCGCCACGCG CCCGGTGGTT GCTCGACCAC TGGGAAACGG |
| | | AATGCCAGCA TTTTGTTTAT GTGATGCCGC GTTCTCTGCT GCTGTATCAG |
| | | GACGCGACTG AAATCCTTAA GGCAAAAGCG CGTAAGGACC TGCTGGAAGA |
| | | ACTTTCTACC GCTCTGGCCA GTCACCAGGT GGCGAAATTC AAAGCCGCAT |
| | | GGCGCGCCGG CAAACCGATC GCCGACGGCT CGGTTCCGGT CTACGGCGCC |
| | | ATGGACACCC CGGAAATGCT GGTGCTGCTG AATAATTATA CCGTCCTGAG |
| | | CTGTGCCCAG CAGCTGGCGC TGTCCCGTCT GCCGAAAGGC ACGGCGGTGC |
| | | AGGACCCGGC GGTGGAGAAA GCGGTGCGTA ACCTGCTGAT GACAGAAGAT |
| | | TTTGCTCTGA TTAGTAAACT GCAGCGCCAC GGTCGTGCGG CGATTGAAAG |
| | | CTACAGCGAT GAGCAGCTGG CCTGCCTGAT CGGTGCCAAA CGCATGGCGG |
| | | ACTATAAAGC GGCGCTGACG CAGCGCAATA TCCGTTCGAT GGACAGTCTG |
| | | GCCACCTATG GCTGGATCCA GTATCAGGAT GCGTGTAACA GGGAAGTGCT |
| | | GGGACGACTG CCTGACTTTG AAGAGCTGTT TGCCCGCGCC GCACTGCCTG |
| | | AGTTAGCGGC GGCGGTCGGC AAACTGGCCG GATAA |
| 25 | Erwinia | ATCGATCCTT TAGATTGTTC CGTCGAGTAT ACGGCAGCAT TGTCAGGCAC |
| | amylovora | AGTGGCATGC GCGGAAAAGC AAAAACCGGC CATCGCAACC GATGCAGCCG |
| | foxR | TAATAATAAG TTTCATATTA ACTCCCTTAT TTGGGTTTGA ATTTTGCTTA |
| | | ATCTACCTGG TCAAGGCAAA GTGGTGAAAC AGTCCTGTCC CGGCAAGCTA |
| | | AACCAGCGAT AAGTATAGCC AGTGTTTACC GTTTCATCGG GCGCTACAGC |
| | | AGCCCCGCCG GATTGGTGTC TGTGCTGTCC GGCTGGAACA CAATGCGAAT |
| | | CCTGGCCTTG ATGGCAAAAC CTGCTGTGCC AGCCGGCGTA TATCAGCAGC |
| | | AGATGGGGCA GGTGCTGCGG GCCTGATTAA TCGAGGCCGT TAAAGACGCT |
| | | TGTCTCTATC TCGCTGTATT AGAAATAAAT ATGGTCACGT CTTGAAATAC |
| | | GACATAAAAA AAGATAATAA TTCTCATTTA CATAGTAAAA TCGGTTACAT |
| | | TCAGGTTATG CGCGCCTGTC TGCTCTGGAT CCATCCGCGA AATAAGAGTA |
| | | ATAAGTCTAA TCTTTGTAAG GGAATAAACA TGAGAATGCC TTTTAGCCTC |
| | | AAGCGTTCTG TGCTGCTCTG TGGCATAGCA CTTTTCACGC CGGGTATGAC |
| | | CTGGGCCGCA GATACGCTGG TGGTTACCGC TAAACCGGAA GAAACCGCCA |
| | | GCACGCCGAC GGAAGGCTAC ACCGCGAAGA TCAGCAGCGG GGCGACCAAG |
| | | ACCGATCGCC CATTGATCAC CACCGGCCAG TCCGTTTCCG TGGTGACTCG |
| | | TCAGCAGATG GAAGATCAGG GGGCGATGGA CGTTAATCAG GCGCTGAATT |
| | | ACACCGCCGG GGCCTTTACT AACTTTGCTG GCGCGGCATC ACGTTATGAT |
| | | ACGGTATCGT TGCGTGGATT CCACGGCGGC GATGTCGACA ACATTTTTGT |
| | | TGATGGCCTG CGCCTGATGA GCGATCCGGG TAGCTTCAAT GCGTTGCAGA |
| | | TTGATCCGTG GTTTCTCGAC AGGATCGACG TGATCAAAGG CCCTTCTTCC |
| | | GCTCTGTATG GTCAGACCGT GCCGGGTGGG CTGGTGATGG AAAGCTCTAA |
| | | ACGCCCGCAG TTTGCCCCTG AAGGCCATTT CCGCCTGTCG ACCGGCTCCA |
| | | ACAGTACCAA CAGCGCGGCG TTTGATTACA CCGATGCAAT CAACGAGCAA |
| | | TGGGCGTTTC GTCTGACCGG CATTACCCGT AGCAGTCATA CCCAGTACGA |
| | | TCATACCCGC GAAGAAAAAT ACGCCATTTC CCCGTCGCTG CTGTGGCAGC |
| | | CGGATGAGGA TACGTCCCTG CTGCTTCGTG CCTATCTGCA AAAAGATCCT |
| | | TCCGGCGGTT ACCACAGTGC GGTTCCGGGT GAGGGCAGCA TTACCGCACA |
| | | CAACGGCAGA AAACTGAGCA CCGGTTTTTA TGACGGCGAC AGTTCATTGG |
| | | ATCAGTTTAA ACGCCGGGAG CAGATCTACA GCGCTCAATT CTCGCACCGC |
| | | TTTAACGACA CCTGGGCGTT TCGTTCCAAC GCCAGCTATT CACATTCAAA |
| | | TGTCGATCTC GACCAGGTTT ATCAGATTGG CTGGGACACT AATAACGCCG |
| | | ATCTGTTGAA TCGTTACTAT TCAGGATCGC GATCATCGCT AAATGCCTTT |
| | | GCCATCGATA ATCAGCTGGA AGCCGACTTC GCTACCGCGG AAGTTGCTCA |
| | | CAGAGTAGTA CTGGGAGCTG AATATCACCA GTACAAGAAT GACCTGAGTG |
| | | ATGCCAGAGG TTCGGCCAGC CAGCTCAATG CCCTTACCGG CCAGGTGGTG |
| | | GGCAGCAATG ACAATTTTAC CTTCTCTAAA TCGCAGCGCC GTTACTATCA |
| | | GGCCGGGGTT TATCTGCAGG ATGAAATGAC GTGGGATAAA TGGCATCTGG |
| | | ATCTCTCCGG CCGCTACGAT CGCCTCGTTG CCCGGACCGA CAACATTGAC |
| | | CAGGAGACTA AATTCCGTCG CCAGGACGAC CACGTCGGCG GGCGCGCATC |
| | | CCTGCTGTAT GCGTTTGATA ACGGTATTTC CCCTTATGCC AGCTACAGCC |
| | | AGGCGATTAC GCCGCAGTCT CTTCCTGGCA AGGACGGCAA TGTGCTGAAA |
| | | CCGACGACTT CTGAGCAGTA TGAAGCCGGG GTGAAATACC AGCCGGTGGG |
| | | AACGTCGGAT CTCTATTCCA TCGCCGTCTA CGATCTGACG CAAAAAGATG |
| | | TCGGCAACCG CGTTATTGTA GGCTCTTATT ATGTCCCAGC CGGGAAAGTT |
| | | CATTCACAGG GCCTGGAACT GGAAGCCCAC AACCAGCTGA CGCCGCGACT |
| | | CAGTACCCTT GCTACCTACA CGTTGAATCA CGTACGTTAT AAGGATGCCA |
| | | TTGATGGCAA TGACGGCCAC ACCCCTTACG TTACGCCAAA TTCGATGGCT |
| | | TCAGCCTGGG CCAAATACCA GTTTGACTAC GGGATTAGCG TGGGGGCCGG |
| | | GGTGCGCTAC ATCGGCAAGC AGTGGGCGGA TAACGCAAAC ACTACCCGTC |
| | | TGCCATCGGC CACGCTATTT GACGCGTCCG TTCGTGCCGA TCTCGGCGCC |
| | | TGGAACAGCA GCCTGAAAGG GGCCTTTGTG CAGGTAAATG CCAATAACCT |
| | | GACCGATCGC GACTACGTTT CTGCCTGTTA CGGTACCGGG TATTGTTATT |
| | | GGGGCGCTGA ACGTACGGTG ATGGCTACCG TCGGTTACGA TTTCTAACCC |
| | | GCGATTCGGT TTACCTGAAT AAAAAGCACG GCCGTTTAAG CGGCCGTGCT |
| | | TTTTTTTACC CAGAAGAAGG AGGACGTT |
| 26 | Erwinia | ATGTGTGGAA TTGTTGGTGC TGTAGCGCAG CGTGATATTG CAGAAATCCT |
| | amylovora | GCTCGAAGGG CTGCGCCGTC TGGAATACCG TGGCTATGAC TCTGCCGGTC |
| | glmS | TGGCCGTGGT TGATGCTAAC GGGCAGGCTA CCCGCCTGCG TCGCGTTGGC |
| | | AAGGTGAGCA ACCTGACTGC CGCGGCGGAA AACACCGCGC TGGCGGGTGG |
| | | AACCGGTATC GCGCACACGC GCTGGGCCAC GCACGGCGAA CCGTCGGAAA |
| | | GTAATGCGCA CCCGCACATT TCCGGCGAAA TCATTATTGT TCACAACGGC |
| | | ATTATTGAAA ACCATGAGCC ACTGCGCGAG CTGATGACAG GCCGCGGTTA |
| | | CACCTTTACC TCAGAAACTG ATACCGAGGT GGTGGCGCAC CTGGTGCACT |
| | | GGGAACAGCA AAACGGCGGC TCACTGCATG AAGTGGTGCA GCGTGTCATC |
| | | CCGCAGCTGC GCGGTGCTTA CGGTATGGTT ATCCTTGACA GTCGCGATCC |
| | | CGCCACGCTG ATAGCTGCGC GCTCCGGCAG CCCGCTGGTA ATAGGCCGTG |
| | | GCGTGGGTGA AAACTTTATT GCCTCAGATC AGGTGGCGCT GCTGCCGGTT |
| | | ACGCGCCGCT TTATCTATCT GGAAGAGGGT GATATCGCGC AAATTACCCG |
| | | CCGTAACGTC ATTATCGTTG ACCGCAGCGG CGAGGTTGTA CAGCGCCGCG |
| | | AAATCGAATC CAATACGCAG TATGATGCCG GCGATAAAGG TGCCTACCGC |
| | | CACTACATGC AGAAAGAGAT TTACGAGCAG CCGGTGGCGA TTAAAAACAC |
| | | CCTTAGCGGT CGTTTCAGCC ACGGAGAAGT CGACTTTTCC GAGCTGGGCG |
| | | CCAACGCTGA CTCGCTGCTG GCCAACGTTG AACATATCCA GATTGTCGCC |
| | | TGTGGCACCT CATACAACTC TGGTATGGTT TCGCGCTACT GGTTTGAATC |
| | | TCTGGCCAAC GTACCTTGTG ACGTGGAGAT CGCTTCTGAA TTCCGCTACC |
| | | GCAAATCTGC GGTGCGCAAA AACAGCCTGC TGATCACCCT TTCTCAATCC |
| | | GGTGAAACCG CCGATACGCT GGCAGCGCTG CGCCTGTCGA AAGAGCTGGG |
| | | TTATTTGGGA TCGCTGGCGA TCTGCAACGT GGCGGGCTCC TCCCTGGTGC |
| | | GCGAATCTGA CCTGGCGTTG ATGACCAAAG CCGGTACGGA AATCGGCGTG |
| | | GCATCAACCA AAGCCTTTAC CACCCAGCTA ACCGTCCTGC TGATGCTGGT |
| | | GGCAAAAATT GGTCGTCTTA AAGGCGTTAA TCCACAGGTG GAACACGATA |
| | | TTGTTCACTC CCTGCAGGCG TTGCCGAGCC GTATTGAGCA GATGCTGTCA |
| | | CAGGATGAGC TGATTGCTTC CCTGGCAGAG GATTTCTCAG ACAAACATCA |
| | | CGCGCTGTTC CTTGGCCGTG GCGATCAGTA CCCGATCGCC ATGGAAGGGG |
| | | CGCTGAAGCT AAAAGAGATC TCCTATATTC ACGCTGAAGC CTACGCCGCC |
| | | GGAGAGCTGA AACACGGCCC GCTGGCGCTG ATCGATGCTG ATATGCCGGT |
| | | GATTGTTGTC GCGCCGAACA ATGAACTGCT GGAAAAGCTG AAGTCGAACA |
| | | TTGAAGAAGT GCGCGCCCGT GGCGGCCTGC TGTATGTCTT CGCCGATGCG |
| | | GATGCCGGTT TTGCCAACAG CGAAGGGATG CGTATCATTC CGCTGCCGCA |
| | | TGTCGAAGAG GTGATCGCGC CAATCTTCTA CACCGTGCCG TTGCAGCTGC |
| | | TCAGCTACCA TGTGGCGCTG ATTAAAGGCA CCGATGTAGA CCAGCCGCGC |
| | | AACCTGGCGA AATCGGTGAC GGTGGAATAA |
| 27 | Erwinia | ATGTCTATCG TTGAAAAAAA ACCCACCCGC CGCGAACAGC GCGCCCAGGC |
| | amylovora | GCAGCAGTTT ATTGAGTCGT TGCAGGGCAG TGCCTTTCCC AACTCAAAGC |
| | thiC | GCATCTGGTT GACCGGATCG CAGCCCGATA TTCGCGTGCC GATGCGTGAG |
| | | ATCCAGCTTA GCCCCACCGT AACAGGCGGC AGCAAAGATA ACCCAACGCT |
| | | TGTCGGCAAT GAACCGGTGC CGGTCTATGA TACTGCCGGT CCCTATGGCG |
| | | ACCCGGAGGC GAGCATTGAT GTTTATGCCG GGCTGGCCAA ACTGCGTGCT |
| | | GACTGGATCG TTAAACGCGA TGACACAGAA CAGATCGCCG AACTGAGTTC |
| | | CGCCTATACC CAGCAACGCC TGGCCGATGA AGGACTGGAT CATCTGCGTT |
| | | TCGACAATTT GCCCTGTCCT CGCCGCGCCC GTGCCGGCCG CTGTGTGACC |
| | | CAGATGCACT ACGCGCGTCT TGGGGTGATC ACCCCGGAAA TGGAATTTAT |
| | | TGCCATCCGT GAAAATATGG GGCGCGAGCG TATTCGCGCT GAGGTGCTGT |
| | | TACAACAGCA TCCGGGTAAC AGCTTTGGTG CCAGCCTGCC GGAAAATATT |
| | | ACCCCTGAAT TTGTCCGTCA GGAAGTTGCC GCCGGGCGCG CCATTATTCC |
| | | CGCCAATATT AACCACCCGG AATCCGAACC GATGATTATT GGCCGTCATT |
| | | TCCTGGTGAA AATCAACGCC AATATCGGCA ACTCGGCGGT CAGCTCCTCT |
| | | ATCGAAGAAG AGGTGGAAAA GCTGGTGTGG TCAGCGCGCT GGGGCGCAGA |
| | | TACGGTGATG GACCTGTCCA CCGGGCGTTA TATCCATGAA ACCCGCGAGT |
| | | GGATCCTGCG CAACAGCCCG GTTCCCATTG GTACTGTGCC GATCTATCAG |
| | | GCGCTGGAAA AAGTGAACGG CGTGGCGGAA GACCTCAACT GGGAAATCTT |
| | | CCGCGATACC CTGCTGGAAC AGGCTGAACA GGGCGTTGAC TACTTCACCA |
| | | TCCATGCCGG CGTACTGCTG CGCTATGTGC CGATGACCGC CAAACGTCTG |
| | | ACCGGCATTG TTTCGCGCGG CGGCTCGATT ATGGCGAAAT GGTGCCTGTC |
| | | ACATCATCAG GAGAGTTTTC TCTATCTGCA CTTTCGCGAA ATCTGCGAAA |
| | | TCTGCGCCGC TTATGACGTT TCGCTGTCGC TCGGCGATGG CCTGCGCCCC |
| | | GGCTCGATTC AGGACGCCAA TGATGAGGCC CAGTTCGCCG AGCTGCGCAC |
| | | GCTGGGTGAA CTGACTAAAA TCGCCTGGGA ATACGACGTG CAGGTGATGA |
| | | TTGAAGGGCC TGGCCATGTG CCAATGCAGA TGATCCGCAC TAATATGACG |
| | | CAACAGCTGG AACACTGCCA CGAAGCGCCA TTCTACACGC TTGGCCCGCT |
| | | GACCACCGAT ATCGCTCCGG GATACGACCA TTTCACCTCC GGGATTGGCG |
| | | CCGCAATGAT TGGCTGGTTT GGCTGCGCCA TGCTGTGCTA CGTCACACCC |
| | | AAAGAGCATC TTGGCTTGCC GAACAAGGAG GATGTCAAAC AGGGACTGAT |
| | | TACCTATAAA ATTGCGGCCC ATGCCGCCGA CCTGGCCAAA GGCCATCCCG |
| | | GCGCACAGAT CCGCGACAAC GCCATGTCAA AAGCACGCTT CGAATTCCGT |
| | | TGGGAAGATC AGTTTAATCT CGCCCTCGAT CCGCATACCG CCCGCGCCTA |
| | | CCACGATGAA ACTCTGCCGC AGGAATCGGG CAAGGTGGCG CACTTTTGTT |
| | | CGATGTGCGG ACCGAAGTTC TGTTCCATGA AAATCACTCA GGAAGTGCGG |
| | | GAATACGCCG CACGACAGGA AGCGGAGGCA AAGCCTGTCG AGATGGGTAT |
| | | GGCACAGATG TCACGGGAAT TCCGCAGCCG GGGAGGCGAG CTTTATCACC |
| | | GCGCCGATAG CCCGACGCCG GAGGAAAAAG TATGA |
| 28 | Erwinia | ATGAGCGATG AAAAATACCC TGGGCCGCTG GTGGTTGAAG GCAAGCTGGT |
| | amylovora | CGACGCCGAA CGGCTGAAGA AACAGAGCAA TTATCTGCGC GGTGGCATTG |
| | cysI | CCGAAGACCT GCATGACGGG CTGACCGGCG GCTTTAACGG CGATAACTTT |
| | | CTGCTGATCC GCTTTCACGG CATGTACCAG CAGGATGACC GCGATATTCG |
| | | CGCCGAACGC GCCGAACAAA AGCTGGATGC GCGTCATGCG ATGATGCTGC |
| | | GCTGCCGTTT GCCGGGCGGC ATTATGACGC CAAAGCAGTG GCTGGCCATC |
| | | GATAAGTTCG CCAGCGACAA TACCCTGTAC GGCAGTATTC GTTTGACTAA |
| | | CCGTCAAACG TTCCAGTTCC ACGGCATCCT GAAAGGCAAC GTCAAACCGG |
| | | CGCATCAGAT GCTGCACGAA GTCGGGCTGG ACGCGCTGGC CACCGCCAAC |
| | | GATGTCAACC GTAACGTGCT GTGCAGCTCG AACCCGATTG AGTCCGAACT |
| | | GCATCAGGAA GCCTACGAAT GGGCGAAAAA GCTCTCCGAA CATCTGCTGC |
| | | CGCGAACCCG TGCCTATGCG GAGATCTGGC TTGACCAGGA AAAAGTCGCC |
| | | ACCACCGATG AAGAGCCGAT CCTCGGTGAA ACCTATCTGC CGCGTAAGTT |
| | | CAAAACCACC GTGGTGGTTC CGCCGCACAA TGACGTAGAC CTGCACGCTA |
| | | ACGACATGAA CTTTATCGCC ATCGCTGAAC ACGGCAGGCT GGTCGGTTTC |
| | | AACCTGCTGG TCGGCGGCGG TCTTTCTATT GAACACGGAA ATAAAGCCAC |
| | | CTATGCCCGT ACCGCCAGCG AGTTCGGCTA TCTGCCGCTG AGTAAGATAC |
| | | TCGACGTGGC CGAAGCAGTG GTAACCACCC AGCGTGACTG GGGCAACCGT |
| | | ACTGACCGTA AAAATGCCAA AACCAAGTAC ACCCTTGAGC GCGTGGGGCC |
| | | GGATGTGTTT AAGGCAGAAG TCGAGCGCCG TGCCGGTATC ACCTTTGAGC |
| | | CGATCCGACC GTATGAATTT ACCACCCGTG GCGATCGTTT CGGCTGGGTT |
| | | AAGGGCATTG ACAACCAGTG GCATCTCACG CTGTTTATCG AAAATGGCCG |
| | | TCTGCTGGAT TATCCGGGCC GCCCGCTGAA AACCGGCCTG GCAGAAATTG |
| | | CCAAAATCCA CCACGGTGAC TTCCGCCTGA CCGCGAATCA AAACCTGATC |
| | | GTTGCCAGGG TGCCGGAGGC AGAAAAAGAC AATATTCAGC GGCTGGCGGT |
| | | CGCGCACGGT CTGATGGAAA ACGTCAGTCA TCAGCGTGAA AACTCGATGG |
| | | CCTGTGTGGC ATTCCCGACC TGTCCGCTGG CGATGGCGGA AGCAGAGCGT |
| | | TTTCTGCCGC AGTTCGTCAC CCGCGTGGAG GAGATCCTCA CCGCACACGG |
| | | CGTGGGCGAT GAGCATATTG TTCTGCGCGT CACCGGCTGC CCTAACGGCT |
| | | GTGGTCGTGC GCTGCTGGCG GAAATCGGCC TGGTGGGGAA AGCGCCGGGG |
| | | CGCTATAACC TGCACCTTGG CGGTAACCGC ATTGGTACGC GCATCCCGCG |
| | | CATGTATCGC GAAAATATCA ACGAAGAGCA AATTCTGGCA ACCCTTGATG |
| | | AACTGATCGG CCGCTGGTCG CAGGAGCGCG AAGCCGATGA AGGCTTCGGT |
| | | GATTTCACCA TCCGTGCCGG AGTGGTGCAA CCGGTACGCG ATCCGGCCCG |
| | | CGATTTCTGG CAATGA |

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method of reducing the occurrence of or preventing fire blight disease in a pome fruit tree comprising:
applying a non-transgenic Erwinia amylovora strain comprising one or more auxotrophic mutations to a pome fruit tree growing in a field under conditions effective to reduce the occurrence of or prevent fire blight disease.

2. The method of claim 1, wherein the one or more auxotrophic mutations are selected from the group consisting of: a leucine auxotrophic mutation; an arginine auxotrophic mutation, a proline auxotrophic mutation; a threonine auxotrophic mutation; a tryptophan auxotrophic mutation; a guanine auxotrophic mutation; a purine auxotrophic mutation; a pyrimidine auxotrophic mutation; a sugar utilization gapA auxotrophic mutation; a sugar utilization ptsI auxotrophic mutation; a nitrogen utilization glnA auxotrophic mutation; a nitrogen utilization gltB auxotrophic mutation; a high affinity siderophore-based iron uptake auxotrophic mutation; a glucosamine auxotrophic mutation; a thiamine auxotrophic mutation; an inorganic sulfur uptake auxotrophic mutation; and combinations thereof.

3. The method of claim 1 and claim 2, wherein the one or more auxotrophic mutations is a leucine auxotrophic mutation.

4. The method of claim 3, wherein the leucine auxotrophic mutation is the Leucine-1 auxotroph.

5. The method of claim 1 and claim 2, wherein the one or more auxotrophic mutations is an arginine auxotrophic mutation.

6. The method of claim 5, wherein the arginine auxotrophic mutation is the Arginine-1 auxotroph.

7. The method of any one of claims 1-6, wherein the one or more auxotrophic mutations is a UV-induced mutant.

8. The method of any one of claims 1-7, wherein said applying further comprises:
applying the non-transgenic Erwinia amylovora strain comprising one or more auxotrophic mutations to a pome tree at flowering.

9. The method of claim 8, wherein the pome fruit tree has at least 50% of flowers in bloom.

10. The method of any one of claims 1-9, wherein said applying comprises an effective amount of the non-transgenic Erwinia amylovora strain comprising one or more auxotrophic mutations to reduce the occurrence of or prevent fire blight disease caused by Erwinia amylovora.

11. The method of claim 10, wherein the effective amount is about 10⁶-10⁹ CFU/ml.

12. The method of any one of claims 1-11, wherein said applying comprises contacting the pome fruit tree with a composition comprising the non-transgenic Erwinia amylovora strain comprising one or more auxotrophic mutations and an agriculturally acceptable carrier.

13. The method of claim 12, wherein the agriculturally acceptable carrier is water.

14. The method of claim 12 or claim 13, wherein the composition further comprises a milk product.

15. The method of any one of claims 12-14, wherein the composition further comprises a surfactant.

16. The method of any one of claims 12-15, wherein the composition further comprises one or more compounds for which the Erwinia amylovora strain comprising one or more auxotrophic mutations is auxotrophic.

17. The method of claim 16, wherein the one or more compounds is leucine and/or arginine.

18. The method of claim 17, wherein the leucine is provided at 1-100 mM.

19. The method of claim 17, wherein the arginine is provided at 1-100 mM.

20. The method of any one of claims 12-19, wherein the non-transgenic Erwinia amylovora strain is present in the composition as a wet paste or in a lyophilized form.

21. The method of any one of claims 1-20, wherein said applying is carried out by spraying.

22. The method of any one of claims 1-21, wherein said applying reduces fire blight disease by at least 10% compared to an untreated pome fruit tree.

23. The method of any one of claims 1-22, wherein said applying reduces occurrence of russetting on pome fruits of the treated pome fruit tree compared to pome fruits of a pome fruit tree treated for fire blight disease with an organic compatible treatment other than the non-transgenic Erwinia amylovora strain comprising one or more auxotrophic mutations.

24. The method of any one of claims 1-23, where the pome fruit tree is located in an environment susceptible to fire blight disease mediated by Erwinia amylovora.

25. The method of any one of claims 1-24, wherein said applying reduces occurrence of or prevents fire blight disease of blossoms of a pome fruit tree compared to blossoms of a pome fruit tree not treated with non-transgenic Erwinia amylovora strain comprising one or more auxotrophic mutations.

26. The method of any one of claims 1-25, wherein the pome fruit tree is selected from the group consisting of apple tree, pear tree, pomegranate tree, Asian pear tree, quince tree, loquat tree, and crabapple tree.

27. An agricultural composition comprising:
a non-transgenic Erwinia amylovora strain comprising one or more auxotrophic mutations and
an agriculturally acceptable carrier.

28. The agricultural composition of claim 27, wherein the one or more auxotrophic mutations are selected from the group consisting of: a leucine auxotrophic mutation; an arginine auxotrophic mutation, a proline auxotrophic mutation; a threonine auxotrophic mutation; a tryptophan auxotrophic mutation; a guanine auxotrophic mutation; a purine auxotrophic mutation; a pyrimidine auxotrophic mutation; a sugar utilization gapA auxotrophic mutation; a sugar utilization ptsI auxotrophic mutation; a nitrogen utilization glnA auxotrophic mutation; a nitrogen utilization gltB auxotrophic mutation; a high affinity siderophore-based iron uptake auxotrophic mutation; a glucosamine auxotrophic mutation; a thiamine auxotrophic mutation; an inorganic sulfur uptake auxotrophic mutation; and combinations thereof.

29. The agricultural composition of claim 27 or claim 28, wherein the one or more auxotrophic mutations is a leucine auxotrophic mutation.

30. The agricultural composition of claim 29, wherein the leucine auxotrophic mutation is the Leucine-1 auxotroph.

31. The agricultural composition of claim 27 or claim 28, wherein the one or more auxotrophic mutation is an arginine auxotrophic mutation.

32. The agricultural composition of claim 31, wherein the arginine auxotrophic mutation is the Arginine-1 auxotroph.

33. The agricultural composition any one of claims 27-32, wherein the one or more auxotrophic mutations is a UV-induced mutation.

34. The agricultural composition of any one of claims 27-33, wherein the agriculturally acceptable carrier is water.

35. The agricultural composition of any one of claims 27-34, wherein the agriculturally acceptable carrier further comprises a milk product.

36. The agricultural composition of any one of claims 27-35, wherein the composition further comprises a surfactant.

37. The agricultural composition of any one of claims 27-36, wherein the composition further comprises one or more compounds for which the Erwinia amylovora strain comprising one or more auxotrophic mutations is auxotrophic.

38. The composition of claim 37, wherein the compound is leucine and/or arginine.

39. The composition of claim 37 or claim 38, wherein the compound is 1-100 mM leucine.

40. The method of claim 37 or claim 38, wherein the compound is 1-100 mM arginine.

41. The agricultural composition of any one of claims 27-40, wherein the non-transgenic Erwinia amylovora strain is present in the composition as a wet paste or in a lyophilized form.

42. The agricultural composition of any one of claims 27-41 wherein the non-transgenic Erwinia amylovora strain comprising one or more auxotrophic mutations is present in the composition in an amount effective to reduce or prevent fire blight disease on pome fruit trees caused by Erwinia amylovora.

43. The agricultural composition of any one of claims 27-42, wherein the non-transgenic Erwinia amylovora strain comprising one or more auxotrophic mutations is present in the composition in an amount of about 10⁶-10⁹ CFU/ml.

44. The agricultural composition of any one of claims 27-43, wherein the non-transgenic Erwinia amylovora strain comprising one or more auxotrophic mutations is present in the composition in an amount of about 10⁹ CFU/ml.
